(19) United States
(12) Patent Application Publication
(10) Pub. No.: US 2026/0152808 A1
(43) Pub. Date: Jun. 4, 2026

(54) A PREDICTIVE SCORE OF CANCER IMMUNOTHERAPY OUTCOME BASED ON ECOLOGICAL ANALYSIS OF GUT MICROBIOTA

(71) Applicants: INSTITUT GUSTAVE ROUSSY, VILLEJUIF (FR); INSTITUT NATIONAL DE LA SANTE ET DE LA RECHERCHE MEDICALE, PARIS (FR); UNIVERSITE PARIS-SACLAY, GIF-SUR- YVETTE (FR)

(72) Inventors: Lisa DEROSA, PARIS (FR); Valerio IEBBA, TRIESTE (IT); Laurence ZITVOGEL, PARIS (FR)

(21) Appl. No.: 19/125,379

(22) PCT Filed: Nov. 2, 2023
(86) PCT No.: PCT/EP2023/080606

§ 371 Date: Apr. 29, 2025

(30) Foreign Application Priority Data
Nov. 4, 2022 (EP) 22306675.4

(51) Int. Cl. C12Q 1/689; A61K 35/741; A61K 39/00; A61K 40/11; A61K 40/31; A61K 45/06; A61P 35/00; C07K 16/28; C12Q 1/04

(52) U.S. Cl. CPC C12Q 1/689; A61K 35/741; A61K 40/11; A61K 40/31; A61K 45/06; A61P 35/00; C07K 16/2818; C07K 16/2827; C12Q 1/04; A61K 2039/505; C12Q 2600/106

(57) ABSTRACT

The present invention relates to a score (TOPOSCORE) for describing eubiosis or dysbiosis in an individual, that can be used, inter alia, for determining if a patient is likely to respond to an immune-oncology treatment, more precisely, a treatment comprising administration of an immune checkpoint inhibitor (ICI). The TOPOSCORE represents a robust biomarker predicting immunosensitivity and immunoresistance to ICI on an individual basis.

### FIELD OF THE INVENTION

The present invention relates to the field of gut microbiota and identification of dysbiosis. Dysbiosis is known to be a cause of treatment failure in anticancer therapy. The present invention relates to a score for describing eubiosis or dysbiosis in an individual, that can be used, inter alia, for determining if a patient is likely to respond to a cancer treatment, more precisely, a treatment comprising administration of an immune checkpoint inhibitor.

### BACKGROUND OF THE INVENTION

Microbial symbionts inhabiting our mucosae perform complex functions impacting biogeochemical cycles and human health (Cho et Blaser 2012). The biological properties of microbial communities are determined by their taxonomic composition. In fact, multiple chronic inflammatory disorders including cancer have been causatively linked to shifts in the gut microbiome (Gilbert et al. 2018; Gacesa et al. 2022). Hence, tumorigenesis can induce a stress ileopathy that promotes a protracted intestinal dysbiosis characterized by the relative over-representation of the immunosuppressive Enterocloster genus that induces resistance to PD-1 blockade (Yonekura et al. 2021). Fecal microbial transplantation may circumvent primary resistance to immunotherapy in melanoma, when inducing distinct ecological changes accompanied by anti-inflammatory, immunological and metabolic reprogramming of the original microflora (Davar et al. 2021) and tumor microenvironment of the recipient. Indeed, clinical benefit to immune checkpoint inhibitors (ICI) or chimeric antigen receptor (CAR)-T cell therapy has been linked to the presence or absence of distinct intestinal commensals across various malignancies (Routy et al. 2018; Gopalakrishnan et al. 2018; Zitvogel et al. 2018; Smith et al. 2018). Antibiotics (except vancomycin) (Yonekura et al. 2021; Vétizou et al. 2015), proton pump inhibitors and prebiotics alter the taxonomic composition of the intestinal microbiota, resulting in resistance to immunotherapy (Derosa et al. 2018; 2021; Spencer et al. 2021). Beneficial gut ecosystems, comprising, among others, several Lachnospiraceae and Ruminococcaceae family members, and species from Faecalibacterium, Akkermansia and Bifidobacterium genera harbor pattern recognition receptor ligands, produce metabolites (such as short chain fatty acids, L arginine, inosine, or tryptophane), and express cancer antigen mimetics that can elicit type 1 interferon (IFN) or interleukin-12 (IL12)-mediated TH1 or follicular T helper cell responses during immunotherapy (Mager et al. 2020; Roberti et al. 2020; Overacre-Delgoffe et al. 2021). Although there is compelling evidence for beneficial and/or harmful metagenomic species (MGS) associated with clinical outcome in at least 18 ancillary studies (Park et al. 2022), little consensus exists on which microbiome characteristics are commonly associated with responses and whether a user-friendly tool could be developed to diagnose clinically significant intestinal dysbiosis in patients with cancer (Newsome et al. 2022; Lee et al. 2022; McCulloch et al. 2022).

Several confounding factors may have contributed to this lack of consensus, such as technical considerations (fecal sample collection methodologies and DNA extraction protocols), geographical differences in patient populations (different diets and medication-use across different countries), statistical reasons (such as inter-patient variability, small sample size) and the significance of microbial signals that are functionally related but driven by different species (McCulloch et al. 2022). While the interest of oncologists and patients in defining intestinal commensal communities has dramatically increased over the last 5 years, our understanding of microbial interactions within communities is lagging behind our ability to describe the metagenome. Moreover, it remains difficult to predict which group of microbes would form a stable community or how a given community would respond to intrinsic (pathological) or external (therapeutic) perturbations. While resource competition, metabolic cross-feeding and niche availability are among the main drivers of microbial community assembly (Friedman et al. 2017; Clark et al. 2021; Sanchez-Gorostiaga et al. 2019) the effect of host genetics on the gut microbiome is still being elucidated.

Hence, although accumulating evidence point to the clinical impact of the intestinal microbiota on immunotherapeutic outcomes across various cancers, and although specific gut microbial species have been associated with beneficial responses in meta-analyses, no consensus exists on a gut fingerprint predicting immunoresistance. Obviously, a marker enabling the identification, on an individual basis, of an “immunoresistance-related dysbiosis”, would be of tremendous interest to avoid treating a patient with an immune-oncology (I-O) therapy when said patient is likely not to respond to this therapy. Indeed, these therapies are costly and can cause severe side-effects.

The present invention aims at providing a tool to assess, for an individual, his or her chances to benefit from or to resist to an I-O therapy due to intestinal dysbiosis.

### SUMMARY OF THE INVENTION

Based on shotgun metagenomics sequencing of fecal materials at baseline, the inventors constructed a co-abundance network depicting relative abundance interrelationships within a discovery cohort of 245 patients with advanced non-small cell lung cancer (NSCLC). This network identified several microbial consortia, or communities, named “Species Interacting Groups (SIG)”, leading to the identification of two main SIG driving the clinical response to PD-1 blockade in advanced NSCLC. These two SIG were dramatically enriched with either 40 harmful (SIG1) or 34 beneficial (SIG2) bacteria.

The 40 harmful bacterial species of SIG1 are Dialister invisus, Enterococcus faecalis, Haemophilus parainfluenzae, Veillonella atypica, Eggerthella lenta, Erysipelatoclostridium ramosum, Enterocloster bolteae Alloscardovia omnicolens, Bifidobacterium dentium, Campylobacter concisus, Clostridium perfringens, Enterococcus durans, Enterococcus faecium, Klebsiella pneumoniae, Lacticaseibacillus paracasei, Lacticaseibacillus rhamnosus, Lactobacillus delbrueckii, Lactobacillus gasseri, Lactobacillus vaginalis, Lactococcus lactis, Lactococcus laudensis, Ligilactobacillus salivarius, Limosilactobacillus fermentum, Limosilactobacillus oris, Megasphaera micronuciformis, Mogibacterium diversum, Scardovia wiggsiae, Streptococcus anginosus, Streptococcus gordonii, Streptococcus infantis, Streptococcus mutans, Streptococcus parasanguinis, Streptococcus salivarius, Veillonella dispar, Veillonella parvula, Veillonella rogosae, Enterocloster aldensis, Enterocloster asparagiformis, Faecalimonas umbilicata and Gordonibacter urolithinfaciens.

The 34 beneficial bacterial species of SIG2 are Dorea formicigenerans, Dorea longicatena, Eubacterium ventriosum, Eubacterium rectale, Holdemania filiformis, Parasutterella excrementihominis, Anaerostipes hadrus, Blautia obeum, Blautia wexlerae, Faecalibacterium prausnitzii, Roseburia hominis, Roseburia intestinalis, Roseburia inulinivorans, Ruminococcus bicirculans, Ruminococcus lactaris, Candidatus Cibiobacter qucibialis, Clostridiales bacterium KLE1615, Faecalibacillus intestinalis, Lachnospira eligens, Lacrimispora celerecrescens, Adlercreutzia equolifaciens, Agathobaculum butyriciproducens, Anaerobutyricum hallii, Blautia faecis, Blautia massiliensis, Clostridia unclassified SGB4447, Clostridiaceae bacterium, Clostridium sp AF34 10BH, Clostridium sp AF36 4, Eubacteriaceae bacterium, Fusicatenibacter saccharivorans, Lachnospira pectinoschiza, Lachnospiraceae bacterium and Roseburia faecis.

Deconvolution of the co-abundance network of MGS within a second independent cohort composed of 148 patients with NSCLC validated the composition of SIG1 and SIG2 of the discovery cohort, and their clinical significance.

Further investigating in other cohorts, the inventors found additional species in both SIG1 and SIG2, and minimized the weight of species previously classified in SIG1 or SIG2 (nb. no bacterial species moved from SIG1 to SIG2 or vice-versa), leading to 37 harmful (SIG1) and 45 beneficial (SIG2) bacteria.

The 37 harmful bacterial species of SIG1 are Veillonella atypica, Erysipelatoclostridium ramosum, Enterocloster bolteae, Enterocloster aldensis, Alloscardovia omnicolens, Bifidobacterium dentium, Campylobacter concisus, Clostridium perfringens, Lacticaseibacillus paracasei, Lactobacillus gasseri, Lactobacillus vaginalis, Ligilactobacillus salivarius, Limosilactobacillus fermentum, Limosilactobacillus oris, Megasphaera micronuciformis, Streptococcus anginosus, Streptococcus gordonii, Streptococcus mutans, Streptococcus parasanguinis, Streptococcus salivarius, Veillonella dispar, Veillonella parvula, Actinomyces graevenitzii, Anaerostipes caccae, Blautia producta, Campylobacter gracilis, Clostridium innocuum, Clostridium scindens, Clostridium symbiosum, Collinsella SGB14754, Enorma massiliensis, Enterocloster clostridioformis, Fournierella massiliensis, Granulicatella adiacens, Hungatella hathewayi, Proteus mirabilis and Streptococcus oralis.

The 45 beneficial bacterial species of SIG2 are Dorea formicigenerans, Dorea longicatena, Eubacterium ventriosum, Eubacterium rectale, Anaerostipes hadrus, Blautia wexlerae, Faecalibacterium prausnitzii, Roseburia hominis, Roseburia intestinalis, Roseburia inulinivorans, Ruminococcus bicirculans, Ruminococcus lactaris, Candidatus Cibiobacter qucibialis, Clostridiales bacterium KLE1615, Faecalibacillus intestinalis, Lachnospira eligens, Agathobaculum butyriciproducens, Anaerobutyricum hallii, Blautia massiliensis, Clostridiaceae bacterium, Clostridium sp AF34 10BH, Lachnospira pectinoschiza, Anaerotignum faecicola, Clostridiaceae bacterium OM08 6BH, Clostridiaceae unclassified SGB4769, Clostridiales unclassified SGB15145, Clostridium fessum, Clostridium sp AM22 11AC, Clostridium sp AM33 3, Clostridium sp AM49 4BH, Coprobacter fastidiosus, Coprococcus comes, Coprococcus eutactus, Eubacterium ramulus, Faecalibacterium SGB15346, Firmicutes bacterium AF16 15, Gemmiger formicilis, Lachnospira sp NSJ 43, Lachnospiraceae bacterium OM04 12BH, Lachnospiraceae bacterium WCA3 601 WT 6H, Lacrimispora amygdalina, Mediterraneibacter butyricigenes, Oscillibacter sp ER4, Phocaeicola massiliensis and Roseburia sp AF02 12.

The inventors demonstrated that a value calculated from the numbers of bacteria from SIG1 and SIG2 present in an individual'≤gut microbiota allowed, when it is in certain ranges, estimation of the likelihood that the individual has an intestinal dysbiosis. Subsets of the above SIG1 and SIG2 could also be successfully used for this purpose. This value can be calculated either as a normalized ratio of bacteria from SIG1 and SIG2 present in a sample from the individual, or as a normalized difference between these.

According to a first aspect, the present invention thus pertains to a method of diagnosing intestinal dysbiosis in an individual, comprising:
(i) in a sample from said individual comprising intestinal microbiota, assessing the presence or absence of bacterial species of a first species interacting group (“SIG1”) consisting of N₁ bacterial species comprising at least 5, preferably at least 6, more preferably at least 7 bacterial species selected from a group of bacterial species identified above as harmful bacteria;
(ii) in a sample from said individual comprising intestinal microbiota, assessing the presence or absence of bacterial species of a second species interacting group (“SIG2”) consisting of N₂ bacterial species comprising at least 5, 6 or 7, preferably at least 10 to 12, more preferably at least 14 bacterial species selected from a group of bacterial species identified above as beneficial bacteria; NSG1
(iii) calculating a FRNormCount as follows:

$$FRNormCount = \frac{\frac{{N}_{SIG 1}}{N 1}}{\frac{{N}_{SIG 2}}{N 2}} ,$$

wherein N_{SIG1} is the number of bacterial species of SIG1 present in the sample and N_{SG2} is the number of bacterial species of SIG2 present in the sample; and/or
(iv) calculating a S score as follows:

$$S = ( \frac{{N}_{SIG 2}}{N 2} - \frac{{N}_{SIG 1}}{N 1} - 1 ) / 2$$

wherein N_{SG1} is the number of bacterial species of SIG1 present in the sample and N_{SG2} is the number of bacterial species of SIG2 present in the sample.

From the “FRNormCount” and/or “S score” calculated in steps (iii) and/or (iv) above, a TOPOSCORE, reflecting the likelihood that the individual has a dysbiosis, is defined as follows: if the FRNormCount is inferior to a first predetermined threshold TOPO₁ and/or if the S score is superior to a predetermined threshold S₂, the individual is likely not to have intestinal dysbiosis (TOPOSCORE=1), and if the FRNormCount is superior to a second predetermined threshold TOPO₂ superior to TOPO₁ and/or if the S score is inferior to a predetermined threshold S₁ inferior to S₂, the individual is likely to have intestinal dysbiosis (TOPOSCORE=5).

Individuals with a score falling into an intermediate category (“Grey zone”, neither SIG1 nor SIG2) could be further segregated based on the relative abundance of Akkermansia spp., as previously described (Derosa et al. 2021; WO 2022/157207). Combining the SIG1/SIG2 ratio (FRNormCount) or the SIG2-SIG1 difference (S score) and Akkermansia spp (Akk) relative abundance led to the “TOPOSCORE”, allowing estimation of the likelihood of an individual of having a dysbiosis. Noticeably, this TOPOSCORE also enabled to estimate the likelihood of an individual of responding to ICI with a superior accuracy than PD-L1 expression or International Metastatic RCC Database Consortium (IMDC) risk score, in a third independent and prospective cohort of 61 NSCLC and 83 kidney cancer patients amenable to PD1 blockade respectively (Example 3 below), as well as in B-cell lymphoma (Example 8), urothelial cancer (Example 9) and colorectal cancer (example 14).

Hence, according to a preferred embodiment, in cases where TOPO₁≤FRNormCount≤TOPO₂ and/or S₁≤S≤S₂ (“grey zone”), the above method further comprises a step of measuring the relative abundance of bacteria of the Akkermansia genus (Akk) in a fecal material sample from said individual, wherein:
a) if Akkermansia bacteria are present in the sample below a predetermined threshold (“Akk superior threshold”), the patient is likely not to have intestinal dysbiosis (TOPOSCORE=2); and
b) if no Akkermansia is present in the sample, the individual is likely to have intestinal dysbiosis (TOPOSCORE=3);
c) if Akkermansia bacteria are present in the sample above the Akk superior threshold, the individual is likely to have intestinal dysbiosis (TOPOSCORE=4).

The present invention also pertains to the use of the TOPOSCORE as a theranostic tool for determining if a patient having a cancer amenable to immune-oncology (I-O) therapy is likely to be a good responder to said therapy and/or if the patient needs a microbiota-centered intervention (MCI) before administration of said I-O therapy, wherein the higher the patient'≤TOPOSCORE, the lower the probability that the patient responds to said I-O therapy in absence of a MCI before or along with said I-O therapy. In particular, a TOPOSCORE 3 indicates that the individual needs an MCI. This aspect of the invention is important to exclude a patient likely to have a primary resistance to an I-O therapy due to intestinal dysbiosis from a treatment with said I-O, to avoid deleterious side-effects not accompanied by any therapeutic effect.

According to the invention, the TOPOSCORE can also be used as a pharmacodynamics tool to monitor the evolution of the intestinal microbiota of an individual receiving an MCI and/or a treatment possibly impacting the intestinal microbiota and/or impacted by the intestinal microbiota. This aspect of the invention is important to quickly identify situations where a patient develops a secondary resistance to an I-O therapy due to intestinal dysbiosis. The treatment is then discontinued or combined with a MCI to restore the response thereto.

The invention also relates to the use of the TOPOSCORE for assessing whether a fecal material can be used in an MCI, wherein if the TOPOSCORE≥3, the fecal material cannot be used in an MCI and if the TOPOSCORE≤2, preferably if FRNormCount=0, the fecal material can be used in an MCI.

As shown in Example 6 below, the inventors also simplified its calculation using restricted amount of MGS and using a PCR-based user-friendly test. By converting the TOPOSCORE to a PCR-based test with a rapid turnaround time, it will be possible to adopt this score in routine clinical testing to improve patient stratification and ICI success rates.

Accordingly, a kit of parts for determining the TOPOSCORE from a sample, comprising a primer pair and/or a nucleic acid probe specific for each of the bacterial species the presence of which is to be assessed to calculate the TOPOSCORE, is also part of the present invention.

### BRIEF DESCRIPTION OF THE DRAWINGS

FIG. 1. Classical method to estimate the performance of MGS in predicting clinical benefit to ICI. A-B. Taxonomic alpha-diversity of patient samples was estimated using the Shannon Diversity Index (upper panel) calculated as

$$H = - {\sum }_{i = 1}^{k} □ PilogPi ,$$

where k is the total number of species within the sample (richness), and P is the proportion of k made up of the i-th species. The H index was computed for non-responders (NR) and responders (R) patients with follow-up >12 months from the discovery cohort (A, n=245) and the validation cohort (B, n=148). Beta-diversity (Principal Coordinate Analysis, PCoA, middle panel) of fecal microbiota (microbial relative abundance) according to patient subgroups [light grey: NR (OS<12 months), black: R (OS>12 months)] in patients with non-small cell lung cancer (NSCLC) treated with anti-PD-1/PD-L1 antibodies. We implemented Partial Least Square Discriminant Analysis (PLS-DA) and the subsequent Variable Importance Plot (VIP) as a supervised analysis in order to identify the most discriminant microbial species among the two patient groups R vs NR (right panel). ANOSIM and PERMANOVA define the separation of the groups; p values define the significance of group separation after 999 permutations of the samples. Mann-Whitney U test p-values (*p<0.05,**p<0.01,***p<0.001) are indicated. C. Cox regression univariate analysis and Kaplan Meier curves of overall survival (OS) of NSCLC patients from the validation cohort according to the “high”≥0 and the “low”<0 of normalized and standardized relative abundance values as cut-off values of Anaerostipes hadrus and Roseburia intestinalis monitored by MGS in fecal samples at baseline. Also refer to Table 2 for patient characteristics and FIG. 2 for the same analyses in the discovery cohort. D. ROC curves and AUC measuring the performance of the relative abundance of most significant MGS retained in machine learning algorithms using the discovery and validation cohorts to predict clinical benefit to ICI, using MetaPhlAn4 pipeline. E. Idem as in C. right in the renal cell carcinoma (RCC) cohort (Table 2). F-G. Actualized Discovery (F) and Validation (G) cohorts (2023) investigating alpha and beta-diversities of the stool metagenomic composition. Taxonomic alpha-diversities (insets) of patient samples were estimated using the Shannon Diversity Index calculated as

$$H = - {\sum }_{i = 1}^{k} □ PilogPi ,$$

where k is the total number of species within the sample (richness), and P is the proportion of k made up of the i-th species. The H index was computed for non-responders (OS<12 months) and responders (OS>12) patients with follow-up >12 months from the discovery cohort (A) and the validation cohort (B). Beta-diversities (Principal Coordinate Analysis, PCoA, middle panel) of fecal microbiota (microbial relative abundances) according to patient subgroups [orange: OS<12 months, blue: OS>12 months]. Supervised analysis using Partial Least Square Discriminant Analysis (PLS-DA) and Variable Importance Plot (VIP) to identify the most discriminant microbial species among the two patient groups. ANOSIM and PERMANOVA define the separation of the groups; p values define the significance of group separation after 999 permutations of the samples. Mann-Whitney U test p-values (*p<0.05, **p<0.01, ***p<0.001) are indicated.

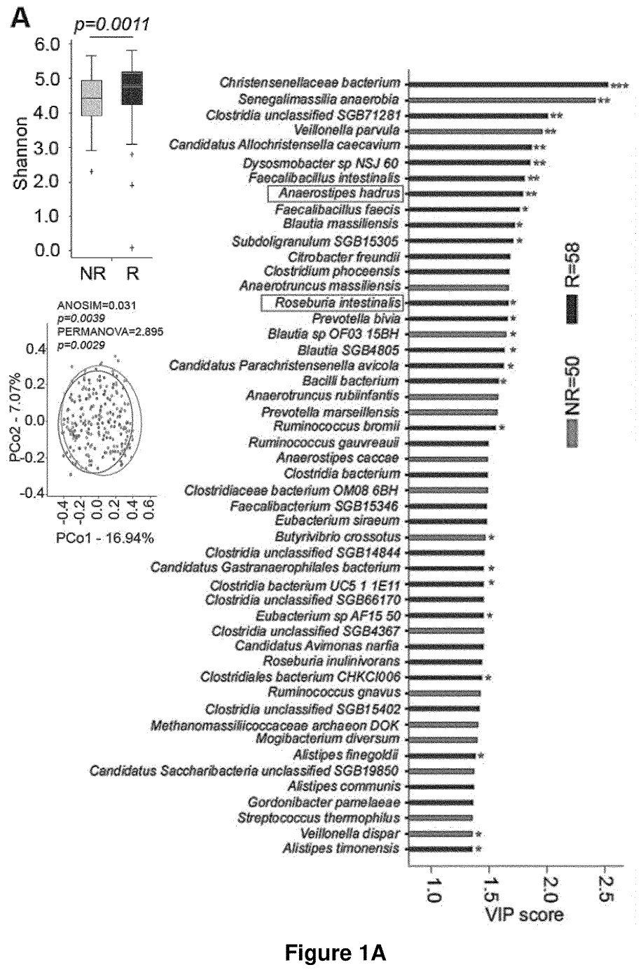

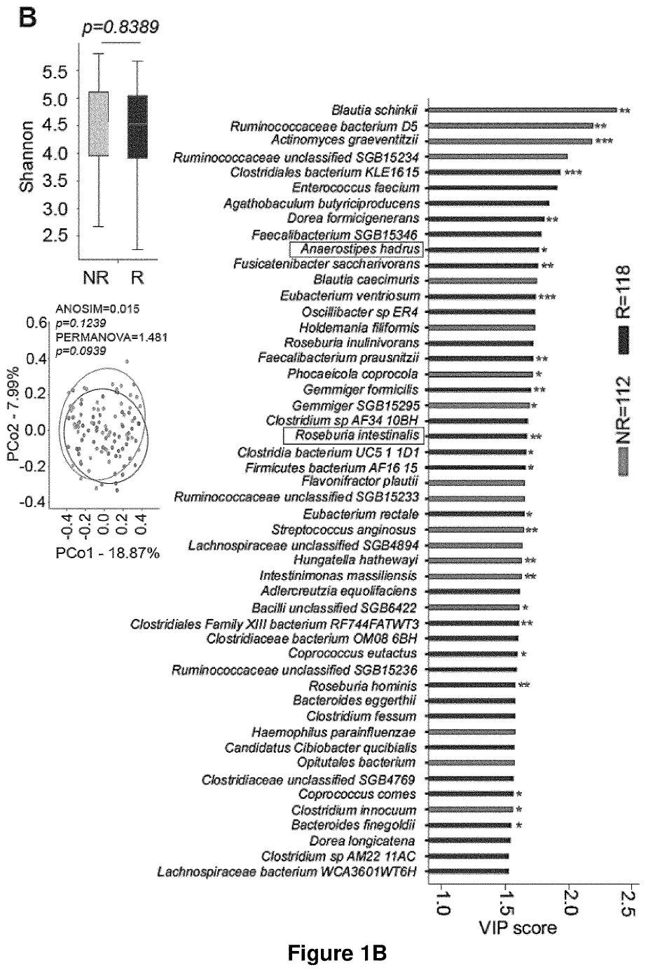

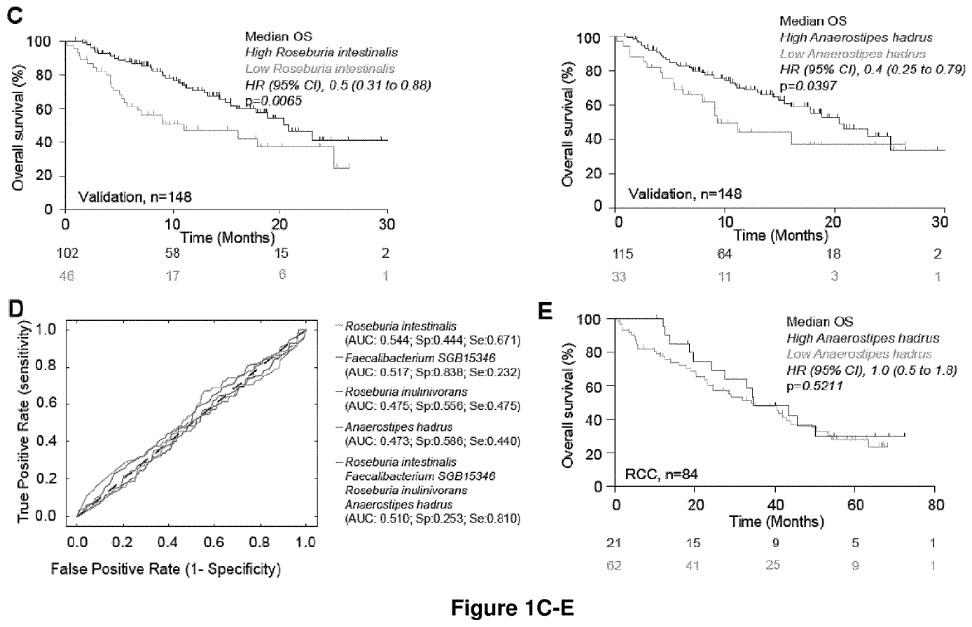

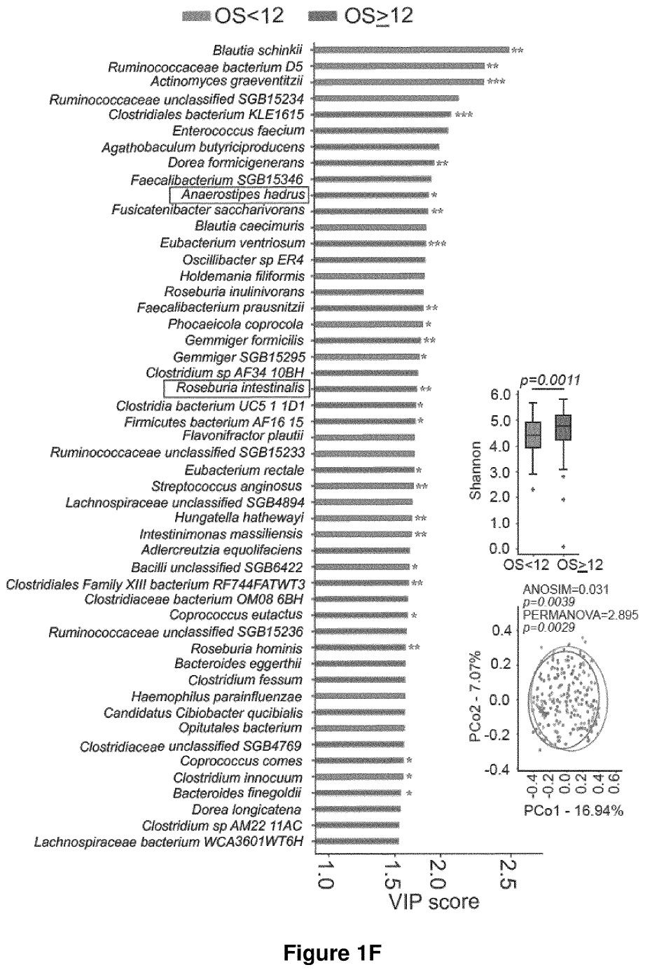

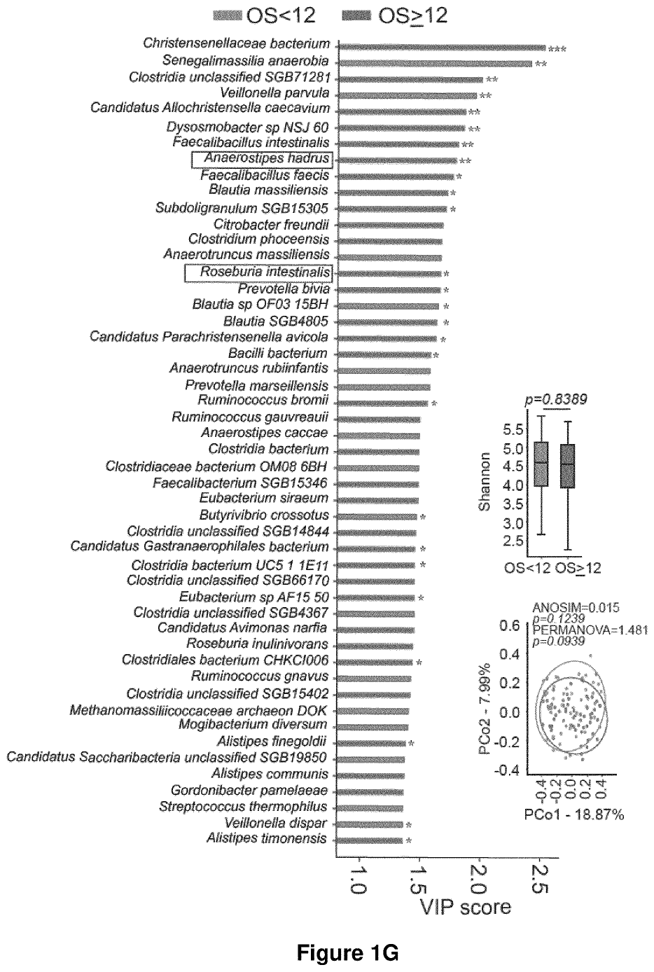

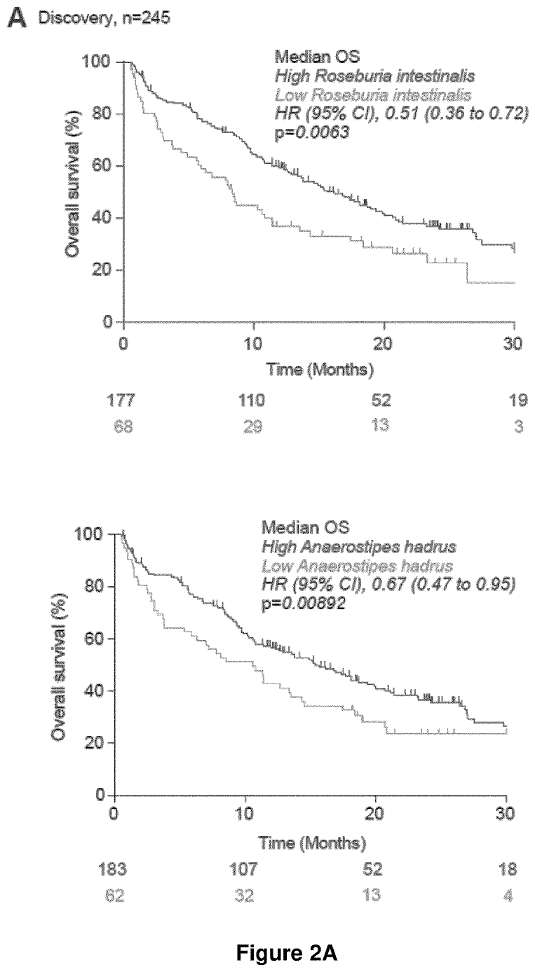

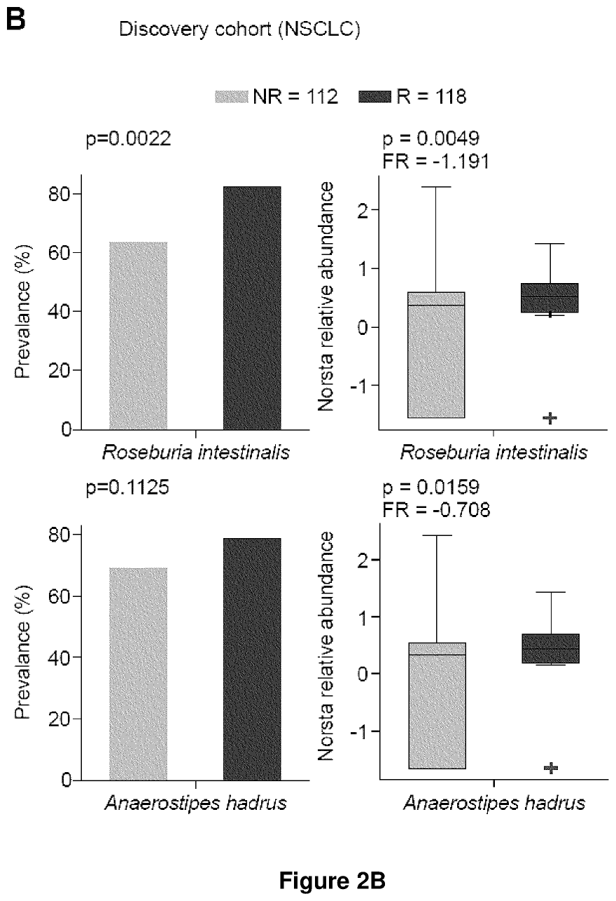

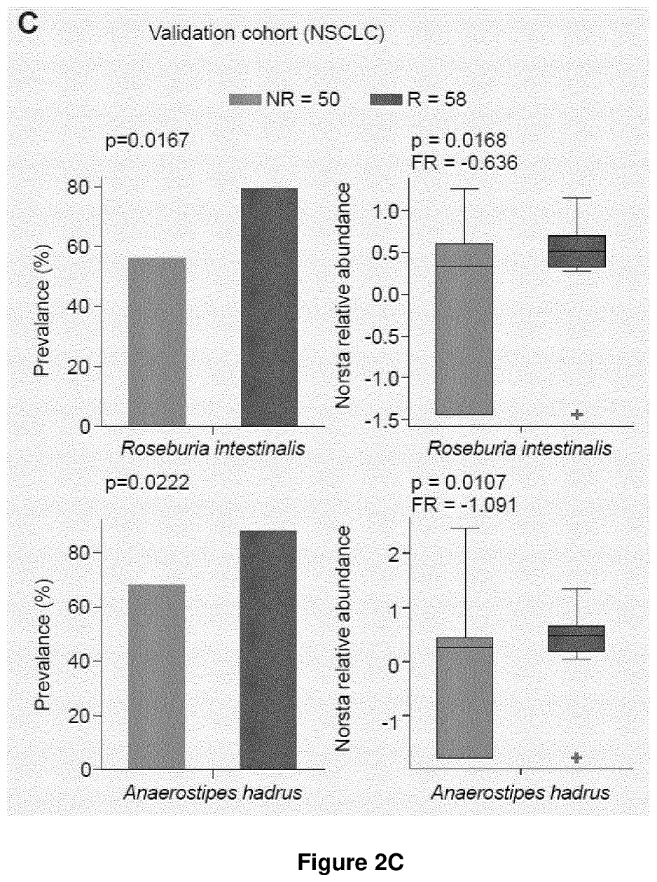

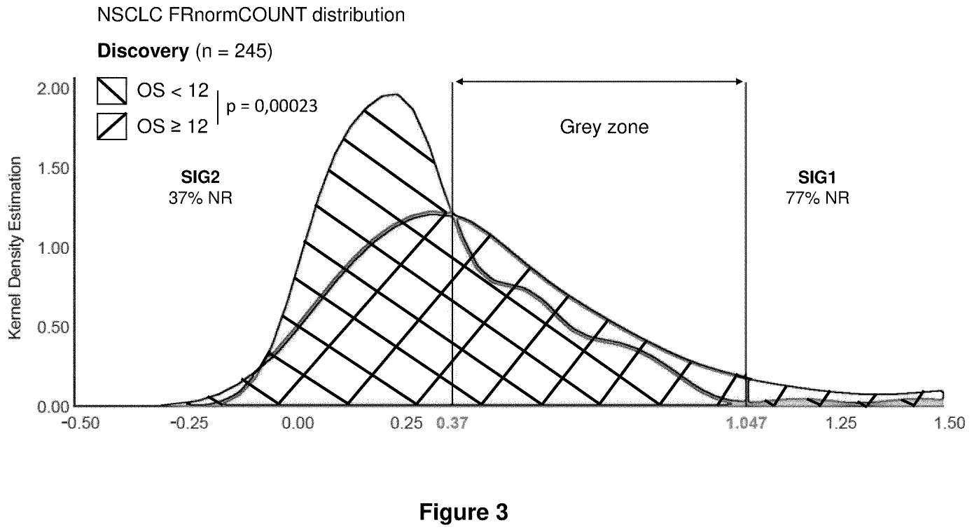

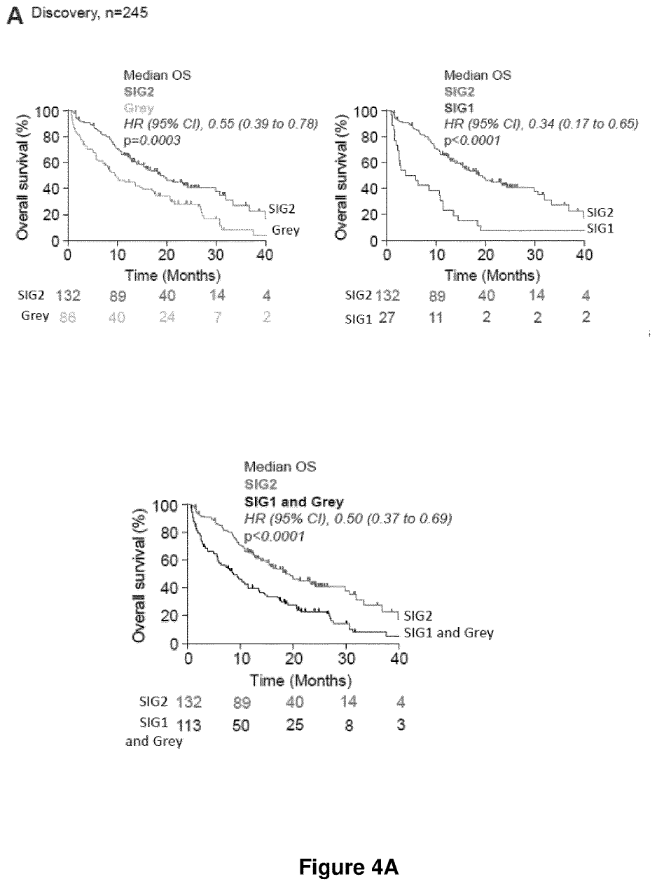

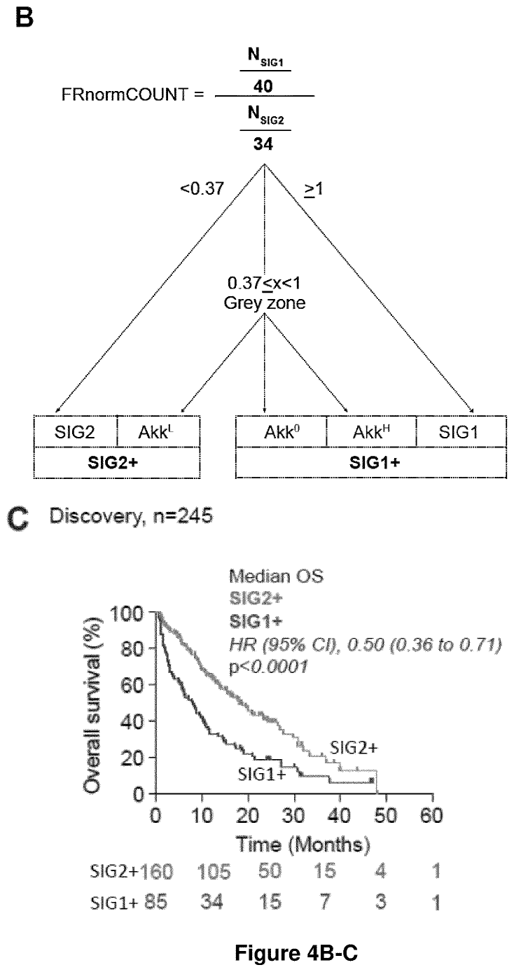

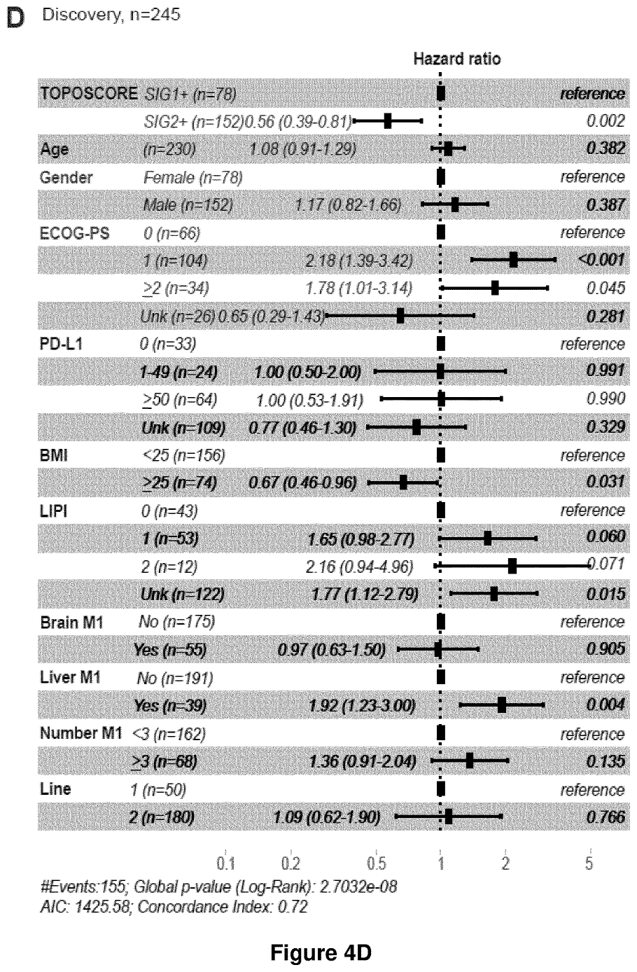

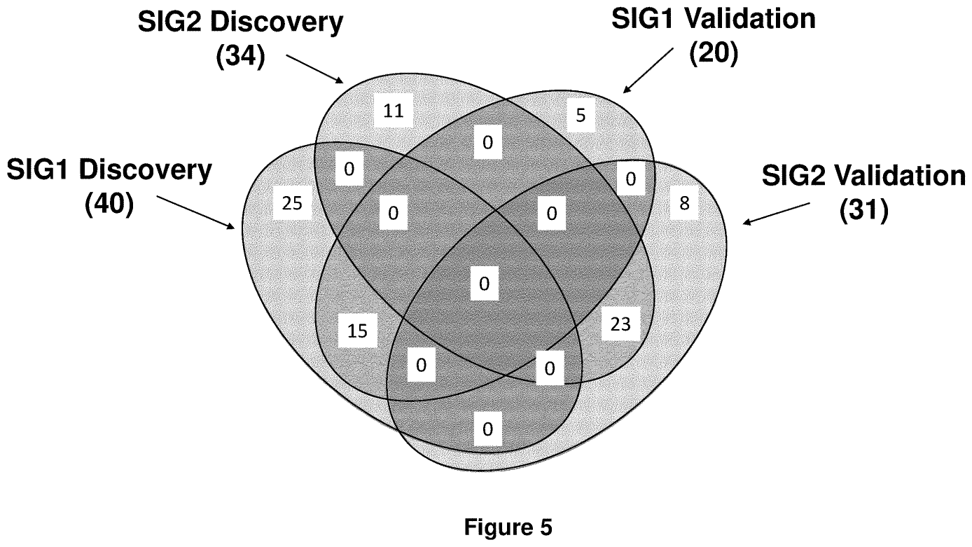

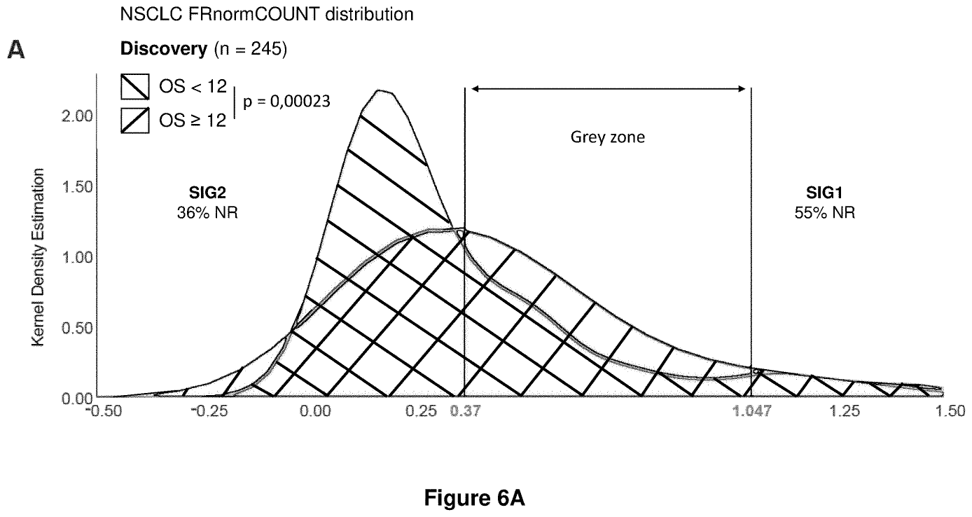

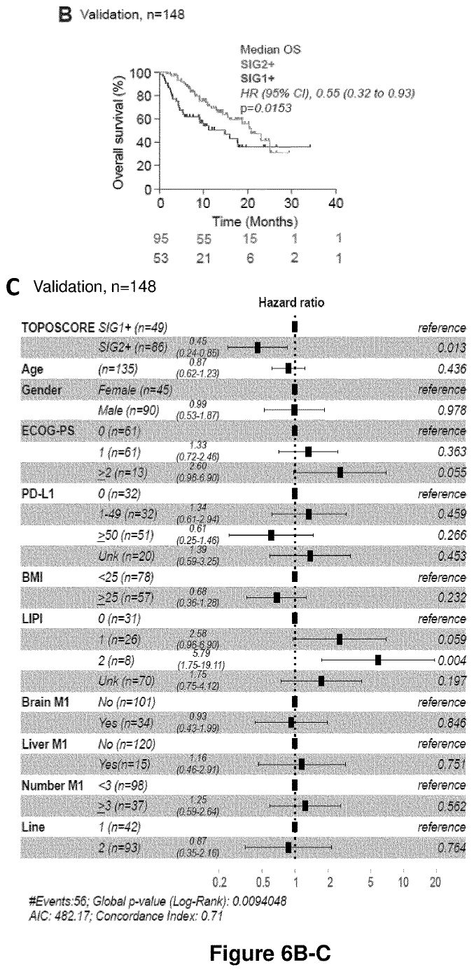

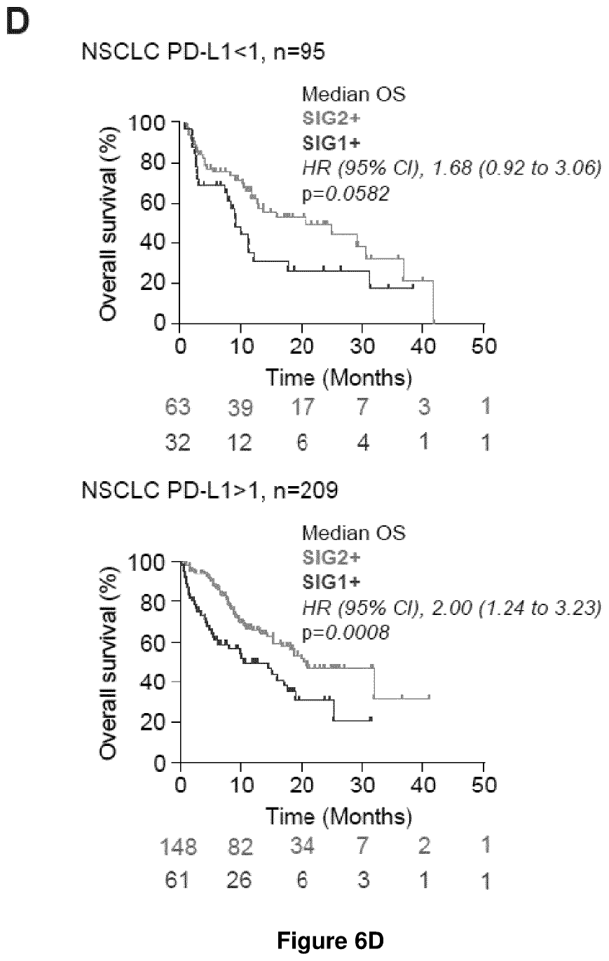

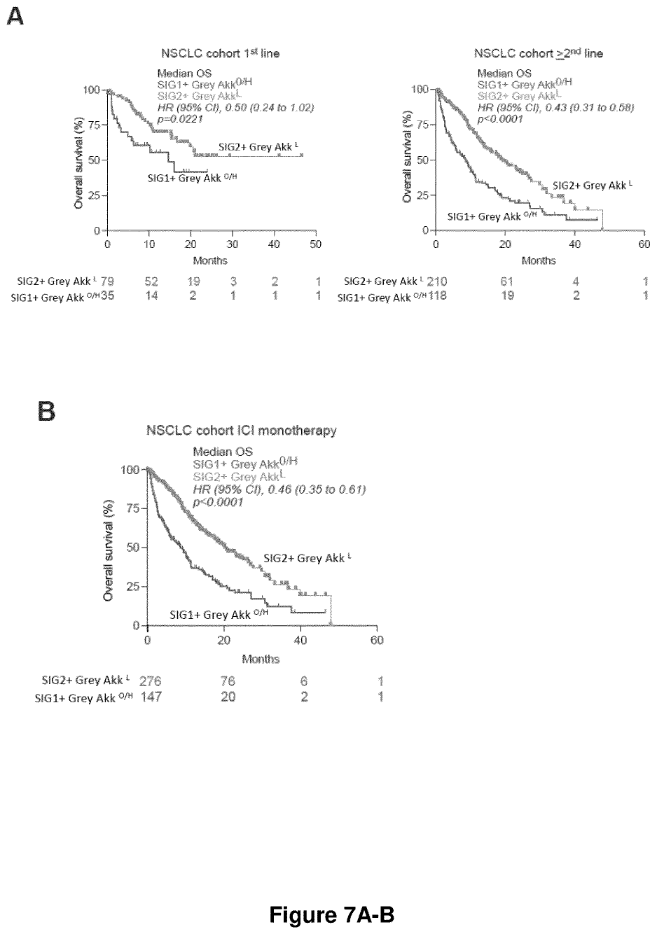

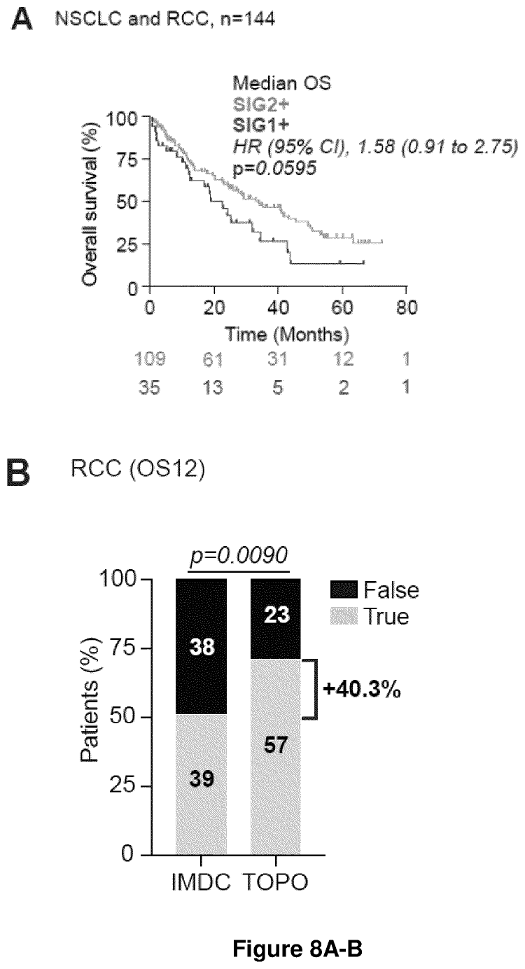

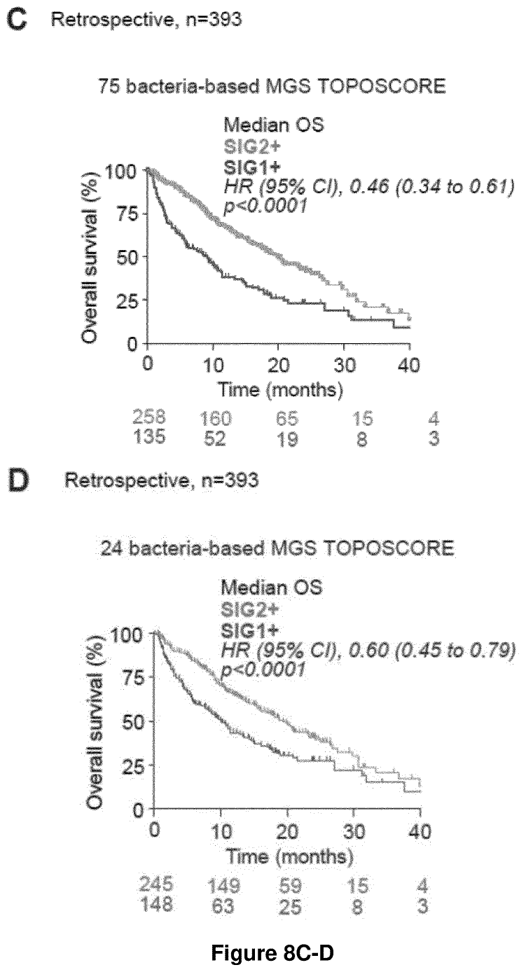

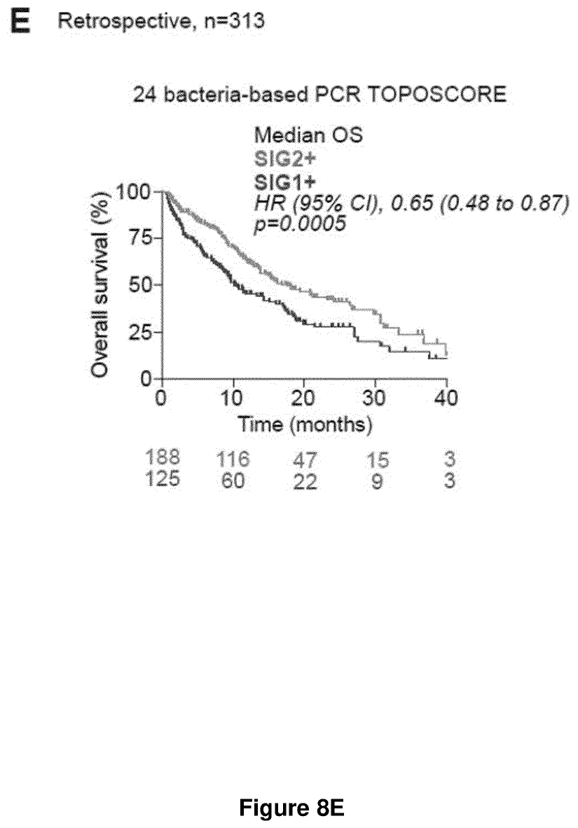

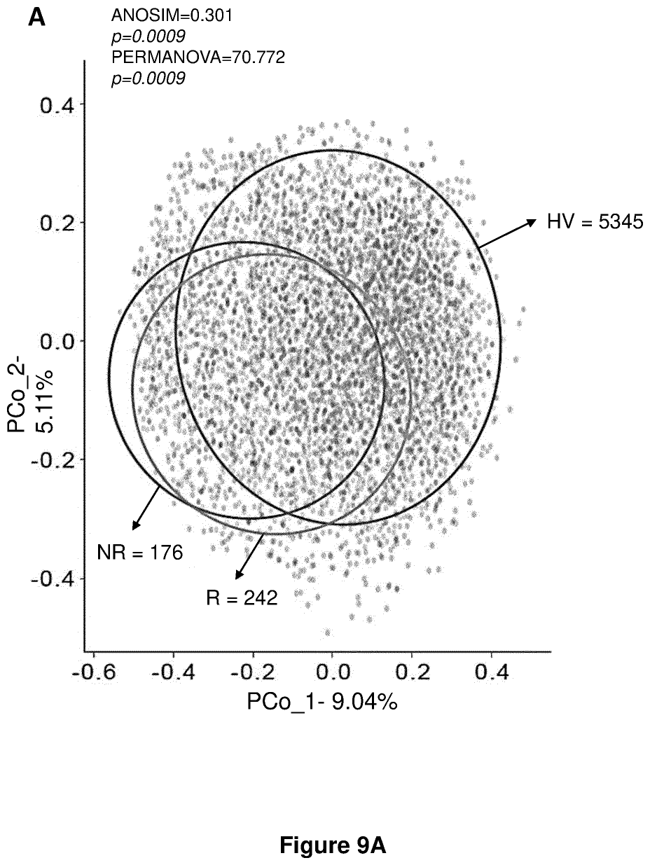

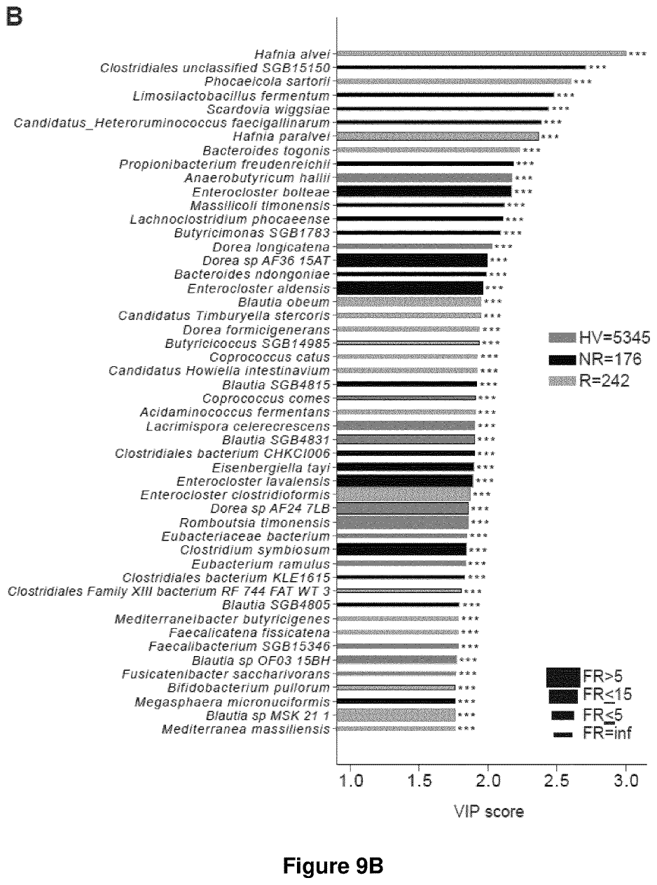

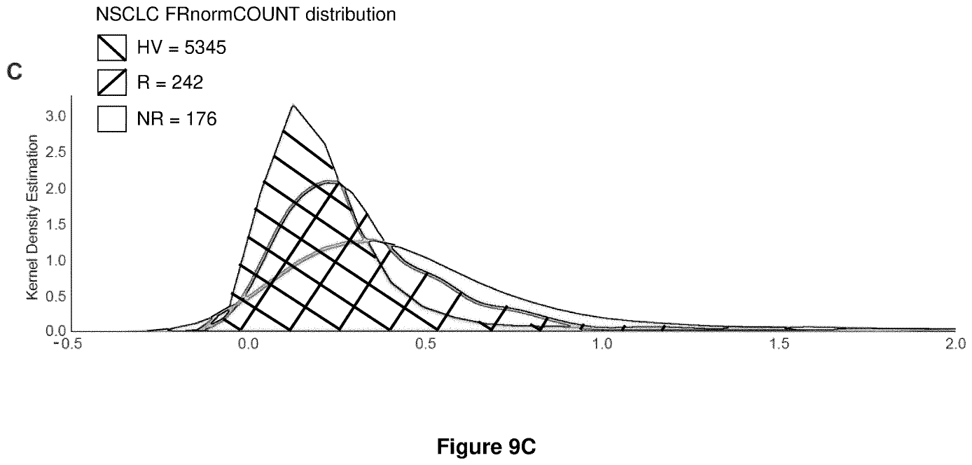

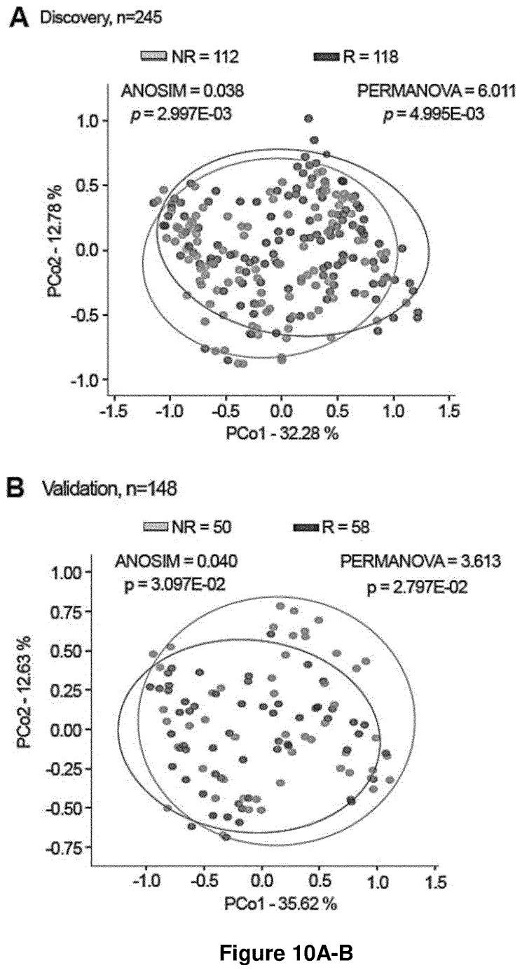

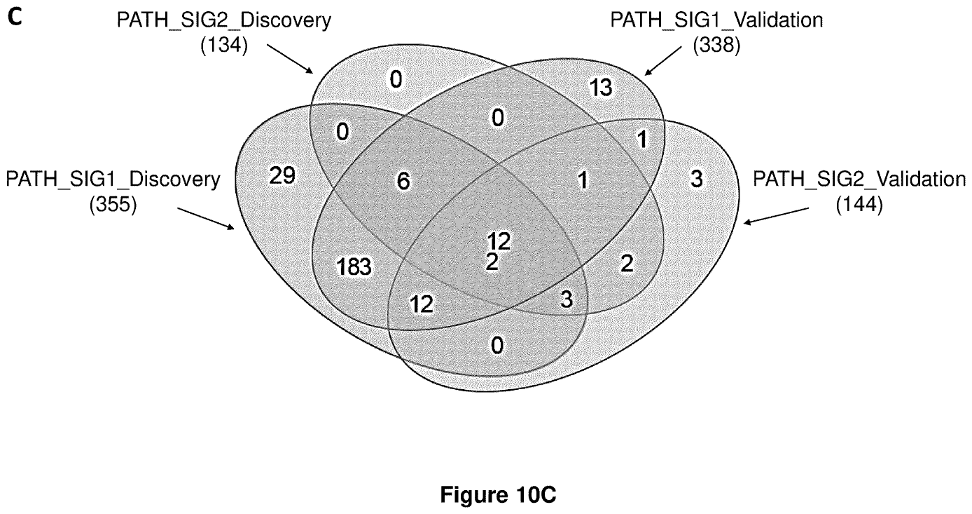

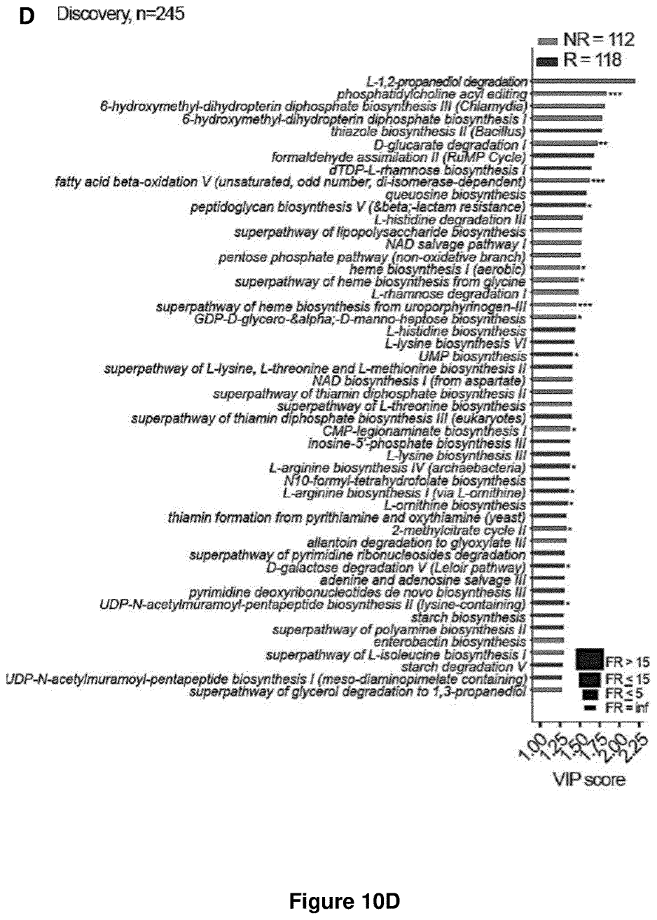

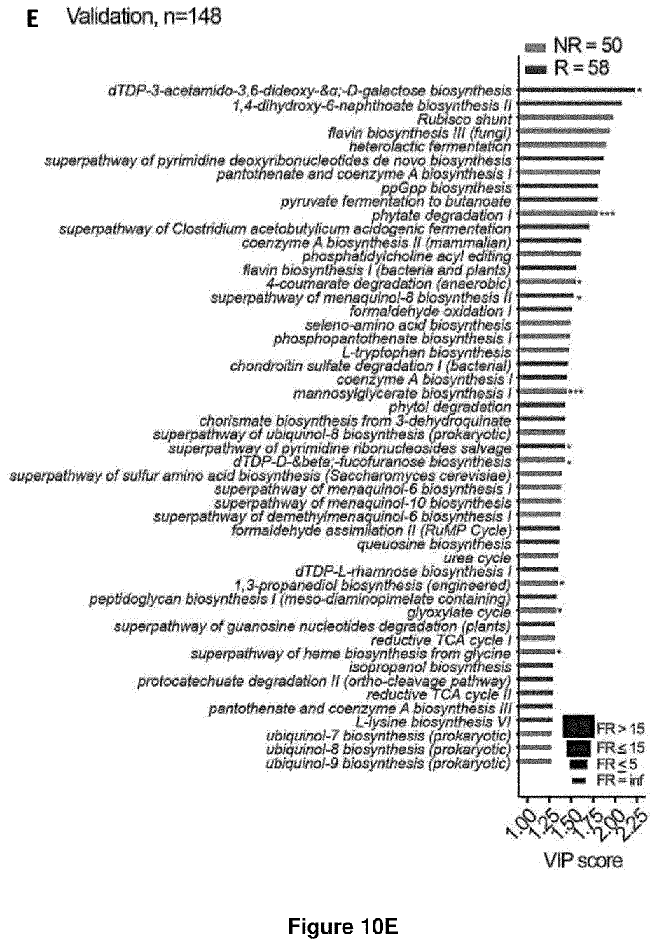

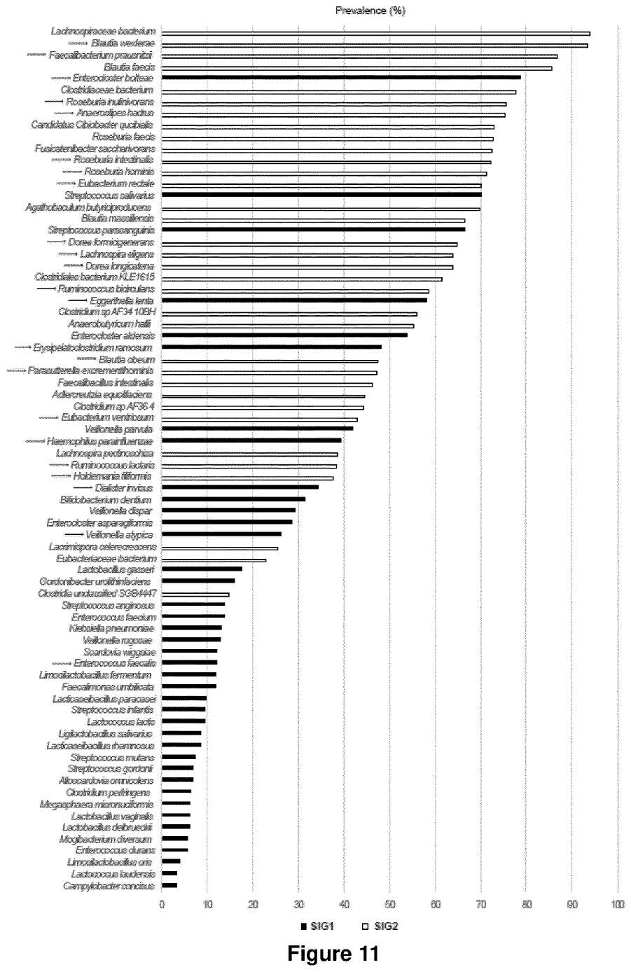

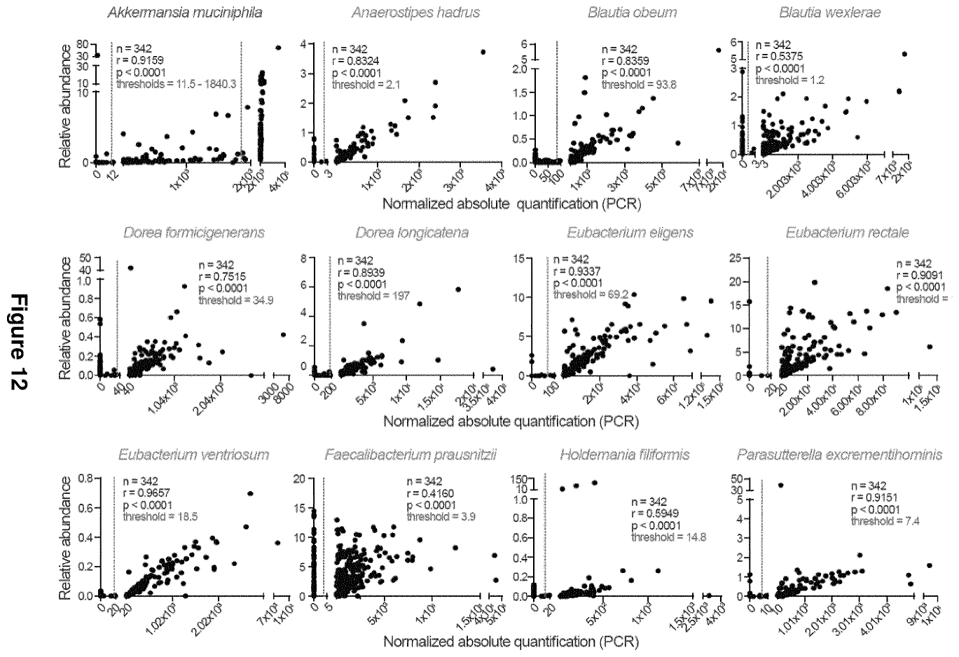

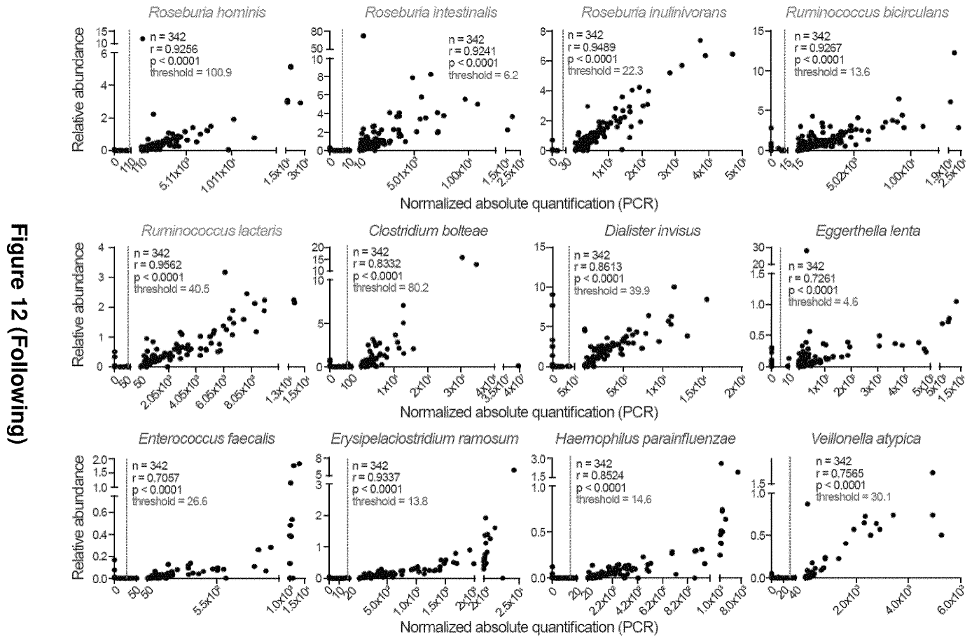

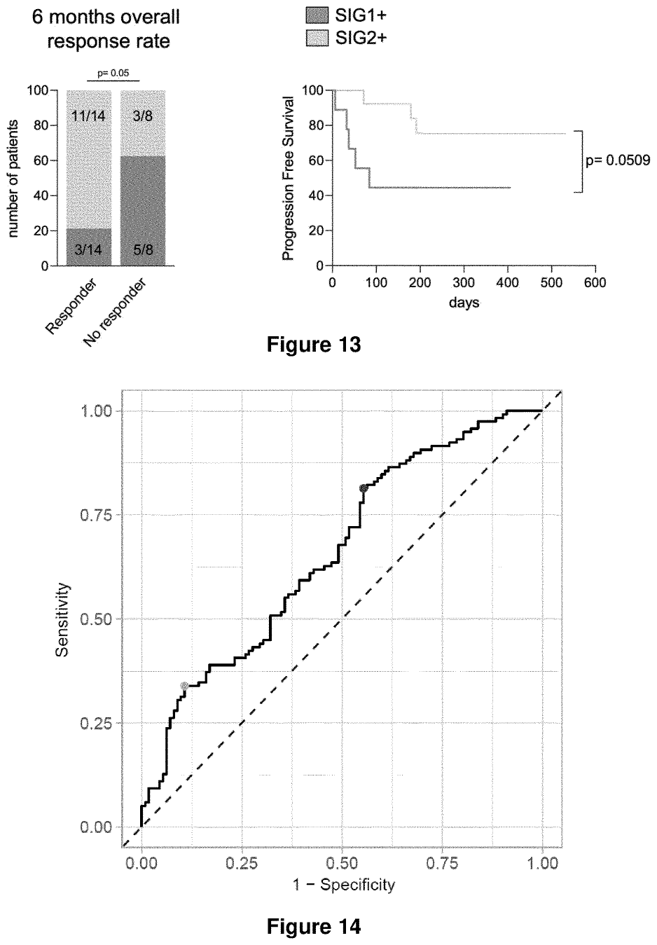

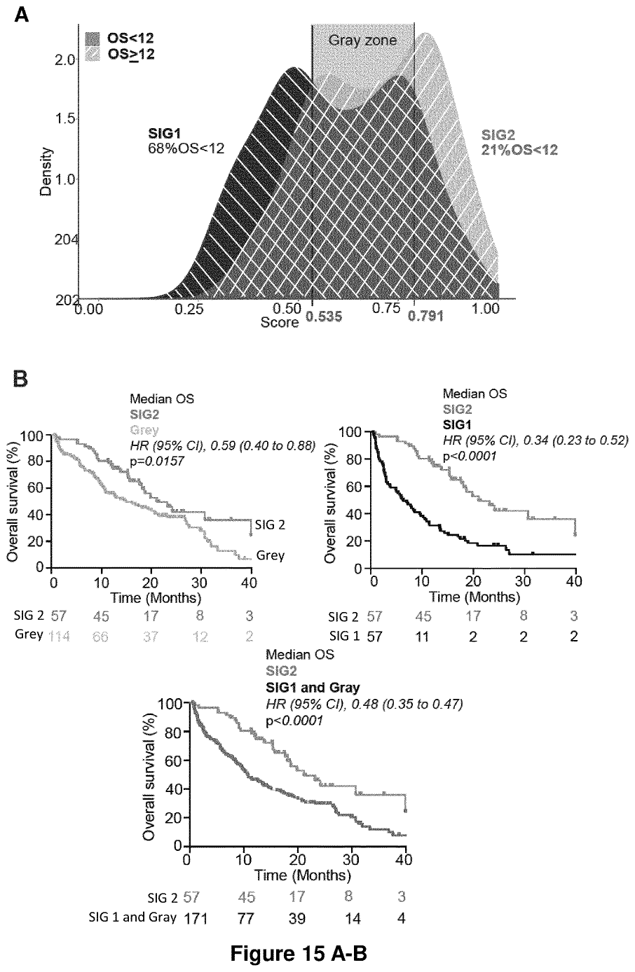

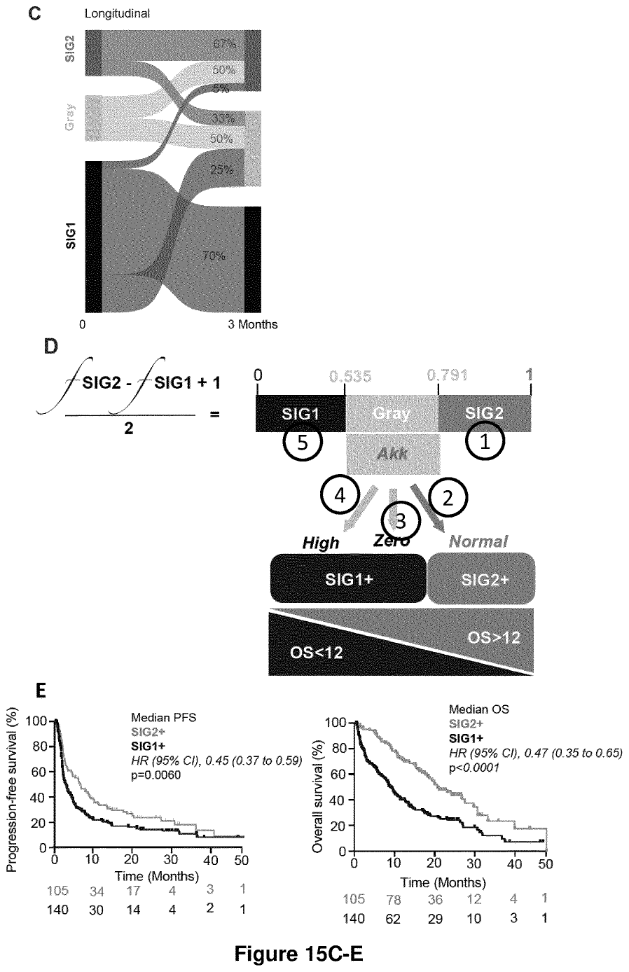

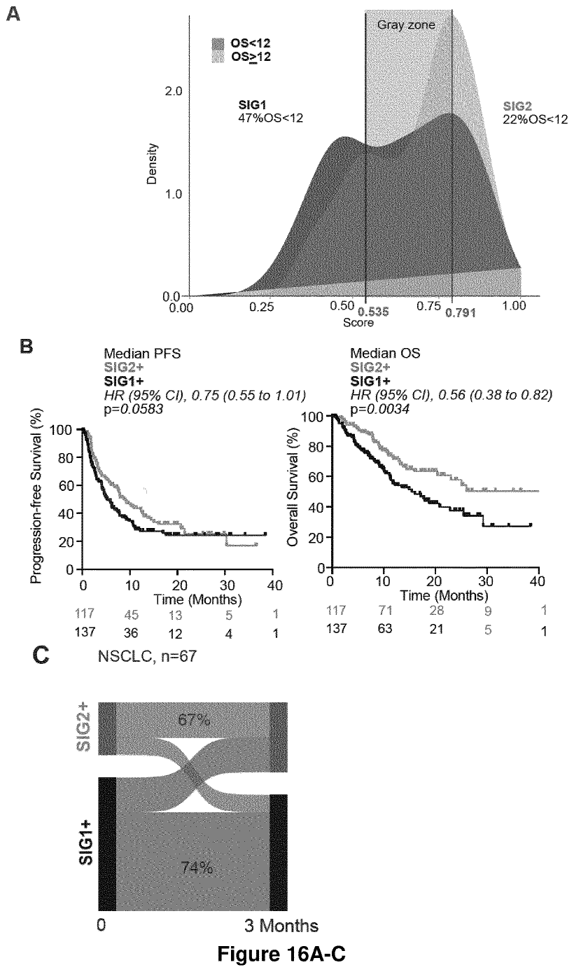

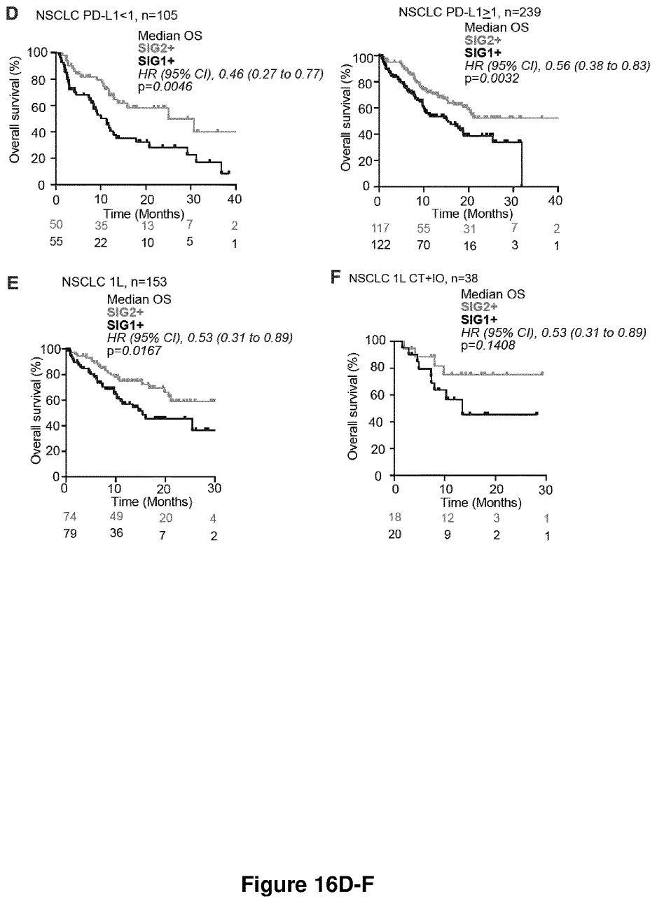

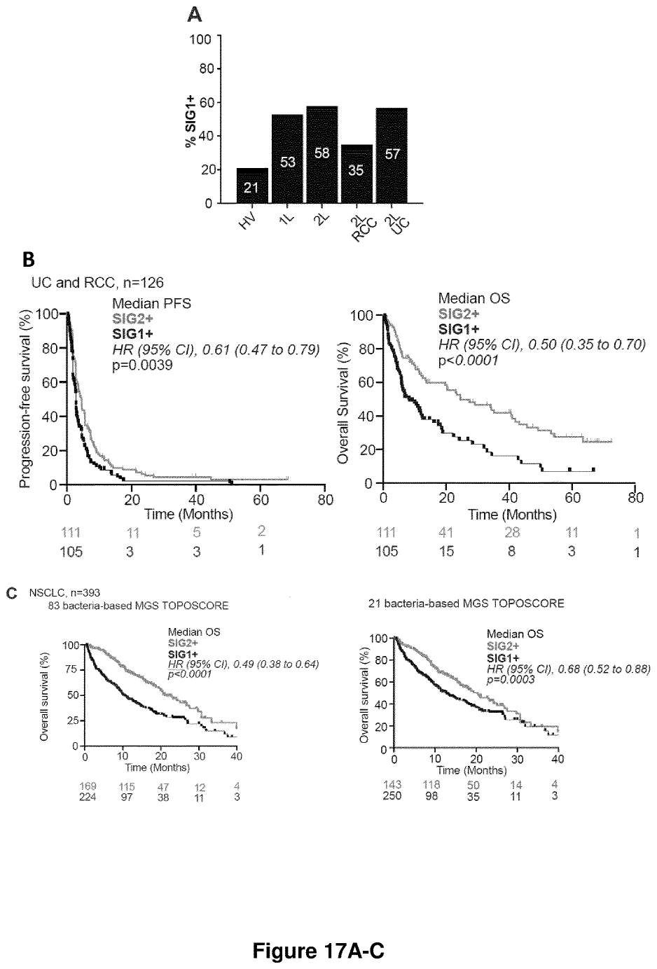

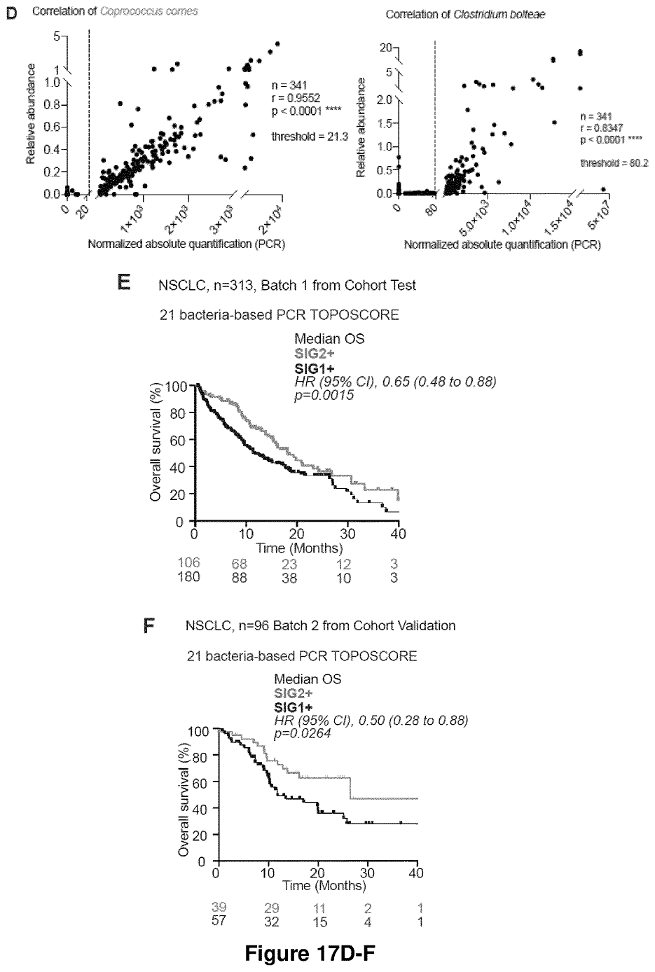

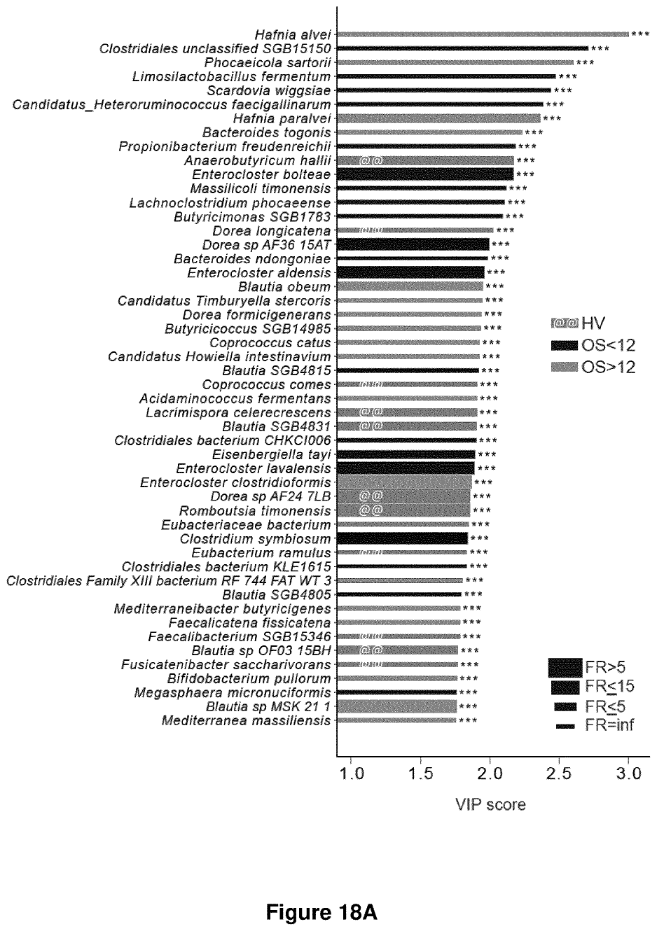

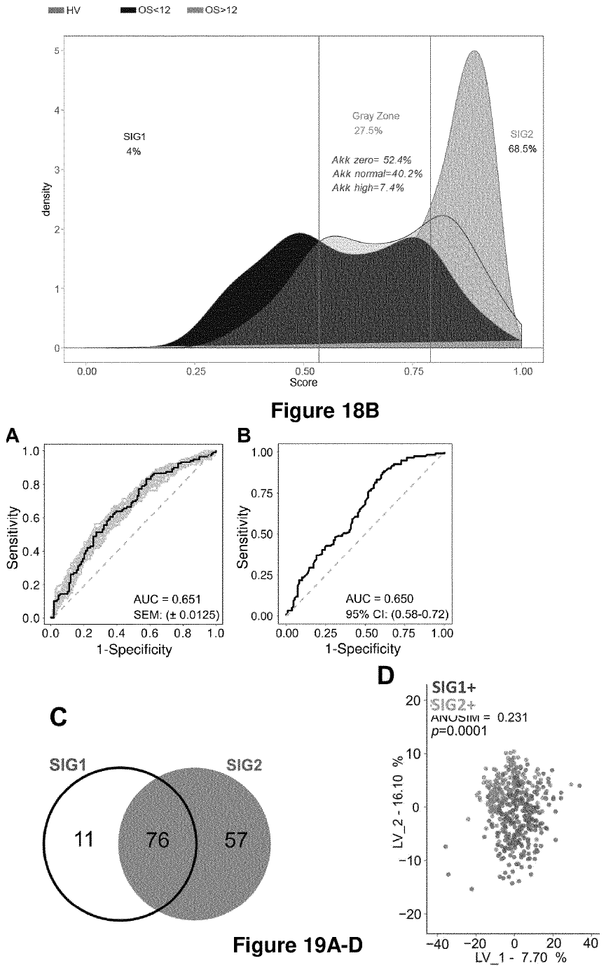

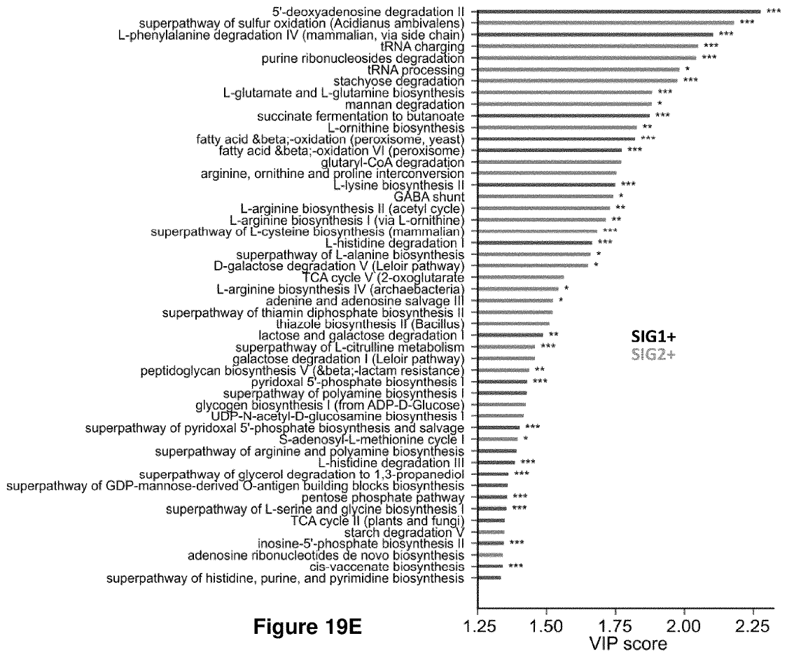

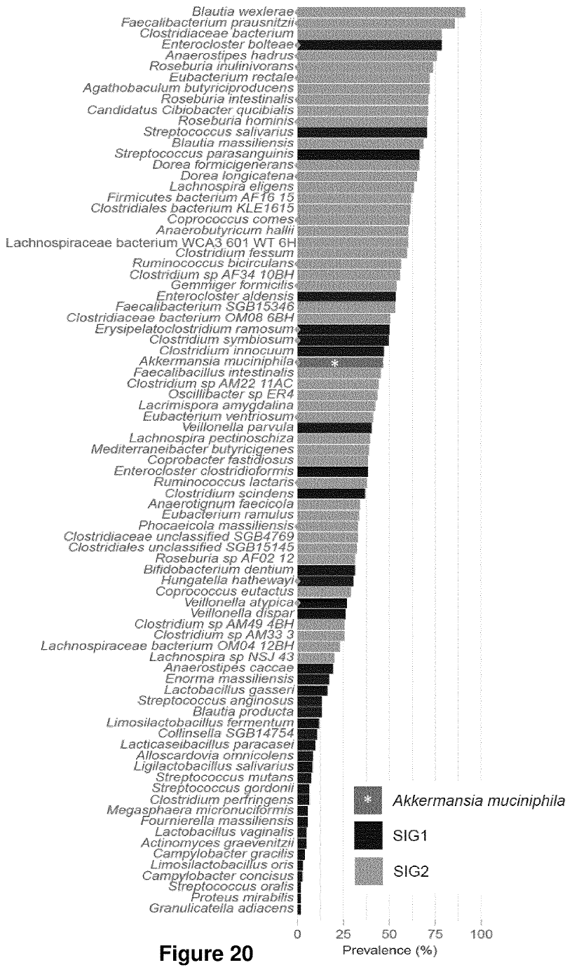

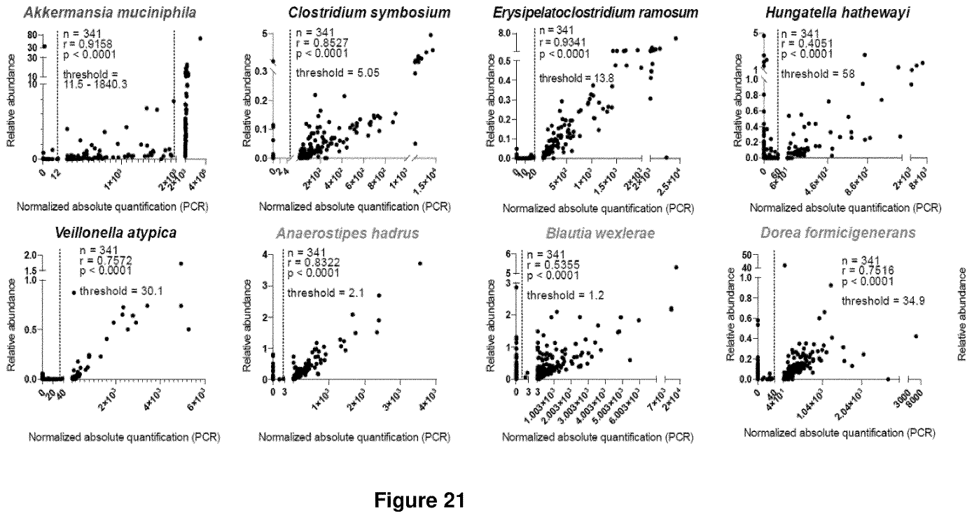

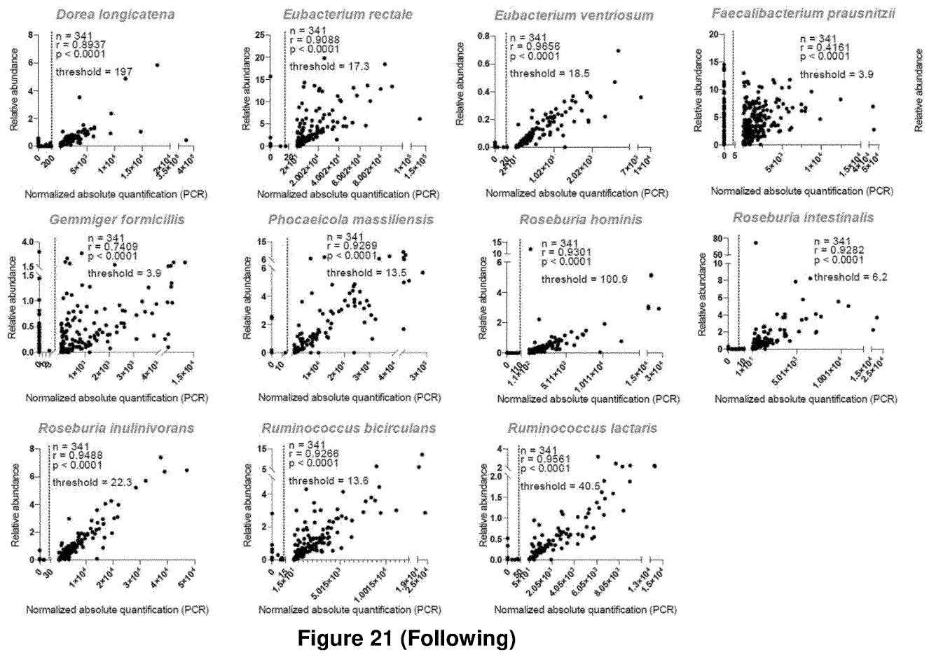

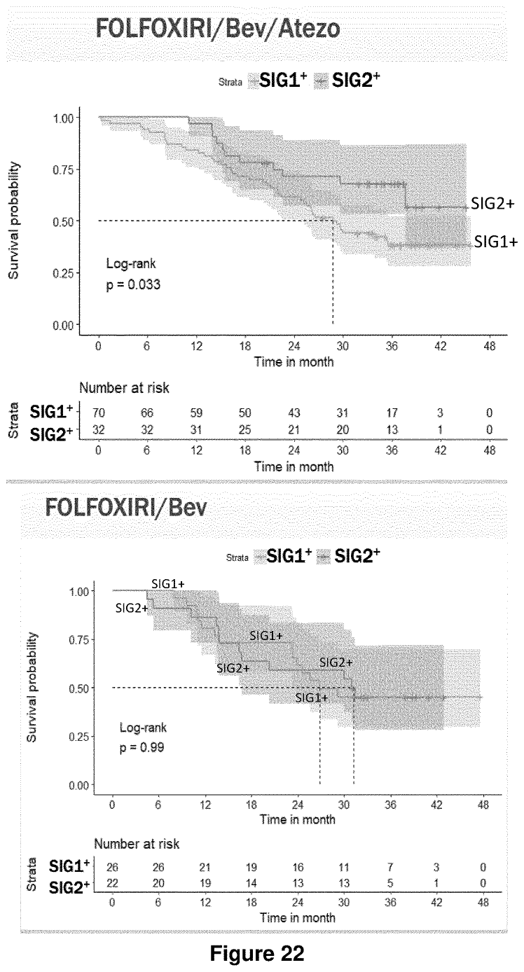

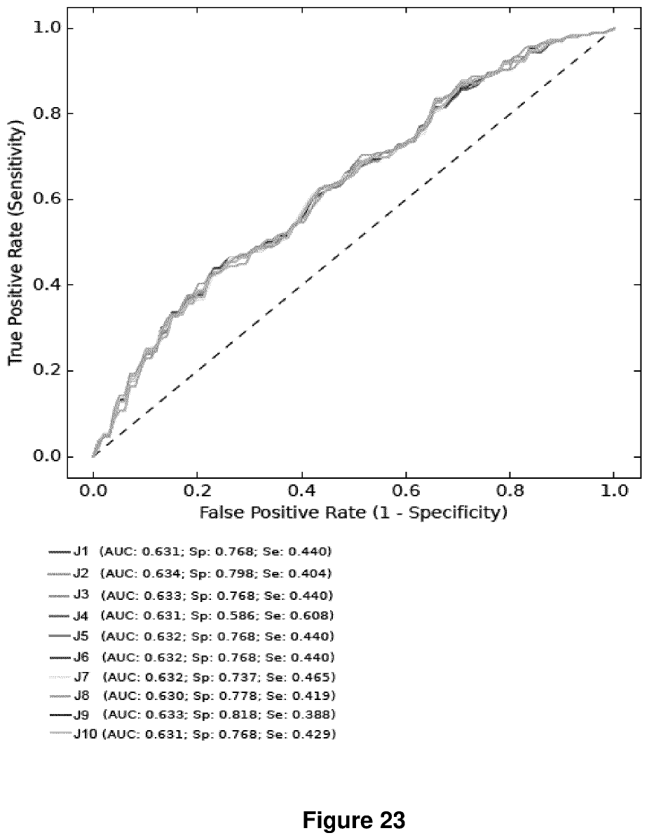

FIG. 2. Most significant MGS species are associated with clinical benefit to ICI. A. Cox regression univariate analysis and Kaplan Meier curves of overall survival (OS) of NSCLC patients from the discovery cohort according to “high” _0 and the “low”<0 of normalized and standardized relative abundance values as cut-off values of Roseburia intestinalis (upper panel) and Anaerostipes hadrus (lower panel), monitored by MGS in fecal samples at baseline. Also refer to Table 2 for patient characteristics and FIG. 1C for the same analyses in the validation cohort. B-C. Prevalence and normalized/standardized relative abundance of the two MGS species (A. hadrus and R. intestinalis) found in common in the two cohorts (discovery in B, validation in C), colored according to patient subgroups (light grey: non-responders, NR, OS<12 months, dark grey: responders, R, OS≥12 months) in NSCLC patients treated with anti-PD-1/PD-L1 antibodies.

FIG. 3. Co-abundance Pearson networks and Species Interacting Groups (SIG) associated with response or resistance to ICI in the discovery cohort of NSCLC. The distribution of the FRnormCOUNT parameter, computed as in Materials and Methods section, is depicted in the discovery cohort of NSCLC patients by means of Kernel Density Estimation (KDE). SIG2 and SIG1 ratio distributions are depicted in oblique lines of distinct orientations and the boundaries between these two SIG are in the Y axis and individualize the Grey Zone.

FIG. 4. Design and performance of the TOPOSCORE in the discovery cohort. A. Cox regression univariate analysis and Kaplan Meier curves of overall survival (OS) for the 245 NSCLC patients according to the 3 regions within the FRnormCOUNT (1) a SIG2 region (0<x<0.37),; 2) a Grey zone (0.37≤x<1.047), 3) a SIG1 region (x≥1.047)) calculated in fecal samples at baseline (Table 2 for patients' characteristics). Of note, similar conclusions were drawn for PFS (not shown). B. Decision-making tree using FRnormCOUNT and Akkermansia spp relative abundance. Three different regions resulted for FRnormCOUNT values: 1) a SIG2 region (0<x<0.37), mostly harboring R; 2) a Grey zone (0.37≤x<1.047), in which NR and R were equally represented; 3) a SIG1 region (x≥1.047), mostly harboring NR. Patients' distribution was statistically significant as per X² statistics. Within the Grey zone, all patients harboring low A. muciniphila (Akk) levels (0<Akk≤4.799) were considered R, while all of the patients harboring high Akk levels (Akk^{H}, Akk≥4.8) and no Akk (Akk0) at all were considered NR. Altogether, we built a final score, named “TOPOSCORE”, to predict clinical benefit (OS at12 months) in cancer patients treated with ICI. C. Cox regression univariate analysis and Kaplan Meier curves of OS of the 245 patients with NSCLC according to the TOPOSCORE. D. Multivariate analyses (Cox proportional hazard model) of prognosis factors clinically relevant for OS12 months in advanced lung carcinoma in the discovery cohort, including SIG1+ and SIG2+ TOPOSCORE categories.

FIG. 5. Co-abundance Pearson networks and SIG1 and SIG2 associated with response or resistance to ICI in the validation cohort of NSCLC. The Venn diagram depicts the number of MGS species shared by discovery and validation networks and independently retrieved by each model.

FIG. 6. Performance of the TOPOSCORE in the validation cohort. A. As for FIG. 3 (discovery cohort), we applied the same calculation model and the same boundaries of the distribution of the FRnormCOUNT parameter in the validation cohort of 148 NSCLC patients by means of Kernel Density Estimation (KDE). SIG2 and SIG1 ratio distributions are depicted in oblique lines of distinct orientations and the boundaries between these two SIG are in the Y axis and individualize the same Grey Zone as for the discovery cohort. B. Cox regression univariate analysis and Kaplan Meier curves of OS of the 148 patients with NSCLC of the validation cohort according to the TOPOSCORE. C. Multivariate analyses (Cox proportional hazard model) of prognosis factors clinically relevant for OS12 months in advanced lung carcinoma, in the validation cohort, including SIG1+ and SIG2+ TOPOSCORE categories. D. Idem as in B, but taking into account all NSCLC cohorts (n=304) to analyze the effects of the TOPOSCORE survival prediction according to tumor PD-L1 expression (PD-L1<1, n=95 upper panel, PD-L1≥1, n=209, lower panel).

FIG. 7. Kaplan Meier survival curves for patients falling into SIG1+ or SIG2+ respectively according to lines of therapy and treatments. A-B. Advanced NSCLC patients from both cohorts (discovery+validation) were gathered to analyze the impact of the TOPOSCORE in various patient subsets (according to the line of ICI (A, left panel: 1 st line; A, right panel: 2 line and further) and ICI monotherapy (B)).

FIG. 8. MGS-versus PCR-based TOPOSCORE to predict overall survival. A. Idem as in FIGS. 4C and 4B but applying the TOPOSCORE to a prospective cohort of 61 NSCLC and 83 RCC patients. B. Comparison of performance between IMDC score and TOPOSCORE to segregate OS<12 and OS>12 in RCC patients. C. Cox regression analysis and Kaplan Meier survival curves in 393 NSCLC patients according to the TOPOSCORE falling within the SIG2 plus Akk^{L} region (SIG2+) (0<FRnormCOUNT <1.047) versus within SIG1 plus Akk^{0/H} region (SIG1+). D. Recalculation of the TOPOSCORE based on the restricted number of bacteria (n=24 instead of n=75) in 393 NSCLC patients. E. Similar analysis using PCR-based determination of the 24 bacteria-restricted TOPOSCORE on 313 patients with NSCLC (a subset of the whole 393 population, according to availability of fecal DNA).

FIG. 9. TOPOSCORE calculation in healthy volunteers and comparisons with cancer patients, whether R or NR to ICI. A. Beta-diversity (Principal Coordinate Analysis, PCoA) of fecal microbiota (microbial relative abundance) according to individual subgroups [NR NSCLC/RCC patient (OS<12 months), R NSCLC/RCC patient (OS>12 months), HV]. B. We implemented Partial Least Square Discriminant Analysis (PLS-DA) and the subsequent Variable Importance Plot (VIP) as a supervised analysis in order to identify the most discriminant microbial species among the three patient/HV groups. ANOSIM and PERMANOVA define the separation of the groups; p values define the significance of group separation after 999 permutations of the samples. Mann-Whitney U test p-values (*p<0.05,**p<0.01,***p<0.001) are indicated. C. The distribution of the FRnormCOUNT parameter, computed as in Materials and Methods section, is depicted in the two retrospective cohorts of NSCLC patients (n=393) and a third cohort of RCC patients (n=83) by means of Kernel Density Estimation (KDE) and in the HV (oblique lines). SIG2 and SIG1 ratio distributions for patients are depicted in oblique lines and blank respectively while the HV (n=5345) are in oblique lines of another orientation (mostly SIG2+, 7.8% featured in SIG1+).

FIG. 10. Functional gene pathways of the fecal microbiome according to the clinical outcome in NSCLC. A-B. Beta-diversity (Principal Coordinate Analysis, PCoA) of fecal microbiota microbial MetaCyc pathways according to patients' subgroups (ligght grey: non-responders, NR, OS<12 months), black: responders, R, OS≥12 months)) in discovery (A) and validation (B) NSCLC patients treated with ICI. C. Common metabolic pathways shared between SIG1 and/or SIG2 in the whole retrospective NSCLC cohort are shown in the Venn diagram. MetaCyc microbial pathways that are common among discovery/validation SIG1 and discovery/validation SIG2 are reported in Table 1. D-E. Partial Least Square Discriminant Analysis (PLS-DA) and the subsequent Variable Importance Plot (VIP) as a supervised analysis to identify the most discriminant stool MetaCyc microbial pathways for the discovery cohort (D) and the validation cohort (E) among the two patients' groups R versus NR. ANOSIM and PERMANOVA define the separation of the groups; p-values define the significance of group separation after 999 permutations of the samples. Fold ratios and Mann-Whitney U test p-values (*p<0.05,**p<0.01,***p<0.001) are indicated.

FIG. 11. Prevalence of MGS species belonging to SIG1 and SIG2. Prevalence of each MGS species belonging to SIG1 (black) and SIG2 (white), including the 23 species used in the PCR-based TOPOSCORE (grey arrow) in 393 NSCLC patients.

FIG. 12. Spearman correlations between microbial relative abundance using shotgun metagenomics-based sequencing or qRT-PCR. Spearman correlations indexes between the two methods of the 24 bacteria belonging to the restricted TOPOSCORE with their detection thresholds. Normalized values of the PCR gene products and relative abundances in MGS for each bacterium were correlated, the rho and p-values being annotated for each bacterium. Each dot represents one fecal sample DNA. Each graph depicts the result of one bacterium detection.

FIG. 13. Response to CAR T cells depending on the TOPOSCORE at baseline. Idem as in FIGS. 4C and 4B but applying the TOPOSCORE to a prospective cohort of 22 lymphoma patients prior to CAR-T CD19 infusion. Chi-square test for comparison of 6th months overall response rate and Cox regression univariate analysis and Kaplan Meier curves of progression-fre. survival (PFS) for the 22 NSCLC patients according to the 2 groups SIG1+ and SIG2+ calculated from MG of fecal samples at baseline.

FIG. 14. Co-abundance networks and Species Interacting Groups associated with overall survival in the discovery cohort of NSCLC.

The performance of the S score as predictor of the Overall Survival at 12 months (OS12) was analyzed by a Receiver Operating Characteristic (ROC) analysis. Two scores, 0.5351 and 0.7911, were identified as local maxima of the Youden index (indicated by red and green dots, respectively).

FIG. 15. Design and performance of the TOPOSCORE in the discovery cohort.

A. The distribution of the S score is depicted by means of Kernel Density Estimation (KDE). The boundaries between these two SIG distributions—identified as local maxima of the Youden index (0.5351 and 0.7911)—are indicated in the X axis and individualize the limits of the gray zone. The percentages of patients with OS<12 months is annotated in each SIG group. Patients' distribution was statistically significant as per X² statistics. Refer to Table 15 for details of p values. B. Cox regression univariate analysis and Kaplan Meier curves of overall survival (OS) for the 245 NSCLC patients according to the 3 regions within the S score: 1) a SIG1 region (0<x<0.535); 2) a Gray zone (0.535≤x<0.791); 3) a SIG2 region (x≥0.791) calculated from MG of fecal samples at baseline. C. Sankey diagram for the longitudinal follow up of patient categorization using the S score in 32 NSCLC patients. D. Decision-making tree to calculate the TOPOSCORE. Step 1 consists in calculating the S score (number of SIG2 MGS present in individual patient stool divided by 45 (frequency (f) SIG2) minus number of SIG1 MGS present in individual patient stool divided by 37 (frequency (f) SIG1))+1 divided by 2. If the S score falls into the Gray zone (0.535≤x<0.791), the Akkermansia muciniphila relative abundance will allow to further classify the patient stool as follows: all patients harboring physiological “normal” A. muciniphila (Akk) relative abundances (0<Akk≤4.799) should be considered “OS>12”, while all the other “Gray zone” patients (harboring high Akk levels (Akk^{High}, Akk≥4.8) and no Akk (Akk0)) have to be considered OS<12, allowing a final binary categorization into “SIG1+” and “SIG2+” respectively. TOPOSCORE values are indicated in circles. E. Cox regression univariate analysis and Kaplan Meier curves of progression free- and overall survival (left and right panels) in the 245 NSCLC patients according to the binary categorization of the TOPOSCORE. Refer to multivariate analyses in Table 2A.

FIG. 16. Performance of the TOPOSCORE in the validation cohort of NSCLC patients.

A. As for FIG. 14 (discovery cohort), we applied the same calculation model and the same boundaries of the S score distribution in the validation cohort of 254 NSCLC patients by means of Kernel Density Estimation (KDE) and annotated the % of patients with OS<12 in each group. Refer to Table 15 for statistical analyses. B. Cox regression univariate analysis and Kaplan Meier curves of PFS and OS in the 254 NSCLC patient validation cohort according to the TOPOSCORE. Refer to multivariate analyses in Table 2B. C Sankey diagram for the longitudinal follow up of patient categorization using the TOPOSCORE in 67 NSCLC patients. D-F. Idem as in B but taking into account both NSCLC cohorts to analyze the effects of the TOPOSCORE on OS according to tumor PD-L1 expression (D) or focusing on first line immunotherapy (E) or first line chemo-immunotherapy (F).

FIG. 17. Validation of the TOPOSCORE in urinary tract malignancies and friendly-user qPCR-based TOPOSCORE.

A. Bar graph recapitulating the proportion of individuals (HV or cancer patients) diagnosed with a gut dysbiosis (defined as “SIG1+” using TOPOSCORE) according to histotype, and treatment line (also refer to FIG. 18B). B. Application of the TOPOSCORE in a prospective cohort of 133 UC and 83 RCC patients to predict PFS and OS in Cox regression analyses and Kapalnmeier curves of survival. Refer to Table 15. C. Cox regression analysis and Kaplan Meier survival curves in 393 NSCLC patients according to the 83 (left panel) versus 21 (right panel) MGS-based TOPOSCORE based on shotgun MG data. D. Prototypic examples of two Spearman correlations between qPCR and shotgun MG-based calculation of bacterium relative abundances (also refer to FIG. 21). E-F. Similar analysis as FIG. 17C right panel using qPCR-based determination of bacterial abundance using the 21 bacteria-restricted TOPOSCORE in a first cohort of 286 NSCLC patients (a subset of the whole 393 population, according to availability of fecal DNA) (E) and in a subsequent cohort of 96 patients (F).

FIG. 18. TOPOSCORE calculation in healthy volunteers and comparisons with cancer patients classified according to OS12.

A. We implemented Partial Least Square Discriminant Analysis (PLS-DA) and the subsequent Variable Importance Plot (VIP) as a supervised analysis in order to identify the most discriminant microbial species among the patient (OS< or >12) and HV groups. ANOSIM and PERMANOVA define the separation of the groups; p values define the significance of group separation after 999 permutations of the samples. Mann-Whitney U test p-values (*p<0.05, **p<0.01,***p<0.001) are indicated. B. SIG1 and SIG2 ratio distributions for all NSCLC patients and HV are depicted.

FIG. 19. Machine learning algorithms based on RF or HQ-MAGs and SIG-related functional pathways.

A-B. Machine learning algorithms using Random Forest (RF) classifier trained on the discovery cohort. Siamcat algorithm (A) and abundances of 284 high-quality Metagenome-Assembled Genomes (HQ-MAGs-based) (B) were used for the RF model, and classifier performance was measured by ROC curves and AUC value. C-E. Microbial pathways analysis. Enumeration of the metabolic MetaCyc pathways distinct or shared between SIG1 and/or SIG2 pools of bacteria in the whole NSCLC cohort of 499 NSCLC patients are shown in the Venn diagram and Table S3 (C). Beta-diversity (D). Partial Least Square ordination plot of fecal microbiota MetaCyc pathways in the whole cohort of 499 NSCLC patients treated with ICI and categorized with TOPOSCORE (Black: SIG1+, green: SIG2+). ANOSIM metric defines the separation of the groups; p-value defines the significance of group separation after 999 permutations of the samples (D). Partial Least Square Discriminant Analysis (PLS-DA) and the subsequent Variable Importance Plot (VIP) as a supervised analysis to identify the most discriminant stool MetaCyc microbial pathways for SIG1+ and SIG2+ patients. Mann-Whitney U test p-values (*p<0.05, **p<0.01, ***p<0.001) are indicated (E).

FIG. 20. Prevalence of MGS species belonging to SIG1 and SIG2.

Prevalence of each MGS species belonging to SIG1 and SIG2, including the 21 species used in the aPCR-TOPOSCORE (gray arrows) in 393 NSCLC patients.

FIG. 21. Spearman correlations for microbial abundance between shotgun metagenomics-based sequencing and qPCR.

Spearman correlations indexes between the two detection methods of the 19 bacteria (the two others being presented in FIG. 17) belonging to the restricted TOPOSCORE with their detection thresholds. Normalized values of the qPCR quantification and relative abundances in MGS for each bacterium were correlated, the rho and p-values being annotated for each bacterium. Each dot represents one fecal sample DNA. Each graph depicts the result of one bacterium detection.

FIG. 22. TOPOSCORE in colorectal cancer predicts overall survival only in patients treated by immunotherapy (anti-PDL-1 Ab).

Cox regression analysis and Kaplan Meier survival curves according to the 21 MGS-based PCR assai in 150 colorectal cancer randomised between two arms with or without anti-PD-L1 Ab (only Microsatellite sufficiency (MSS) shown) In ATEZOTRIBE clinical trial.

FIG. 23. AUC calculated with 10 combinations of 50 bacterial species

Receiver Operating Characteristic (ROC) curves of the best ten Random Forest (RF) models classifier trained on the unified MetaPhlAn4 database of discovery and validation cohorts (n=499 patients) to predict clinical benefit to ICI. Using the best 50 MGS selected with RF, their optimized combinations gave over 1˜possible combinations, then ten best models were developed, exhibiting a high degree of similarity in terms of Area Under Curve (AUC), Specificity (Sp) and Sensitivity (Se).

### DETAILED DESCRIPTION OF THE PREFERRED EMBODIMENTS

According to a first aspect, the present invention pertains to a method of diagnosing intestinal dysbiosis in an individual, comprising:
(i) in a sample from said individual comprising intestinal microbiota, assessing the presence or absence of bacterial species of a first species interacting group (“SIG1”) consisting of N₁ bacterial species comprising at least 5, preferably at least 6, more preferably at least 7 bacterial species selected from the group consisting of Dialister invisus, Enterococcus faecalis, Haemophilus parainfluenzae, Veillonella atypica, Eggerthella lenta, Erysipelatoclostridium ramosum, Enterocloster bolteae, Alloscardovia omnicolens, Bifidobacterium dentium, Campylobacter concisus, Clostridium perfringens, Enterococcus durans, Enterococcus faecium, Klebsiella pneumoniae, Lacticaseibacillus paracasei, Lacticaseibacillus rhamnosus, Lactobacillus delbrueckii, Lactobacillus gasseri, Lactobacillus vaginalis, Lactococcus lactis, Lactococcus laudensis, Ligilactobacillus salivarius, Limosilactobacillus fermentum, Limosilactobacillus oris, Megasphaera micronuciformis, Mogibacterium diversum, Scardovia wiggsiae, Streptococcus anginosus, Streptococcus gordonii, Streptococcus infantis, Streptococcus mutans, Streptococcus parasanguinis, Streptococcus salivarius, Veillonella dispar, Veillonella parvula, Veillonella rogosae, Enterocloster aldensis, Enterocloster asparagiformis, Faecalimonas umbilicata, Gordonibacter urolithinfaciens, Actinomyces graevenitzii, Anaerostipes caccae, Blautia producta, Campylobacter gracilis, Clostridium innocuum, Clostridium scindens, Clostridium symbiosum, Collinsella SGB14754, Enorma massiliensis, Enterocloster clostridioformis, Fournierella massiliensis, Granulicatella adiacens, Hungatella hathewayi, Proteus mirabilis and Streptococcus oralis;
(ii) in a sample from said individual comprising intestinal microbiota, assessing the presence or absence of bacterial species of a second species interacting group (“SIG2”) consisting of N₂ bacterial species comprising at least 5, 6 or 7, preferably at least 10 to 12, more preferably at least 14 bacterial species selected from the group consisting of Dorea formicigenerans, Dorea longicatena, Eubacterium ventriosum, Eubacterium rectale, Holdemania filiformis, Parasutterella excrementihominis, Anaerostipes hadrus, Blautia obeum, Blautia wexlerae, Faecalibacterium prausnitzii, Roseburia hominis, Roseburia intestinalis, Roseburia inulinivorans, Ruminococcus bicirculans, Ruminococcus lactaris, Candidatus Cibiobacter qucibialis, Clostridiales bacterium KLE1615, Faecalibacillus intestinalis, Lachnospira eligens, Lacrimispora celerecrescens, Adlercreutzia equolifaciens, Agathobaculum butyriciproducens, Anaerobutyricum hallii, Blautia faecis, Blautia massiliensis, Clostridia unclassified SGB4447, Clostridiaceae bacterium, Clostridium sp AF34 10BH, Clostridium sp AF36 4, Eubacteriaceae bacterium, Fusicatenibacter saccharivorans, Lachnospira pectinoschiza, Lachnospiraceae bacterium, Roseburia faecis, Anaerotignum faecicola, Clostridiaceae bacterium OM08 6BH, Clostridiaceae unclassified SGB4769, Clostridiales unclassified SGB15145, Clostridium fessum, Clostridium sp AM22 11AC, Clostridium sp AM33 3, Clostridium sp AM49 4BH, Coprobacter fastidiosus, Coprococcus comes, Coprococcus eutactus, Eubacterium ramulus, Faecalibacterium SGB15346, Firmicutes bacterium AF16 15, Gemmiger formicilis, Lachnospira sp NSJ 43, Lachnospiraceae bacterium OM04 12BH, Lachnospiraceae bacterium WCA3 601 WT 6H, Lacrimispora amygdalina, Mediterraneibacter butyricigenes, Oscillibacter sp ER4, Phocaeicola massiliensis and Roseburia sp AF02 12;
(iii) calculating a FRNormCount as follows:

$$FRNormCount = \frac{\frac{{N}_{SIG 1}}{N 1}}{\frac{{N}_{SIG 2}}{N 2}} ,$$

wherein N_{SIG1} is the number of bacterial species of SIG1 present in the sample and N_{SG2} is the number of bacterial species of SIG2 present in the sample; and/or
(iv) calculating a S score as follows:

$$S = ( \frac{{N}_{SIG 2}}{N 2} - \frac{{N}_{SIG 1}}{N 1} - 1 ) / 2 ,$$

wherein N_{SG1} is the number of bacterial species of SIG1 present in the sample and N_{SG2} is the number of bacterial species of SIG2 present in the sample;
wherein if the FRNormCount is inferior to a predetermined threshold TOPO₁ and/or if the S score is superior to a predetermined threshold S₂, 1 is assigned to the TOPOSCORE and the individual is likely not to have intestinal dysbiosis, and if the FRNormCount is superior to a predetermined threshold TOPO₂ superior to TOPO₁ and/or if the S score is inferior to a predetermined threshold S₁ inferior to S₂, 5 is assigned to the TOPOSCORE and the individual is likely to have intestinal dysbiosis.

When performing the above method, any appropriate technique known by the skilled person can be used to assess the presence of each bacterial species, such as metagenomic sequencing (MGS) and other sequencing-based techniques, PCR and other amplification-based techniques, hybridization (for example using a nucleic microarray) and any other appropriate method known to the person of skills in the art. The skilled person can identify, by routine work, nucleic acid sequences specific for a given microorganism, that can be chosen to specifically detect said microorganism.

The above method can be performed by measuring the presence of the recited SIG1 and SIG2 bacteria in any sample from the individual which reflects his/her intestinal microbiota. Examples of such samples include fecal material (feces), ieal material (such as a biopsy of ileum mucosae, ileal fresh mucoase-associated bacterial biofilm biopsy or ileal mucus), colonic material and any gut mucosal material. The skilled person knows how to collect and store the samples in conditions enabling survival of the bacterial species, and is free to choose appropriate techniques for preparing the microbial composition, which can be freshly-prepared liquid, reconstituted from freeze-dried material or any other conditioning enabling the analysis of the individual'≤gut microbiota. The presence or absence of SIG1 and SIG2 bacterial species can also be assessed from a plasma sample, from which DNA (cell-free DNA from the individual and microbes DNA) is extracted and sequenced to assess the presence of bacterial species (Micronoma™ technology). Of course, the same sample is preferably used to assess the presence of bacteria of SIG1 and SIG2.

According to a first specific embodiment of the above method, (i) SIG1 consists of N₁ bacterial species selected from the group consisting of Dialister invisus, Enterococcus faecalis, Haemophilus parainfluenzae, Veillonella atypica, Eggerthella lenta, Erysipelatoclostridium ramosum, Enteroclosterbolteae, Alloscardovia omnicolens, Bifidobacterium dentium, Campylobacter concisus, Clostridium perfringens, Enterococcus durans, Enterococcus faecium, Klebsiella pneumoniae, Lacticaseibacillus paracasei, Lacticaseibacillus rhamnosus, Lactobacillus delbrueckii, Lactobacillus gasseri, Lactobacillus vaginalis, Lactococcus lactis, Lactococcus laudensis, Ligilactobacillus salivarius, Limosilactobacillus fermentum, Limosilactobacillus oris, Megasphaera micronuciformis, Mogibacterium diversum, Scardovia wiggsiae, Streptococcus anginosus, Streptococcus gordonii, Streptococcus infantis, Streptococcus mutans, Streptococcus parasanguinis, Streptococcus salivarius, Veillonella dispar, Veillonella parvula, Veillonella rogosae, Enterocloster aldensis, Enterocloster asparagiformis, Faecalimonas umbilicata and Gordonibacter urolithinfaciens and (ii) SIG2 consists of N₂ bacterial species selected from the group consisting of Dorea formicigenerans, Dorea longicatena, Eubacterium ventriosum, Eubacterium rectale, Holdemania filiformis, Parasutterella excrementihominis, Anaerostipes hadrus, Blautia obeum, Blautia wexlerae, Faecalibacterium prausnitzii, Roseburia hominis, Roseburia intestinalis, Roseburia inulinivorans, Ruminococcus bicirculans, Ruminococcus lactaris, Candidatus Cibiobacter qucibialis, Clostridiales bacterium KLE1615, Faecalibacillus intestinalis, Lachnospira eligens, Lacrimispora celerecrescens, Adlercreutzia equolifaciens, Agathobaculum butyriciproducens, Anaerobutyricum hallii, Blautia faecis, Blautia massiliensis, Clostridia unclassified SGB4447, Clostridiaceae bacterium, Clostridium sp AF34 10BH, Clostridium sp AF36 4, Eubacteriaceae bacterium, Fusicatenibacter saccharivorans, Lachnospira pectinoschiza, Lachnospiraceae bacterium and Roseburia faecis.

According to a second specific embodiment of the above method, (i) SIG1 consists of N₁ bacterial species selected from the group consisting of Veillonella atypica, Erysipelatoclostridium ramosum, Enterocloster bolteae, Enterocloster aldensis, Alloscardovia omnicolens, Bifidobacterium dentium, Campylobacter concisus, Clostridium perfringens, Lacticaseibacillus paracasei, Lactobacillus gasseri, Lactobacillus vaginalis, Ligilactobacillus salivarius, Limosilactobacillus fermentum, Limosilactobacillus oris, Megasphaera micronuciformis, Streptococcus anginosus, Streptococcus gordonii, Streptococcus mutans, Streptococcus parasanguinis, Streptococcus salivarius, Veillonella dispar, Veillonella parvula, Actinomyces graevenitzii, Anaerostipes caccae, Blautia producta, Campylobacter gracilis, Clostridium innocuum, Clostridium scindens, Clostridium symbiosum, Collinsella SGB14754, Enorma massiliensis, Enterocloster clostridioformis, Fournierella massiliensis, Granulicatella adiacens, Hungatella hathewayi, Proteus mirabilis and Streptococcus oralis, and (ii) SIG2 consists of N₂ bacterial species selected from the group consisting of Dorea formicigenerans, Dorea longicatena, Eubacterium ventriosum, Eubacterium rectale, Anaerostipes hadrus, Blautia wexlerae, Faecalibacterium prausnitzii, Roseburia hominis, Roseburia intestinalis, Roseburia inulinivorans, Ruminococcus bicirculans, Ruminococcus lactaris, Candidatus Cibiobacter qucibialis, Clostridiales bacterium KLE1615, Faecalibacillus intestinalis, Lachnospira eligens, Agathobaculum butyriciproducens, Anaerobutyricum hallii, Blautia massiliensis, Clostridiaceae bacterium, Clostridium sp AF34 10BH, Lachnospira pectinoschiza, Anaerotignum faecicola, Clostridiaceae bacterium OM08 6BH, Clostridiaceae unclassified SGB4769, Clostridiales unclassified SGB15145, Clostridium fessum, Clostridium sp AM22 11AC, Clostridium sp AM33 3, Clostridium sp AM49 4BH, Coprobacter fastidiosus, Coprococcus comes, Coprococcus eutactus, Eubacterium ramulus, Faecalibacterium SGB15346, Firmicutes bacterium AF16 15, Gemmiger formicilis, Lachnospira sp NSJ 43, Lachnospiraceae bacterium OM04 12BH, Lachnospiraceae bacterium WCA3 601 WT 6H, Lacrimispora amygdalina, Mediterraneibacter butyricigenes, Oscillibacter sp ER4, Phocaeicola massiliensis and Roseburia sp AF02 12.

According to a third specific embodiment of the above method, (i) SIG1 consists of N₁ bacterial species selected from the group consisting of Veillonella atypica, Erysipelatoclostridium ramosum, Enterocloster bolteae, Enterocloster aldensis, Alloscardovia omnicolens, Bifidobacterium dentium, Campylobacter concisus, Clostridium perfringens, Lacticaseibacillus paracasei, Lactobacillus gasseri, Lactobacillus vaginalis, Ligilactobacillus salivarius, Limosilactobacillus fermentum, Limosilactobacillus oris, Megasphaera micronuciformis, Streptococcus anginosus, Streptococcus gordonii, Streptococcus mutans, Streptococcus parasanguinis, Streptococcus salivarius, Veillonella disparand Veillonella parvula and (ii) SIG2 consists of N₂ bacterial species selected from the group consisting of Dorea formicigenerans, Dorea longicatena, Eubacterium ventriosum, Eubacterium rectale, Anaerostipes hadrus, Blautia wexlerae, Faecalibacterium prausnitzii, Roseburia hominis, Roseburia intestinalis, Roseburia inulinivorans, Ruminococcus bicirculans, Ruminococcus lactaris, Candidatus Cibiobacter qucibialis, Clostridiales bacterium KLE1615, Faecalibacillus intestinalis, Lachnospira eligens, Agathobaculum butyriciproducens, Anaerobutyricum hallii, Blautia massiliensis, Clostridiaceae bacterium, Clostridium sp AF34 10BH and Lachnospira pectinoschiza.

According to specific embodiments of the above method, the number N₁ of bacterial species in the interacting group of bad prognosis (“SIG1”) is equal to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 6, 37, 38, 39, 40, 41, 42, 43, 44, 45, 46, 47, 48, 49, 50, 51, 52, 53, 54, 55 or more.

According to specific embodiments, the bacterial species included in SIG1 are all comprised in any one of the the lists indicated above in points (i).

According to other specific embodiments, the bacterial species included in SIG1 comprise at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 6, 37, 38, 39 or 40 bacterial species recited in the list indicated above in point (i), as well as other bacterial species also of dismal prognosis.

According to other specific embodiments of the above method, the number N₂ of bacterial species in the interacting group of favorable prognosis (“SIG2”) is equal to 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 51, 52, 53, 54, 55, 56, 57 or more.

According to specific embodiments, the bacterial species included in SIG2 are all comprised in any one of the lists indicated above in points (ii).

According to other specific embodiments, the bacterial species included in SIG2 comprise a least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 bacterial species recited in the list indicated above in point (ii), as well as other bacterial species also of good prognosis.

According to a particular embodiment, the total number of SIG1 and SIG2 bacteria (N₁+N₂) is at least 15, 16, 17 or 18, preferably at least 19, 20, 21 or 22, more preferably at least 23, 24 or 25.

According to a particular embodiment, the presence of a higher number of beneficial bacteria (SIG2) is assessed, compared to the number of harmfull bacteria (SIG1), athough a test with N₂/N₁=1 was also shown to provide valuable results—see Example 7 below. For example, about % to %, e.g. about 2/3 of the bacteria are beneficial bacteria (SIG2) and % to %, e.g. about ⅓ are harmfull (SIG1). N₂/N₁ is preferably in the interval [1, 4], more preferably in the interval [2, 3]. Noticeably, the relevance of the test will be higher if the tested bacteria belong to distinct genera.

In Example 2 below, N₁=40 (all the bacterial species recited in (i) of the first specific embodiment) and N₂=34 (all the bacterial species recited in (ii) of the first specific embodiment). Under these conditions and based on the results obtained on the studied cohorts, TOPO₁=0.37 and TOPO₂=1.047. The skilled person can refine these thresholds, for example by reproducing the experiments disclosed below on a bigger cohort of patients, or adapt it to specific situations (e.g., particular subgroups of patients, based on their geographical origin, clinical status—type or grade of cancer, genetic peculiarities, etc.). An important aspect of the claimed method is however that it applies to all cancer patients, as well as to healthy individuals (for example, donors of fecal material can be tested to assess whether they are good donors for Fecal Microbial Transplantation (FMT) to cancer patients).

In Example 9 below, N₁=37 (all the bacterial species recited in (i) of the second specific embodiment) and N₂=45 (all the bacterial species recited in (ii) of the second specific embodiment). Under these conditions and based on the results obtained on the studied cohorts, S₁=0.535 and S₂=0.791. The skilled person can also refine these thresholds, as mentioned above regarding TOPO₁ and TOPO₂.

Of course, the skilled person can also easily recalculate the values of TOPO₁ and TOPO₂ and/or S₁ and S₂ to adapt these to any combination of species interacting groups (SIG1 and SIG2 defined above), by reproducing the experiments described below with data corresponding to the bacterial species comprised in said SIGs.

The above method thus provides an information on the probability that the tested individual has intestinal dysbiosis, since:
when FRNormCount<TOPO₁ and/or when S>S₂, the individual is likely not to have intestinal dysbiosis, and
when FRNormCount>TOPO₂ and/or when S<S₁, the individual is likely to have intestinal dysbiosis.

To resolve the grey zone (i.e., when TOPO₁≤FRNormCount≤TOPO₂ and/or when S₁≤S≤S₂), the trichotomized approach described in WO 2022/157207, based on Akkermansia species (Akk) relative abundance, can advantageously be used. Indeed, the inventors demonstrated that patients falling within the grey zone and harboring low Akk relative abundance (e.g., 0<Akk≤4.799) can be considered as responders, while patients in the grey one harboring either high Akk relative abundance (Akk≥4.8) or no Akk (Akk=0) can be considered as non-responders (see Example 2 in the experimental part below). The content of WO 2022/157207 is incorporated herein by reference. The “relative abundance” of a definite bacterial species is defined as the fraction of the entire bacterial ecosystem belonging to this bacterial species. The relative abundance can be expressed as a percentage or within the closed interval [0:1], where 1 stands for the maximum fraction available for a single bacterial species (i.e., a bacterial species with a relative abundance equal to 1 means that 100% of the bacteria present in the sample are of the considered species).

Hence, based on the FRNormCount and/or the S score defined above and on the relative abundance of bacteria of the Akkermansia genus (e.g., Akkermansia muciniphila and/or Akkermansia SGB9228 and/or other Akkermansia species), a score can be assigned to the individual to reflect the probability that he/she has an intestinal dysbiosis. This score, defined herein as the “TOPOSCORE”, is a number between 1 and 5, with a risk of intestinal dysbiosis which increases with the value of the TOPOSCORE.

The present invention thus also pertains to a method as above described, wherein if TOPO₁, FRNormCount≤TOPO₂ and/or S₁≤S≤S₂ (“grey zone”), the relative abundance of bacteria of the Akkermansia genus (Akk) is measured in a fecal material sample from said individual, wherein:
a) if bacteria of the Akkermansia genus are present in the sample below a predetermined threshold (“Akk superior threshold”), 2 is assigned to the TOPOSCORE and the patient is likely not to have intestinal dysbiosis; and
b) if no Akkermansia is present in the sample, 3 is assigned to the TOPOSCORE and the individual is likely to have intestinal dysbiosis;
c) if bacteria of the Akkermansia genus are present in the sample above the Akk superior threshold, 4 is assigned to the TOPOSCORE and the individual is likely to have intestinal dysbiosis.

An example of threshold that can be used as “Akk superior threshold” in the frame of the invention is disclosed in WO 2022/157207 and in the experimental part below. Typically, the 75^{th} or 77^{th} percentile of the relative abundance of bacteria of the Akkermansia genus can be chosen as Akk superior threshold. In the cohort described in WO 2022/157207, this led to a value of 4.799%; based on these results, a value of 4.8% was successfully used in the experimental part below. Of course, the skilled artisan can adapt or refine this threshold, depending on the technique used to measure the relative abundance of Akkermansia muciniphila and/or Akkermansia massiliensis (formerly called Akkermansia SGB9228 in WO 2022/157207) and/or of the Akkermansia genus (for example, metagenomics, quantitative PCR, hybridization on a microarray or pyrosequencing), the species of Akkermansia which is(are) detected, the specific condition of the patient, the patient'≤food habits, the specific ICI used for the treatment and other possible factors. For example, the threshold to be considered when performing the above method can be predetermined by measuring the relative abundance of Akkermansia muciniphila and/or Akkermansia massiliensis, and/or of the Akkermansia genus in a representative cohort of individuals having the same cancer as the patient for whom a prognostic is sought, and choosing as threshold the value of the 75^{th} percentile. This threshold can be slightly different for Akkermansia muciniphila and for Akkermansia massiliensis.

WO 2022/157207 discloses several methods for assessing the relative abundance of Akkermansia, which can also be used when performing the present invention. In particular, this can be done by quantitative PCR using, for example, one of the primer pairs disclosed in the table page 22 of WO 2022/157207 A1, for example the primers AkkermansiaSGB9226/9228_F and AkkermansiaSGB9226/9228_R which hybridize to both Akkermansia muciniphila and Akkermansia massiliensis genomes.

Of course, the step of assessing the relative abundance of Akkermansia can be performed using any sample from the individual which reflects his/her intestinal microbiota, as above-described.

As illustrated in Example 6 below, the inventors showed that a clinically relevant TOPOSCORE could be obtained with a subset of 7 bacterial species selected from the 40 bacterial species disclosed above as belonging to SIG1 (first specific embodiment), and a subset of 16 bacterial species selected from the 34 bacterial species disclosed above as belonging to SIG2 (first specific embodiment). Noticeably, the TOPOSCORE so obtained was relevant even without recalculating the TOPO₁ and TOPO₂ thresholds. The inventors' hypothesis is that the presence of SIG1 bacteria is more discriminant than that of SIG2 bacteria, which have a higher prevalence (as shown in FIG. 11). The number of SIG2 bacterial species to be detected in the method could thus be further decreased (as shown in Example 7) without loosing accuracy.

According to a particular embodiment of the method described above, the first species interacting group (SIG1) comprises at least 5, preferably at least 6 and more preferably all of the SIG1 bacteria detected by the method illustrated in Example 6 below, i.e., Dialister invisus, Enterococcus faecalis, Haemophilus parainfluenzae, Veillonella atypica, Eggerthella lenta, Erysipelatoclostridium ramosum and Enterocloster bolteae.

According to another particular embodiment of the method described above, the second species interacting group (SIG2) comprises at least 5, preferably at least 6, 7, 8, 9, 10, 11, 12, 13, 14, 15 or all of the SIG2 bacteria detected by the method illustrated in Example 6 below, i.e., Dorea formicigenerans, Dorea longicatena, Eubacterium ventriosum, Eubacterium eligens, Eubacterium rectale, Holdemania filiformis, Parasutterella excrementihominis, Anaerostipes hadrus, Blautia obeum, Blautia wexlerae, Faecalibacterium prausnitzii, Roseburia hominis, Roseburia intestinalis, Roseburia inulinivorans, Ruminococcus bicirculans and Ruminococcus lactaris.

In Example 13 below, the inventors showed that a clinically relevant TOPOSCORE could be obtained with a subset of 5 bacterial species selected from the 37 bacterial species disclosed above as belonging to SIG1 (second specific embodiment), and a subset of 15 bacterial species selected from the 45 bacterial species disclosed above as belonging to SIG2 (second specific embodiment). Noticeably, the TOPOSCORE so obtained was relevant even without recalculating the S₁ and S₂ thresholds.

According to a particular embodiment of the method described above, the first species interacting group (SIG1) comprises at least 3, preferably at least 4 and more preferably all of the SIG1 bacteria detected by the method illustrated in Example 13 below, i.e., Enterocloster bolteae, Erysipelatoclostridium ramosum, Veillonella atypica, Clostridium symbiosum and Hungatella hathewayi.

According to another particular embodiment of the method described above, the second species interacting group (SIG2) comprises at least 5, preferably at least 6, 7, 8, 9, 10, 11, 12, 13, 14 or all of the SIG2 bacteria detected by the method illustrated in Example 13 below, i.e., Anaerostipes hadrus, Blautia wexlerae, Dorea formicigenerans, Dorea longicatena, Eubacterium rectale, Eubacterium ventriosum, Faecalibacterium prausnitzii, Roseburia hominis, Roseburia intestinalis, Roseburia inulinivorans, Ruminococcus bicirculans, Ruminococcus lactaris, Coprococcus comes, Gemmiger formicilis and Phocaeicola massiliensis.

According to another embodiment, the method according to the invention further comprises
(i) in a sample from said individual comprising intestinal microbiota, assessing the presence or absence of functional pathways specifically related to SIG1 bacteria in the metagenome, wherein at least part of said SIG1-specific pathways are selected from purine nucleobase and pyrimidine deoxynucleotide phosphorylation and degradation, guanosine nucleotide de novo biosynthesis and L histidine degradation,
(ii) in a sample from said individual comprising intestinal microbiota, assessing the presence or absence of functional pathways specifically related to SIG2 bacteria in the metagenome, wherein at least part of said SIG2-specific pathways are selected from autophagy-related pathways (polyamines such as S-adenosyl-L-methionine salvage, L-ornithine, L-arginine biosynthesis, putrescine biosynthesis) and sulfur oxidation, superpathway of β-D-glucuronide and D-glucuronate degradation, superpathway of L-alanine and L-aspartate, L-asparagine biosynthesis,
wherein the presence of SIG2-specific functional pathways in the metagenome in the absence of SIG1-specific functional pathways indicates that the individual is of a “SIG2” genotype, and the presence of SIG1-specific functional pathways in the metagenome in the absence of SIG2-specific functional pathways indicates that the person is of a “SIG1” genotype.

In the above embodiment, the lists of SIG-1 or SIG2-specific pathways are not exhaustive, and further such specific pathways can also be detected.

Functional pathway determination can be performed using shotgun-based metagenomics sequencing of the stools. To explore putative microbial functions underlying SIG1 and SIG2 compositions, one can employ an analysis of metagenomic pathways by means of HUMAnN 3.0 pipeline (or more updated versions) or other pipelines employing MetaCyc-related pathway reconstruction. All these pipelines first annotates microbial-specific gene hits according to the Kyoto Encyclopedia of Genes and Genomes Orthology, then reconstructs microbial metabolic pathways using the MetaCyc hierarchy.

According to a particular aspect of the invention, the method is performed for diagnosing intestinal dysbiosis in an individual who has a cancer, especially a cancer amenable to immune-oncology (I-O) therapy. The above method is especially useful for patients having a non small cell lung cancer (NSCLC) or a renal cell cancer (RCC) (e.g., a clear cell kidney cancer) or an urothelial cancer (UC) or a colorectal cancer or a lymphoma, especially patients having a cancer in stage IIIC/IV and/or receiving neoadjuvant I-O therapy in a context of operable tumor.

In the present text, the phrase “I-O therapy” includes immune checkpoint inhibitors (ICI), as well as CAR-T cells, adoptive TIL transfer and combinations thereof. In the context of the present invention, “I-O therapies” also include combined therapies including one of the above I-O agents and other antineoplastic treatments, such as chemotherapy, immunogenic chemotherapy (such as oxaliplatum-based or anthracycline) or radiotherapy, alone or especially any combination of an immune checkpoint inhibitor (ICI) with a tyrosine kinase inhibitor, taxanes, permetrexed, cis-platin and/or oxaliplatinum, or EGFR inhibitors, or cancver vaccines or antibody drug conjugates with immunogenic payload or CD3 based bispecific antibodies. For example, an immune checkpoint inhibitor (ICI) can advantageously be combined with a tyrosine kinase inhibitor in renal cancer or with platinum-based or taxane-based-chemotherapy in lung cancer.

In the context of the present invention, “ICI” include:
anti-PD1 antibodies (Ab), such as pembrolizumab (Keytruda), nivolumab (Opdivo), cemiplimab (Libtayo), toripalimab (Tuoyi), sintilimab (Tyvyt from InnoVent Biologics), tislelizumab (BeiGene), camrelizumab (AiRuiKa), penpulimab and zimberelimab; GSK anti-PD1 Ab (name to be checked in the website)
anti PDL-1 Ab, such as atezolizumab (Tecentriq), durvalumab (Imfinzi), avelumab (Bavencio), envafolimab (Enweida) and sugemalimab (Cejemly);
anti-CTLA4 Ab, such as ipilimumab (YervoY);
anti-Lag3 Ab, such as relatlimab;
bispecific antibodies targeting PD1 and Lag3, such as nivolumab/relatlimab (Opdualag);
bispecific antibodies targeting PD1 and CTLA-4, such as MED15752;
anti-Tim3 Ab;
anti-TIGIT Ab;
anti-OX40 Ab;
anti-41 BB Ab;
anti-VISTA Ab;
as well as other molecules exerting the same function(s), such as non-Ab molecules blocking any of the above immune checkpoints. According to a particular embodiment, the I-O therapy includes an anti-PD1/PDL-1 Ab, e.g. monoclonal Ab blocking PD1 or PDL-1 as those mentioned above.

According to another aspect, the invention pertains to a method of determining if a patient having a cancer amenable to immune-oncology (I-O) therapy is likely to be a good responder to said therapy, comprising assessing, using a method as described above, whether the patient has an intestinal dysbiosis, wherein a patient having an intestinal dysbiosis is less likely to respond to the I-O therapy than a non-dysbiotic patient. According to a particular embodiment of this theranostic method, the patient'≤TOPOSCORE is calculated, wherein the higher the patient'≤TOPOSCORE, the lower the probability that the patient responds to the I-O therapy in absence of a microbiota-centered intervention (MCI) before administration of said I-O therapy.

Noticeably, the TOPOSCORE is an individual score that helps clinicians in their decision relative to treatment strategy. For example, if the patient has a TOPOSCORE≤2, the patient is likely to respond to said I-O therapy, so that the medical team can envisage administering such a therapy to the patient. Conversely, if the patient has a TOPOSCORE>2, the patient is likely not to respond to said I-O therapy in absence of a microbiota-centered intervention (MCI) before administration of said I-O therapy. The medical team will then prefer to modulate the patient'≤microbiota to decrease his/her TOPOSCORE, preferably to a level of 1, before beginning the I-O therapy. Alternatively, the medical team can choose to start the I-O therapy at the same time or before a MCI, but with the knowledge that the individual is likely not to respond to it, so that a special attention is paid to the risk of resistance, with the idea to stop or modify the treatment rapidly if such resistance is confirmed. Indeed, in certain cases of treatment resistance, especially to ICI, it has been shown that ICI drugs can not only be useless, but even have deleterious effects, leading to rapid tumor progression (i.e., hyperprogressive disease or HPD). Identifying a patient likely to resist to an I-O treatment is thus of major importance to decide not to administer the I-O treatment to this patient, at least not without a compensatory treatment or combined therapy to avoid HPD onset.

In the present text, the phrases “microbiota-centered intervention (MCI)”, or “compensatory treatment”, designate any treatment having a direct or indirect effect on the intestinal microbiota composition, to lower the TOPOSCORE.

Examples of MCI that can be used in this context include:
fecal microbial transplantation (FMT), especially with fecal material from donor(s) with a TOPOSCORE of 1, preferably with FRNormCount=0, and
Akkermansia spp and/or Akkermansia muciniphila and/or Akkermansia massiliensis (especially when the TOPOSCORE is equal to 3, and not when it is equal to 4), possibly mixed with other beneficial bacteria,
oral vancomycin antibiotics (e.g., same protocol as for treating C. difficile infection),
phages killing bacteria of SIG1 (especially of the Enterocloster gen. nov. clade),
rare-cutting endonucleases such as Crispr Cas9 engineered to kill bacteria of SIG1 (especially of the Enteroclostergen. nov. clade),
retinoic acid
betablockers
any off target therapy restoring MAdCAM-1 (ileal or serum soluble)
camu-camu or castalagin-based prebiotics
mixtures of the above treatments.

The above theranostic method is particularly useful for determining if a patient having a cancer amenable to immune-oncology (I-O) therapy is likely to be a good responder to an immune checkpoint inhibitor(s) (ICI)-based therapy, and more particularly to a treatment with an anti-PD1 antibody, an anti-PD-L1 antibody, an anti-PD-L2 antibody and/or an anti-CTLA4 antibody such as those described above, alone or combined with another antineoplastic agent.

According to a particular embodiment, the theranostic method according to the invention is used for determining if a patient having a cancer amenable to immune-oncology (I-O) therapy is likely to be a good responder to an anti-PD1 antibody or an anti-PD-L1 antibody.

The above theranostic method is also useful for determining if a patient having a cancer amenable to immune-oncology (I-O) therapy is likely to be a good responder to a treatment with a CAR T-cell targeting a tumor antigen (e.g., CD19).

According to another particular embodiment, the theranostic method according to the invention is used for determining if a patient having a renal cancer amenable to immune-oncology (I-O) therapy is likely to be a good responder to an anti-PD1 antibody or an anti-PD-L1 antibody combined with a tyrosine kinase inhibitor (TKI), such as, for example, axitinib (Inlyta), lenvatinib (Lenvima), cabozantinib (Cabometyx), sunitinib (Sutent), pazopanib (Votrient), sorafenib (Nexavar).

According to another particular embodiment, the theranostic method according to the invention is used for determining if a patient having a lung cancer amenable to immune-oncology (I-O) therapy is likely to be a good responder to an anti-PD1 antibody or an anti-PD-L1 antibody combined with platinum-based or taxane-based-chemotherapy.

According to another particular embodiment, the theranostic method according to the invention is used for determining if a patient having an urothelial cancer (UC) amenable to immune-oncology (I-O) therapy is likely to be a good responder to anti-PD1/PDL-1 and/or anti-CTLA4 Abs alone or combined together or combined with targeted therapies or chemotherapy.

According to another particular embodiment, the theranostic method according to the invention is used for determining if a patient having a colorectal cancer amenable to immune-oncology (I-O) therapy is likely to be a good responder to anti-PDL-1 based therapy (or anti-PD1Ab) alone or combined with FOLFIRI or FOLFIRINOX chemotherapy regimen and bevacizumab.

According to another particular embodiment, the theranostic method according to the invention is used for determining if a patient having a lymphoma amenable to immune-oncology (I-O) therapy is likely to be a good responder to CAR-T CD19.

According to another particular embodiment, the theranostic method according to the invention is used for determining if a patient having a cancer amenable to immunogenic chemotherapy (or antibody drug conjugate with a cytotoxic payload) is likely to be a good responder to an anti-PD-1/L1 antibody alone or combined to chemotherapy.

According to another particular embodiment, the theranostic method according to the invention is used for determining if a patient having a cancer amenable to immunogenic chemotherapy is likely to be a good responder to a CAR T-cell therapy.

According to another particular embodiment, the theranostic method according to the invention is used for determining if a patient having a cancer amenable to cancer vaccines is likely to be a good responder to the cancer vaccine alone or combined to an anti-PD-1/L1 antibody and/or combined with platinum-based or taxane-based-chemotherapy and/or combined with CAR T-cell therapy.

When performing the above methods, the TOPOSCORE is preferably calculated before beginning the I-O therapy (neoadjuvant or adjuvant setting), and optionally after at least partial tumor resection of the tumor.

The present invention also relates to the use of a TOPOSCORE calculated as described above, as a theranostics tool to determine if an individual needs an MCI, wherein when the TOPOSCORE≥3, the individual needs an MCI. Examples of appropriate MCI have been described above. According to a preferred embodiment, the MCI is to be performed by Fecal Microbial Transplantation (FMT) when the TOPOSCORE>3, and if the TOPOSCORE=3, the MCI is to be performed by Fecal Microbial Transplantation (FMT) and/or by administering a bacterial composition comprising bacteria of the Akkermansia genus. As already mentioned, FMT is preferably performed with material derived from fecal material from healthy donor(s) or cured patient(s) that bear a Toposcore=1, best with a FRNormCount=0 and/or a S Score=1. According to a preferred embodiment, the FMT is performed with fecal material from healthy donor(s) with FRNormCount=0.

According to another aspect of the present invention, a TOPOSCORE calculated as described above is used as a pharmacodynamics tool to monitor the evolution of the intestinal microbiota of an individual receiving a MCI and/or a treatment possibly impacting the intestinal microbiota and/or impacted by the intestinal microbiota. This is particularly useful, for example, to follow the capacity of FMT to restore eubiosis (TOPOSCORE=1) in a patient after FMT. This is also very interesting for monitoring the gut microbiota (precision medicine) in a patient receiving a treatment such as 1-0 therapy, chemotherapy, hormonotherapy, a tyrosine kinase inhibitor such as those mentioned above, especially since dysbiosis can result from such treatments and can cause resistance to these treatments.

According to the invention, a TOPOSCORE calculated as described above is also particularly useful to avoid administering an I-O therapy (alone or combined with other antineoplastic agents such as chemotherapy and TKI) to a patient likely to have a primary resistance thereto due to intestinal dysbiosis. Indeed, such treatments are very costly and, as already mentioned, they can have deleterious effects, so that it is preferable to identify potentially poor responders to avoid unnecessary side effects.

The TOPOSCORE calculated as described above is also particularly useful to stop or at least temporarily discontinue an 1-O therapy (alone or combined with other antineoplastic agents such as chemotherapy and TKI) if the patient develops a secondary resistance thereto due to intestinal dysbiosis.

Another aspect of the present invention is a method for assessing whether fecal material (originating from one donor or resulting from a mix of fecal materials from several donors) can be used in an MCI, using the TOPOSCORE as an indicator of the appropriateness of this fecal material. According to this aspect of the invention, a TOPOSCORE is calculated as described above from a sample of said fecal material, wherein if the TOPOSCORE is superior or equal to 3, the fecal material cannot be used in an MCI and if the TOPOSCORE is inferior or equal to 2, the fecal material can be used in an MCI. As already mentioned, fecal materials with FRNormCount=0 and S score=1 are preferred for use in an MCI.

The present invention also pertains to a kit of parts for performing the methods described above, which comprises means for detecting the presence of bacterial species of the SIG1 or SIG2, and also preferably means for assessing the relative abundance of Akkermansia. Such a kit can comprise, for example, a nucleic acid microarray (DNA chip) comprising probes specific for each of the bacterial species to be detected, and/or a primer pair specific for each of the recited bacterial species.

For example, the kit of parts of the invention comprises, for each bacterial species to be detected, a primer pair and/or a nucleic acid probe for specifically recognizing this bacterial species, wherein the bacterial species to be detected comprise at least 20, preferably at least 30, more preferably at least 40 of the following bacterial species, wherein the ratio N_{SIG1}:N_{SIG2} is about 1:3:
SIG1 bacteria: Streptococcus parasanguinis, Clostridium symbiosum, Streptococcus salivarius, Hungatella hathewayi, Clostridium scindens, Clostridium innocuum, Enteroclosteraldensis, Veillonella parvula, Enterocloster bolteae, Erysipelatoclostridium ramosum, Enterocloster clostridioformis, Bifidobacterium dentium, Veillonella dispar and Actinomyces graevenitzii;
SIG2 bacteria: Ruminococcus bicirculans, Faecalibacterium prausnitzii, Blautia wexlerae, Roseburia intestinalis, Gemmiger formicilis, Anaerostipes hadrus, Clostridiales bacterium KLE1615, Agathobaculum butyriciproducens, Dorea longicatena, Blautia massiliensis, Eubacterium rectale, Faecalibacterium SGB15346, Clostridium sp AF34 10BH, Lachnospira eligens, Lachnospiraceae bacterium WCA3 601 WT 6H, Clostridium fessum, Anaerobutyricum hallii, Candidatus Cibiobacter qucibialis, Anaerotignum faecicola, Clostridiaceae unclassified SGB4769, Roseburia hominis, Clostridiaceae bacterium, Oscillibacter sp ER4, Clostridiaceae bacterium OM08 6BH, Roseburia inulinivorans, Phocaeicola massiliensis, Lacrimispora amygdalina, Firmicutes bacterium AF16 15, Coprococcus eutactus, Eubacterium ventriosum, Clostridiales unclassified SGB15145, Faecalibacillus intestinalis, Coprococcus comes, Roseburia sp AF02 12, Clostridium sp AM49 4BH, Mediterraneibacter butyricigenes, Dorea formicigenerans, Coprobacter fastidiosus, Ruminococcus lactaris, Lachnospira sp NSJ 43, Clostridium sp AM22 11AC, Lachnospira pectinoschiza, Lachnospiraceae bacterium OM04 12BH, Clostridium sp AM33 3 and Eubacterium ramulus.

According to a particular embodiment, the kit of parts according to the invention also comprises means to assess the relative abundance of Akkermansia, such as, for example, a primer pair and/or probe specific of the Akkermansia genus. Examples of primer pairs which can be included in the kit are described in WO 2022/157207 A1.

According to another embodiment, the kit of parts according to the invention further comprises control primer and/or probe sets.

Other characteristics of the invention will also become apparent in the course of the description which follows of the biological assays which have been performed in the framework of the invention and which provide it with the required experimental support, without limiting its scope.

### EXAMPLES

Patient cohorts and specimen Feces-related translational research was conducted according to the ethical guidelines and approval of the local ethical committee (CCPPRB, Kremlin Bicétre). Feces for metagenomics analysis were performed under the study Oncobiotics (Discovery of Microbiome-based Biomarkers for Patients With Cancer Using Metagenomic Approach); Sponsors: Gustave Roussy, Cancer Campus, Grand Paris; Sponsor Protocol N: CSET 2017/2619, ID-RCB N: 2017-A02010-53). The written informed consent was obtained for all patients in accordance with the Declaration of Helsinki. General Data Protection Regulation procedures and anonymization rules have been applied according to Oncobiome H2020 model system already in place in the ClinicObiome, Gustave Roussy. All data and sample collection and all clinical trials are performed in compliance with regulatory, ethical, and European GDPR requirements. The bladder cancer cohort of patients allowing the IOPREDI/STRONG ancillary study (NCT03084471) biobanking and data mining was provided by AstraZeneca. IOPREDI (EudraCT Number: 2016-005068-33) is the French cohort of the STRONG phase IlIb trial (Sonpavde et al. 2022). Patients with bladder cancer who progressed on previous chemotherapy were treated with durvalumab (1500 mg every 4 weeks until progression). Baseline stool samples were used for MG analyses (n=133) and pooled with the kidney cancer cohort from ONCOBIOTICS.

Shotgun metagenomics sequencing and bioinformatic analysis For metagenomic analysis, the stools were processed for total DNA extraction and sequencing with Ion Proton technology following MetaGenoPolis (INRA) France, as previously reported (Carbonero et al. 2012; Dordevid et al. 2021). Metagenomic analysis of fastq files was performed following previously published guidelines (Routy et al. 2018) for taxonomic (MetaPhlAn 4.0) and functional (HUMAnN 3.0) profiling of metagenomes. These two pipelines leverage a set of 99,200 high-quality and fully annotated reference microbial genomes spanning 16,800 species and the 87.3 million UniRef90 functional annotations available in UniProt. The taxonomic profiling and quantification of organisms' relative abundances of all metagenomic samples were quantified using MetaPhlAn 4.0 with default parameters. In total, we identified 536 microbial species. Statistical analysis for FIG. 1. For the forthcoming statistical and metagenomic analysis, patients were randomly distributed into a discovery cohort (n=245) and validation cohort (n=254) by means of the Python “pandas” package v1.3.4, “df.sample” function. Overall survival (OS), defined as the time from immune checkpoint inhibitor treatment start until death from any cause, was estimated using the Kaplan-Meier method and compared with the log-rank test (Mantel-Cox method) in a univariate analysis. Multivariate survival analyses were performed using Cox regression models to determine hazard ratios and 95% confidence intervals for overall survival adjusting for variables with p-values <0.05 found in univariate analysis using the coxph function from the R survival package (v3.2-7). Survival analysis and Kaplan-Meier curves were computed (R v4.1.2 packages survival, survminer, Rcpp) for each microbial species harbored by each of the seven SIGs, retrieving survival curves parameters as per survfit function (records, n.max, n.start, events, rmean, se_rmean, median, 0.95LCL, 0.95UCL) and per survdiff function (Observed, Expected, (O-E)\hat{ }2/E, (O-E)\hat{ }2/V, Chi-squared, Pvalue) (Supplementary Table KM_calculations). These parameters were computed using the “high” and “low” relative abundances (considering the “high”≥0 and the “low”<0 of normalized and standardized relative abundance values as cut-off values). We then computed the difference (AHigh-Low mOS) and fold-ratio (log 2FRHigh/Low mOS) in median OS for each microbial species, finding out a hierarchy of positive to negative microbial species in terms of their contribution to patients' mOS.

### Statistical Analysis of Metagenomic Data

Within data matrices retrieved from the MetaPhlAn4 pipeline, only microbial species having a prevalence ≥2.5% were considered for subsequent analysis. For example 1 to 7, relative abundances of microbial species were first normalized then standardized using QuantileTransformer and StandardScaler methods from Sci-Kit learn package v1.0.1. Normalization using the output_distribution=‘normal’ option transforms each variable to a Gaussian-like distribution, ruling out the normalization with log 10-transformation coupled to pseudocount in order to avoid nonfinite values, while the standardization results in each normalized variable having a mean of zero and unit variance. For example 8 to 13, relative abundances of microbial species underwent transformation (multiplicative_replacement followed by centre-log-ratio clr functions, Sci-Kit learn package v1.0.1), then normalization and standardization using QuantileTransformer and StandardScaler methods from Sci-Kit learn package v1.0.1. Normalization using the output_distribution=‘normal’ option transforms the distribution of each variable to a Gaussian-like, while the standardization results in each normalized variable distribution having a mean of zero and unit varianceThese two steps of normalization and standardization ensure the proper comparison of variables with different dynamic ranges, such as microbial relative abundances. Centered log ratio transformation (CLR) was employed before doing SPRING, Spice-Easi, and SparCC network analysis. For microbiota analysis, measurements of a diversity (within sample diversity) such as Richness and Shannon index, were calculated at species level using the SciKit-bio package v0.5.6. Exploratory analysis of p-diversity (between sample diversity) was calculated using the ‘Bray-Curtis’ measure of dissimilarity and ‘complete linkage’ method, and represented in Principal Coordinate Analyses (PCoA) as an ordination plot. Metrics to compare groups of multivariate sample units (analysis of similarities—ANOSIM, permutational multivariate analysis of variance—PERMANOVA) were employed to assess significance in data points clustering. ANOSIM and PERMANOVA were automatically calculated after 999 permutations, as implemented in SciKit-bio package v0.5.6. We implemented Partial Least Square Discriminant Analysis (PLS-DA) and the subsequent Variable Importance Plot (VIP) as a supervised analysis wherein the VIP values (order of magnitude) are used to identify the most discriminant microbial species among the cohorts. A leave-one-out cross-validation (LOOCV) method was employed by SciKit-learn package v1.0.1 on the subjects in order to have an averaged VIP value for each species. Bar thickness reports the fold ratio (FR) value of the mean relative abundances for each species among the two cohorts, while an absent border indicates mean relative abundance of zero in the compared cohort. Mann-Whitney U test and Kruskal-Wallis tests were employed to assess significance for pairwise or multiple comparisons, respectively, considering a P value 50.05 as significant. All P values were corrected for multiple hypothesis testing using a two-stage Benjamini-Hochberg FDR at 10%. ROC curves were generated by a machine learning model employed in Sci-Kit learn package v1.0.1 trained on ICI response, using a polynomial support-vector machine (poly-SVM) with squared L2 penalty 8 and a train-test split with 5-fold cross-validation (StratifiedKFold, generating test sets with same distribution of classes and equal percentage of samples for each class). Venn diagrams were generated from selected species using the online software InteractiVenn, available at http://www.interactivenn.net/. All the analyses were made with Python v3.8.2 or R v4.1.2. Sankey diagram was generated with the Plotly library within Python v3.8.2

### Co-Abundance Network Analysis

Pearson matrices for network analysis (metric=Bray-Curtis, method=complete linkage) were generated on normalized and standardized data with in-house scripts (Python v3.8.2) and visualized with Gephi v1.0.9.2, as previously reported 9, 10. microbial species having a prevalence ≥2.5% were considered to generate the nodes within the final network, while a significant Pearson correlation coefficient and its related P value (after Benjamini-Hochberg FDR at 10%) was employed to obtain eight categories defining edge thickness 11. A leave-one-out cross-validation (LOOCV) method was employed by SciKit-learn package v1.0.1 on the discovery cohort in order to have an averaged P value for each correlation among two definite variables. Edges based on P values were thus pruned with two-stages FDR 10%, and a Q value 50.001 was considered as a higher threshold to start edge categorisation. Network analysis was performed with Gephi v1.0.9.2 (1), taking care of an optimized visual representation as proposed by current guidelines (Derosa et al. 2021; Spencer et al. 2021; Mager et al. 2020; Roberti et al. 2020; Overacre-Delgoffe et al. 2021), using Fruchterman Reingold then Force Atlas 2 algorithms (Park et al. 2022). Only connected nodes were retained in the final network, using the Gephi K-core filter (n=1): nodes passed from 536 to 404, with 2830 edges. Nodes were colored according to the cohort (NR or R) in which species harbored the highest mean relative abundance, after normalization and standardization. The degree value, measuring the in/out number of edges linked to a node, and the betweenness centrality, measuring how often a node appears on the shortest paths between pairs of nodes in a network, were computed with Gephi v1.0.9.2. Intra-network communities (Species Interacting Groups, SIGs) (Vétizou et al. 2015; Newsome et al. 2022) were retrieved using the Blondel community detection algorithm (Lee et al. 2022) by means of randomized composition and edge weights, with a resolution equal to 1 (McCulloch et al. 2022). Each microbial species could have been assigned to a different community with a community detection number (CDN). Nodes were colored even according to their SIG belonging. Network analysis taking care of microbial data compositionality (SPRING, Spice-Easi, SparCC) was performed by means of NetCoMi (Network Construction and Comparison for Microbiome Data) R package (Peschel et al. 2021).

### Determination of Species Interacting Groups (SIGs) and Toposcore Calculation

The membership of a species to a definite SIG was defined in three steps: i) 100 iterations of Blondel algorithm were performed with Gephi Toolkit v0.9.3 on the discovery cohort; ii) the variation coefficient (CV, standard deviation above mean) of the community assignation was computed for each species; iii) species having the same CV value were grouped into a SIG community and named after a greek letter. After computing the difference (AHigh-Low mOS) and fold-ratio (log 2FRHigh/Low mOS) in median OS for each microbial species (see the paragraph “Statistical analysis”), we averaged these values within each greek microbial community. Hence, out of 7 co-abundance communities networks representing the ecological community of the discovery cohort, we found a similar average difference (AHigh-Low mOS), fold-ratio (log 2FRHigh/Low mOS) and NR/R distribution among greek microbial communities mostly inhabited by NR species (α+β), and among two communities mostly inhabited by R species (γ+γ). Therefore, α+β communities were grouped in a new SIG1, while γ+δ communities were grouped into the new SIG2 (Table 3), and ε, ι, η became SIG3, SIG4, SIG5, respectively. A unifying parameter, called FRnormCOUNT, able to resume the topological and functional contraposition among SIG1 (96% NR) and SIG2 (97% R) was computed starting from SIG1 and SIG2 composition, based on the following equation [1], in which, for a definite individual, N_{SIG1} is the number of harbored species belonging to SIG1, while NSIG2 is the number of harbored species belonging to SIG2:

$$FRnormCOUNT = ( {N}_{SIG 1} / 40 ) / ( {N}_{SIG 2} / 34 ) \qquad [ 1 ]$$

This parameter goes from zero to infinite. A Kernel Density Estimation (KDE) of the FRnormCOUNT parameter for the discovery, validation, and total cohorts (see Table 4), was performed with kdeplot function within Seaborn v0.11.2, selecting a Gaussian kernel and the bandwidth, or standard deviation of the smoothing kernel, being optimally chosen by Kernel Density and GridSearchCV (cv=20) within SciKit-learn package v1.0.1. Three different regions, which cutoffs were computed with binning, cutpointR and Sirus R packages, resulted for FRnormCOUNT values: 1) a SIG2+ region (0<x<0.37), mostly harboring responders; 2) a Grey zone (0.37≤x<1.047), in which NR and R were equally represented; 3) a SIG1+ region (x≥1.047), mostly harboring non-responders. In order to resolve the Grey zone, we implemented the trichotomized approach previously published on Akkermansia muciniphila (Akk) relative abundances in NSCLC (Derosa et al. 2020; WO 2022/157207). Within this Grey zone, all of the patients harboring low Akk relative abundance (0<Akk≤4.799) were considered responders, while all of the patients harboring high Akk relative abundance (Akk≥4.8) and without Akk (Akk=0) at all were considered non-responders. With this information, we built a final score, named Toposcore, for categorizing unknown NSCLC patients for their OS at 12 months.

### Toposcore Algorithm

The experiments disclosed in Examples 8 to 13 below were done using the following Toposcore algotithm.

The scoring algorithm was developed based on the relative abundance of 536 metagenomics species (MGS) derived from 245 NSCLC cancer patients of the discovery cohort.

Each MGS was categorized as “low” or “high” if its relative abundance s or >median respectively. When a MGS had a majority of null abundances (i.e., median=0), this process matched the “absence” vs “presence” categorization. Cox Proportional Hazard (CoxPH) models were run on “overall survival” for each categorized MGS. A total of 266 MGS with a Hazard Ratio (HR)≤0.80 or 1.25 were retained in the model. The purpose of this selection was to discard MGS with HR close to 1, which are unlikely to participate in a diagnostic signature. Selected MGS were not necessarily significantly associated with OS as 1 might be contained in the 95% Confidence Interval (CI) of their HR. The Akkermansia muciniphila MGS was not considered in this screening because its relative abundance had a trichotomic distribution with no linear dose-effect relationship with patient prognosis as already reported in details (Lisa Derosa et al. 2021). Each pair of MGS was then analyzed by a Fisher'≤exact test on 2×2 contingency tables based on their Absence/Presence co-occurrences and scored by the by −log 10(p)×sign(OR−1) metrics, where p is the Fisher p-value and OR the Odds Ratio of the 2×2 table. This metrics defined a score proportional to the significance of the interaction between two MGS (−log 10(p)) that is negative in case of co-exclusion pattern (OR<1) or positive in case of co-occurrence (OR>1). Interactions with a Bonferroni-corrected p-value≤0.05 were retained for analysis. A total of 180 connected MGS were then clustered with Ward'≤method and Manhattan distance. The clustering tree was cut to obtain 7 clusters (C1 to C7). Two clusters (C5 and C6) contained 37 MGS mostly (95%) associated with OS<12 (HR≥1.25) that were used to define the SIG1 signature. Three clusters (C1, C2, C3) contained 45 MGS all associated with OS>12 months (HR≤0.80) that were used to define the SIG2 signature. In addition, interactions within SIG1 and SIG2 MGS were 99% and 100% positive respectively (co-occurrence patterns), while edges in-between SIG1 and SIG2 MGS were 98% negative (co-exclusion patterns), thus reflecting a significant and opposite topological separation.

Each patient of the discovery cohort was then scored with a S score computed as the difference of proportions between present (relative abundance>0) SIG2 and SIG1 MGS and scaled from 0 to 1: S=(#SIG2/45−#SIG1/37+1)/2. A score of 0 indicates that all MGS of the SIG1 signature have strictly positive relative abundances and all MGS of the SIG2 signature have null relative abundances. Conversely, a score of 1 indicates that all MGS of the SIG1 signature have null relative abundances and all MGS of the SIG2 signature have strictly positive relative abundances. A score of 0.5 indicates an equilibrium in proportions of present SIG1 and SIG2 MGS. The performance of this S score as predictor of OS12 was analyzed by a Receiver Operating Characteristic (ROC) analysis. Two scores, 0.5351 and 0.7911, were identified as local maxima of the Youden index (Specificity+Specificity−1) and were used as cutoffs to define three categories: SIG1+ if S≤0.5351, SIG2+ if S>0.7911, and “gray zone” otherwise.

The response is then predicted based on these categories: OS<12 in the SIG1+ category, and OS>12 in the SIG2+ category. The gray zone defines a range of scores where the relative proportions of present SIG2 and SIG1 MGS hardly discriminated survival outcomes. In this range, the Gram-anaerobic bacterium Akkermansia muciniphila SGB9226, for which a trichotomized distribution of the relative abundance was shown to correlate with OS (Lisa Derosa et al. 2022b), was used to predict response: OS>12 if Akkermansia muciniphila is low (in normal ranges), OS<12 if Akkermansia muciniphila is 0 or high (abnormal ranges). The performance of the predictor was assessed by Kaplan Meier (KM) analyses of predicted OS>12 vs. OS<12 in CoxPH analyses of OS in the discovery cohort, and repeated on several independent cohorts.

### Sirus Individual Prediction of Responsiveness to Immunotherapy

Sirus is a rule classification algorithm which is able to handle categorical and continuous variables, and, applying random forests plus decision trees, inherits a high accuracy and a stable structure, resulting in the highest reproducibility of prediction probability up to date (B6nard et al. 2021). We employed Sirus (R v4.1.2, package sirus) generating six different models (sirus.fit function) able to predict (sirus.predict function) the percentage of being NR for each NSCLC patient of validation (n=148) cohort, based on: i) species retrieved from SIG1 and SIG2 (modell, SIGSPECIES); ii) microbiota parameters computed on SIG1 and SIG2 (model2, SIGPARAMS, which encompass also Shannon and Richness metrics); iii) two selected parameters having the highest predictive combined value from the previous model2 (model3, FRnormCOUNT plus FRnormMEAN); iv) the selected parameter which retains the highest predictive value alone (model4, FRnormCOUNT); v) the combination of the previous model4 with the relative abundance of A. muciniphila (model5, FRnormCOUNT plus A. muciniphila relative abundances); vi) the solely A. muciniphila relative abundance values (model6, Akk). The parameter p0, which optimizes the number of rules that are used by Sirus to generate a model, was computed for each model by means of a default 10-fold cross validation (sirus.cv function).

### Random Forest (RF) Individual Prediction of Responsiveness to Immunotherapy

Random Forest classification was employed by means of Sci-Kit learn package v1.0.1 (sklearn.ensemble RandomForestClassifier, default settings except class_weight=‘balanced_subsample’, random_state=0, oob_score=True) and different models were generated on meta-variables (e.g., TOPOSCORE, AKK_TRICHO, SIG1/Grey/SIG2, LIPI, ECOGPS, PDL1, dNLR, Lymphocytes, BMI). The RF classifier was 5-fold cross-validated (sklearn.model_selection cross_val_score) in order to have an estimate of the final score (mean±SEM), and feature importance for each model (clf.feature_importances_) was reported after multiplication by 100. Features (thus, microbial species) deriving from the model with the highest cross-validated score (TOPOSCORE) with a value>1 were used to predict patients (clf.predict_proba) from the validation cohort (n=148) under the same previous RF settings and adding a train_test_split function (70% train, 30% test). RF predictions on the validation cohort were repeated 100 times and percentage values of being NR were reported as mean±SEM (Table 1).

### Machine Learning Prediction of Responsiveness to Immunotherapy.

First, we trained a RF classifier (SIAMCAT R package 1,000 estimator trees, with a minimum of 30% of features par tree)(“Microbiome Meta-Analysis and Cross-Disease Comparison Enabled by the SIAMCAT Machine Learning Toolbox|Genome Biology|Full Text” n.d.) on the discovery cohort in a 10-fold cross validation repeated 20 times, to assess AUC. Siamcat algorithm was used for the RF model, and its performance was measured by ROC curves and AUC value. Second, the 284 metagenome-assembled genomes (MAGs) from Guild1 and Guild2 found in our previous work were used as reference genomes to perform read recruitment analysis (Wu et al. 2022). The metagenomic reads were aligned to the MAGs using coverM with --min-read-aligned-percent 90-min-read-percent-identity 99. Abundances of the MAGs were used for the RF model, and its performance was measured by ROC curves and confusion matrix.

### Gene Pathway Functional Analyses

Functional potential analysis of the metagenomic samples was performed using HUMAnN 3.0 6 with default parameters. The MetaCyc “path abundance” profiles, expressed as RPK units, were analyzed by Dask v2021.10.0, in order to have a final matrix of pathways, both bulk and species-specific. In total, we identified 493 pathways (unclassified, unintegrated and unmapped excluded, 381 at 20% prevalence cutoff), in 393 samples from NSCLC discovery and validation cohorts. Subsequent statistical analysis was performed as described in the paragraph “Statistical analysis of metagenomic data”, taking into consideration only the pathways with a prevalence equal or higher than 20%, and the patients' categorization into NR and R following OS12 or into SIG1+ and SIG2+ following TOPOSCORE. In order to analyze the different pathways composition among SIG, each SIG species was measured for its contribution to each pathway, and RPK units results expressed as mean±SEM.

qPCR-based TOPOSCORE using Precision Microbiome Profiling (PMPTm).For each bacterium and archaea, we used the coords function of the pROC R package to determine the cut-off of PCR amplification using the Youden index allowing to reproduce the community detection of our reference measurement (shotgun metagenomics) with the best trade-off in terms of sensitivity and specificity. Due to the non-linear relationship between the PCR amplification and the presence status of Akkermansia spp. relative abundance (negative if Akk=0 or Akk>4.8, and positive if 0<Akk<4.8), we considered three categories: negative/low (Akk=0), positive (0<Akk<4.8), and negative/high (Akk≥4.8). We determined two PCR cutoffs according to the Youden index for multinomial response with the multiclass.roc function of the pROC R package. These analyses were realized using R v4.0.4.

### Code Availability

No unique software or computational code was created for this study. Code detailing implementation of established tools/pipelines are described in detail in the Method section and available upon request to the corresponding author. The entire analysis was programmed in R (ref: R Core Team (2022). R: A language and environment for statistical computing. R Foundation for Statistical Computing, Vienna, Austria).

TABLE 1
Comprehensive data from machine learning algorithms.

| | | | | INDIVIDUAL_PREDICTION | | | | |
|---|---|---|---|---|---|---|---|---|
| | Validation | Hits/ | | | Correct | Correct | | Uncorrect |
| | (L148) | Total | Coverage ^{d} | Correct | NR | R | Uncorrect | NR |
| SIRUS ^{a} | MODEL1 | NA | NA | NA | NA | NA | NA | NA |
| | MODEL2 | NA | NA | NA | NA | NA | NA | NA |
| | MODEL3 | 63/108 | 0.58 | 42 | 8 | 34 | 21 | 15 |
| | MODEL4 | 100/108 | 0.93 | 60 | 23 | 37 | 40 | 22 |
| | MODEL5 | 69/108 | 0.64 | 44 | 7 | 37 | 25 | 22 |
| | MODEL6 | 20/108 | 0.19 | 13 | 0 | 13 | 7 | 7 |
| RF ^{b} | RF | 57/108 | 0.53 | 33 | 8 | 25 | 24 | 5 |
| | TOPOSCORE | | | | | | | |
| CLINIC ^{c} | PD-L1 | 87/108 | 0.81 | 43 | 24 | 19 | 44 | 15 |
| | AkkTRICHO | 108/108 | 1 | 65 | 39 | 26 | 43 | 11 |
| | TOPOSCORE | 108/108 | 1 | 68 | 25 | 43 | 40 | 25 |

| | | | | PREDICTION | | |
|---|---|---|---|---|---|---|
| | INDIVIDUAL_PREDICTION | PREDICTION | | (coverage-adjusted)^{e} | | |
| | Uncorrect | Correct | Uncorrect | Correct | Uncorrect | |
| | R | (%) | (%) | (%) | (%) | ΔP_{C-U} ^{f} |
| SIRUS ^{a} | NA | NA | NA | NA | NA | NA |
| | NA | NA | NA | NA | NA | NA |
| | 6 | 66.7 | 33.3 | 38.9 | 19.4 | 19.5 |
| | 18 | 60 | 40 | 55.6 | 37 | 18.6 |
| | 3 | 63.8 | 36.2 | 40.7 | 23.1 | 17.6 |
| | 0 | 65 | 35 | 12 | 6.5 | 5.5 |
| RF ^{b} | 19 | 57.9 | 42.1 | 30.6 | 22.2 | 8.4 |
| CLINIC ^{c} | 29 | 49.4 | 50.6 | 39.8 | 40.7 | −0.9 |
| | 32 | 60.2 | 39.8 | 60.2 | 39.8 | 20.4 |
| | 15 | 63 | 37 | 63 | 37 | 26 |

^{a} models based on SIRUS pipeline (RF plus decision trees) as described in materials and methods.
^{b} most predictive Random Forest (RF) model as described in materials and methods.
^{c} models based on clinically-validated scores.
^{d} fraction of individual hits by each model on the total OS12 data available (see Table 10).
^{e}prediction percentage corrected for the coverage.
^{f} difference among correct and uncorrect prediction percentages.

### Example 1: Limitations in Predicting Clinical Outcome Across Cohorts and Cancer Types Using Single Metagenomic Species (MGS)

LUMIERE and ONCOBIOTICS have been two prospective observational studies recruiting 393 advanced inoperable NSCLC and 69 patients with renal cell cancer (RCC) in France and Canada since 2017. These cohorts of previously ICI-naïve or previously treated patients provided stool samples at baseline before ICI initiation, with detailed clinical data and comedications (Table 2). To study the prognostic impact of the gut microbiota composition on ICI responses in NSCLC and RCC, we performed shotgun metagenomics sequencing of frozen fecal samples in a first discovery cohort (enrolling subjects with NSCLC from 2017 to 2019), partially reported by Routy et al. (Routy et al. 2018; Derosa et al. 2020), and in a validation cohort (enrolling patients with NSCLC from 2019 up to 2021), partially reported by Derosa et al. (Derosa et al. 2021). A third prospective cohort of 61 ICI-naïve advanced NSCLC and 14 RCC patients was recently incremented (Table 2). Altogether, these prospective observational cohorts provide the largest assessment of the potential impact of the gut microbiome as a biomarker of response to ICI to date, allowing investigation of specific MGS, co-abundance networks and functions of clinical relevance for cancer immunotherapy across two different histotypes of cancer.

TABLE 2
Patients' characteristics in the two retrospective and the prospective cohorts.

| | | | Retrospective * | Discovery | Validation | |
|---|---|---|---|---|---|---|
| | | | (n = 393) | (n = 245) | (n = 148) | p-value° |
| Age (year) | Median (range) | | 64 (24-92) | 64 (32-85) | 66 (24-92) | 0.03 (continuous) |
| | <65 | yr | 197 (50) | 132 (54) | 65 (44) | 0.22 |
| | ≥65 to <75 | yr | 133 (34) | 76 (31) | 57 (39) | |
| | ≥75 | yr | 63 (16) | 37 (15) | 26 (18) | |
| Sex - no (%) | Male | | 259 (66) | 161 (66) | 98 (66) | 0.54 |
| | Female | | 134 (34) | 84 (34) | 50 (34) | |
| BMI, kg/cm² - no (%) | <25 | | 241 (63) | 160 (68) | 81 (56) | 0.03 |
| | [25-30[ | | 99 (26) | 56 (24) | 43 (30) | |
| | ≥30 | | 41 (11) | 21 (9) | 20 (14) | |
| | Unknown | | 12 | 8 | 4 | |
| ECOG-PS - no (%) | 0-1 | | 310 (86) | 178 (83) | 132 (90) | 0.11 |
| | 2 or more | | 50 (14) | 36 (17) | 14 (10) | |
| | Unknown | | 33 | 31 | 2 | |
| Smoking status - no. (%) | Never smoked | | 32 (8) | 19 (8) | 13 (9) | 0.83 |
| | Current/former | | 358 (92) | 226 (92) | 132 (90) | |
| | smoker | | | | | |
| | Unknown | | 3 | 0 | 3 | |
| Tumor histology - no. (%) | Squamous | | 89 (23) | 58 (24) | 31 (21) | 0.75 |
| | Non-squamous | | 304 (77) | 187 (76) | 117 (79) | |
| | Unknown | | — | — | — | |
| Tumor histology - no. (%) | Clear cell renal | | — | — | — | |
| | cell carcinomas | | | | | |
| | Non-clear cell | | — | — | — | |
| | renal cell | | | | | |
| | carcinomas | | | | | |
| PD-L1 status - no. (%) | <1% | | 71 (28) | 35 (27) | 36 (29) | 0.43 |
| | 1-49% | | 59 (23) | 27 (21) | 32 (25) | |
| | ≥50% | | 124 (49) | 66 (52) | 58 (46) | |
| | Unknown | | 139 | 117 | 22 | |
| IMDC - no. (%) | Good | | — | — | — | |
| | Intermediate | | — | — | — | |
| | Poor | | — | — | — | |
| | Unknown | | — | — | — | |
| Therapy line - no. (%) | Neoadjuvant/ | | 3 (1) | 0 (0) | 3 (2) | 0.01 |
| | adjuvant | | | | | |
| | First line | | 92 (23) | 50 (20) | 42 (28) | |
| | ≥Second line | | 298 (76) | 195 (80) | 103 (70) | |
| Therapy - no. (%) | Immunotherapy | | 382 (97) | 240 (98) | 142 (96) | <0.01 |
| | Immunotherapy and | | 11 (3) | 5 (2) | 6 (4) | |
| | Chemotherapy | | | | | |
| Previous therapy - no. (%) | Chemotherapy | | 292 (75) | 192 (79) | 100 (69) | 0.33 |
| | Tyrosine kinase | | 16 (4) | 13 (5) | 3 (2) | |
| | inhibitors | | | | | |
| | mTOR inhibitors +/− | | — | — | — | |
| | anti-VEGF | | | | | |
| | Others | | 35 (9) | 28 (12) | 7 (5) | |
| | Unknown | | 5 | 2 | 3 | |
| Antibiotics - no. (%) | Yes | | 82 (21) | 58 (24) | 24 (16) | 0.20 |
| | No | | 311 (79) | 187 (76) | 124 (84) | |
| | Unknown | | | | | |
| Follow-up - no. (%) | ≥12 | months | 334 (85) | 229 (93) | 105 (71) | |

| | | | Prospective | RCC | NSCLC |
|---|---|---|---|---|---|
| | | | (n = 144) | (n = 83) | (n = 61) |
| Age (year) | Median (range) | | 63 (31-85) | 61 (31-81) | 65 (50-85) |
| | <65 | yr | 75 (52) | 50 (60) | 25 (41) |
| | ≥65 to <75 | yr | 51 (35) | 23 (28) | 28 (46) |
| | ≥75 | yr | 18 (13) | 10 (12) | 8 (13) |
| Sex - no (%) | Male | | 97 (67) | 64 (77) | 33 (54) |
| | Female | | 47 (33) | 19 (23) | 28 (46) |
| BMI, kg/cm² - no (%) | <25 | | 67 (48) | 37 (46) | 30 (51) |
| | [25-30[ | | 61 (44) | 38 (48) | 23 (39) |
| | ≥30 | | 11 (8) | 5 (6) | 6 (10) |
| | Unknown | | 5 | 3 | 2 |
| ECOG-PS - no (%) | 0-1 | | 119 (85) | 71 (87) | 48 (80) |
| | 2 or more | | 23 (15) | 11 (13) | 12 (20) |
| | Unknown | | 2 | 1 | 1 |
| Smoking status - no. (%) | Never smoked | | 3 (2) | — | 3 (5) |
| | Current/former | | 55 (40) | — | 55 (93) |
| | smoker | | | | |
| | Unknown | | 3 | — | 3 |
| Tumor histology - no. (%) | Squamous | | 16 (11) | — | 16 (28) |
| | Non-squamous | | 42 (30) | — | 42 (72) |
| | Unknown | | 3 | — | 3 |
| Tumor histology - no. (%) | Clear cell renal | | 79 (56) | 79 (95) | — |
| | cell carcinomas | | | | |
| | Non-clear cell | | 4 (3) | 4 (5) | — |
| | renal cell | | | | |
| | carcinomas | | | | |
| PD-L1 status - no. (%) | <1% | | 24 (18) | — | 24 (47) |
| | 1-49% | | 14 (10) | — | 14 (27) |
| | ≥50% | | 13 (10) | — | 13 (25) |
| | Unknown | | 10 | — | 10 |
| IMDC - no. (%) | Good | | 29 (22) | 29 (35) | — |
| | Intermediate | | 39 (29) | 39 (48) | — |
| | Poor | | 14 (11) | 14 (17) | — |
| | Unknown | | 1 | 1 | — |
| Therapy line - no. (%) | Neoadjuvant/ | | — | — | — |
| | adjuvant | | | | |
| | First line | | 24 (17) | 10 (12) | 14 (28) |
| | ≥Second line | | 120 (83) | 73 (88) | 47 (92) |
| Therapy - no. (%) | Immunotherapy | | 144 (100) | 83 (100) | 61 (100) |
| | Immunotherapy and | | — | — | — |
| | Chemotherapy | | | | |
| Previous therapy - no. (%) | Chemotherapy | | 61 (42) | — | 61 (100) |
| | Tyrosine kinase | | 65 (45) | 65 (78) | — |
| | inhibitors | | | | |
| | mTOR inhibitors +/− | | 8 (5) | 8 (10) | — |
| | anti-VEGF | | | | |
| | Others | | — | — | — |
| | Unknown | | — | — | — |
| Antibiotics - no. (%) | Yes | | 23 (16) | 10 (12) | 13 (22) |
| | No | | 120 (84) | 73 (88) | 47 (78) |
| | Unknown | | 1 | — | 1 |
| Follow-up - no. (%) | ≥12 | months | 128 (89) | 80 (95) | 48 (79) |

°P-value between Discovery and Validation cohorts.
RCC: renal cell carcinoma; NSCLC: non-small cell lung cancer; BMI: body mass index; ECOG: Eastern Cooperative Oncology Group Performance Status; IMDC: International Metastatic Database Consortium Risk Model for Metastatic Renal Cell Carcinoma.

We first analyzed whether the fecal taxonomic composition at baseline in the discovery cohort composed of 245 NSCLC patients would predict overall survival beyond 12 months (OS>12) during a first-, ≥second line (≥2L) therapy with anti-PD-1 or anti-PD-L1 antibodies (Abs). To characterize differences in microbial composition between patient groups achieving OS<12 (non-responders (NR), n=112) or OS>12 (responders (R), n=118), we monitored the variations in stool microbial alpha diversity and performed principal coordinate analyses (PCoA) of microbial beta diversity distances (Bray-Curtis). Of note, 15 patients did not reach a 12 month-minimal follow up and could not be included in this analysis. Alpha diversity (Shannon index) was significantly different in the two groups (FIG. 1A, upper panel). Next, we utilized an unsupervised PCoA to explore putative differences in general microbiota composition between short- and long-term survivors, finding a clear difference between the two groups (FIG. 1A, middle panel, p=0.0039). To determine the relative contribution of each microbial species abundance at baseline to the observed group separation, MGS were ordered according to their variable importance (VIP) score (FIG. 1A, right panel) which relies on the supervised Partial Least Squares Discriminant Analysis (PLS-DA). Some of the significant MGS that were associated with OS<12 were already described in antibiotic-treated or poor prognosis cancer patients (such as Hungatella hathewayi, Clostridium innocuum, Streptococcus anginosus or Actinomyces graevenitzii) (Derosa et al. 2020; Tsay et al. 2020).

Next, we turned to the validation cohort of 148 patients with NSCLC in which slightly more therapy-naïve patients were enrolled than in the discovery cohort (Table 2). However, neither the alpha diversity (FIG. 1B, upper panel, p=0.8389) nor the beta-diversity (FIG. 1B, middle panel, p=0.1239) could segregate OS<12 or OS>12 subsets. Of note, 40 patients did not reach a 12 month-minimal follow up and could not be included in this analysis. Considering strictly MGS identified using the MetaPhlAn4 pipeline commonly found in both cohorts (discovery and validation), we only found two MGS associated with OS>12, namely Roseburia intestinalis and Anaerostipes hadrus. The Cox regression analysis of the effect of the relative abundance of either one of these two microbial hits on OS (considering the “high”≥0 and the “low”<0 of normalized and standardized relative abundance values as cut-off) confirmed that only R. intestinalis and A. hadrus significantly predicted long term clinical benefit to PD-1 blockade, in both, the discovery (FIG. 2A) and the validation cohorts (FIG. 1C). Despite these apparently encouraging results, the polinomial Support Vector Machine (poly-SVM) Receiver Operating Characteristic (ROC) curves (Chang et Lin 2011) measuring the performance of the relative abundance of these MGS to classify patients into OS<12 or ≥12 indicated AUC values around 0.5, suggesting that this model was not able to accurately predict patient prognosis (FIG. 1D). Moreover, when considering another histotype of cancer such as a cohort of RCC patients, the relative abundance of Anaerostipes hadrus spp failed to predict long-term survivors (FIG. 1E). In fact, both MGS correspond to highly prevalent (around 70%) and relatively abundant species (FIG. 1B-C), suggesting that they are necessary but not sufficient to accurately predict immunosensitivity.

Hence, as already discussed (McCulloch et al. 2022), despite large and homogeneous cohorts handled by the same investigators using a clinically relevant endpoint (OS at 12 months), and optimized machine learning algorithms, we failed at identifying a prototypical MGS fingerprint robustly predicting clinical benefit to PD-1 blockade.

The above results were actualized in 2023 after more patients were recruited in LUMIERE and ONCOBIOTICS prospective observational studies (NCT03084471), reaching 499 advanced NSCLC in France and Canada since 2017 and 83 renal cell cancer (RCC) in France. The actualized alpha diversity (Shannon index), differences in general microbiota composition between short- and long-term survivors and relative contribution of each microbial species abundance at baseline in patient subgroups [OS<12 months vs. OS>12 months] are shown in Discovery (FIG. 1F) and Validation (FIG. 1G) cohorts.

### Example 2: Building Co-Abundance Networks within the Microbial Ecosystem of Patients with NSCLC

Resource and niche competition, as well as metabolic cross-feeding are among the main drivers of microbial community assembly (Friedman et al. 2017; Clark et al. 2021; Sanchez-Gorostiaga et al. 2019). Nonetheless, the degree to which these forces are reflected in the composition of the intestinal communities of long-term responders (R) or non-responders (NR) has not been investigated to date. Here we used genome-scale species modeling to assess cooperation potential in large species interacting groups across thousands of MGS in the discovery cohort, which we attempted to corroborate in the validation cohort. Only MGS with a prevalence ≥2.5% were considered when generating the nodes within the final network, while a significant Pearson correlation coefficient and its related p-value (after Benjamini-Hochberg FDR at 10%) was employed to obtain categories defining edge thickness (Li et al. 2008). A leave-one-out cross-validation procedure was employed on the discovery cohort in order to have an averaged p-value for each correlation among two definite variables. This analysis revealed seven distinct communities apostrophed “SIG” (“species interacting group”) annotated with Greek letters, clustering at distant or opposite ends in a trade-off between competition and cooperation to predict OS at 12 months (Tables 3-4).

TABLE 3
List of bacteria within each community found with Pearson network

SIG1

| SIG_alfa | SIG_beta |
|---|---|
| Alloscardovia_omnicolens | Eggerthella_lenta |
| Bifidobacterium_dentium | Enterocloster_aldensis |
| Campylobacter_concisus | Enterocloster_asparagiformis |
| Clostridium_perfringens | Enterocloster_bolteae |
| Dialister_invisus | Erysipelatoclostridium_ramosum |
| Enterococcus_durans | Faecalimonas_umbilicata |
| Enterococcus_faecalis | Gordonibacter_urolithinfaciens |
| Enterococcus_faecium | |
| Haemophilus_parainfluenzae | |
| Klebsiella_pneumoniae | |
| Lacticaseibacillus_paracasei | |
| Lacticaseibacillus_rhamnosus | |
| Lactobacillus_delbrueckii | |
| Lactobacillus_gasseri | |
| Lactobacillus_vaginalis | |
| Lactococcus_lactis | |
| Lactococcus_laudensis | |
| Ligilactobacillus_salivarius | |
| Limosilactobacillus_fermentum | |
| Limosilactobacillus_oris | |
| Megasphaera_micronuciformis | |
| Mogibacterium_diversum | |
| Scardovia_wiggsiae | |
| Streptococcus_anginosus | |
| Streptococcus_gordonii | |
| Streptococcus_infantis | |
| Streptococcus_mutans | |
| Streptococcus_parasanguinis | |
| Streptococcus_salivarius | |
| Veillonella_atypica | |
| Veillonella_dispar | |
| Veillonella_parvula | |
| Veillonella_rogosae | |

SIG2

| SIG-gamma | SIG-delta |
|---|---|
| Candidatus_Cibiobacter_qucibialis | Adlercreutzia_equolifaciens |
| Clostridiales_bacterium_KLE1615 | Agathobaculum_butyriciproducens |
| Dorea_formicigenerans | Anaerobutyricum_hallii |
| Dorea_longicatena | Anaerostipes_hadrus |
| Eubacterium_ventriosum | Blautia_faecis |
| Faecalibacillus_intestinalis | Blautia_massiliensis |
| Holdemania_filiformis | Blautia_obeum |
| Lachnospira_eligens | Blautia_wexlerae |
| Lacrimispora_celerecrescens | Clostridia_unclassified_SGB4447 |
| Parasutterella_excrementihominis | Clostridiaceae_bacterium |
| | Clostridium_sp_AF34_10BH |
| | Clostridium_sp_AF36_4 |
| | Eubacteriaceae_bacterium |
| | Eubacterium_rectale |
| | Faecalibacterium_prausnitzii |
| | Fusicatenibacter_saccharivorans |
| | Lachnospira_pectinoschiza |
| | Lachnospiraceae_bacterium |
| | Roseburia_faecis |
| | Roseburia_hominis |
| | Roseburia_intestinalis |
| | Roseburia_inulinivorans |
| | Ruminococcus_bicirculans |
| | Ruminococcus_lactaris |

SIG3-epsilon
Candidatus_Allobutyricicoccus_pentlandensis
Candidatus_Gallimonas_merdae
Candidatus_Heteroclostridium_caecigallinarum
Candidatus_Heteroruminococcus_faecigallinarum
Candidatus_Heteroscilispira_lomanii
Candidatus_Neoclostridium_roslinense
Clostridia_unclassified_SGB15402
Clostridia_unclassified_SGB66170
Desulfovibrio_sp_PG_178_WT_4
Firmicutes_bacterium
Intestinimonas_massiliensis
Rikenellaceae_bacterium_DSM_108975

| SIG4-zeta | SIG5-eta |
|---|---|
| Alistipes_inops | Alistipes_senegalensis |
| Anaeromassilibacillus_sp_An250 | Bacteroides_togonis |
| Candidatus_Alloscillospira_gallinarum | Butyricimonas_faecihominis |
| Candidatus_Aristotella_avistercoris | Catenibacterium_sp_AM22_15 |
| Candidatus_Avimonas_narfia | Clostridia_unclassified_SGB4367 |
| Candidatus_Howiella_intestinavium | Clostridiales_Family_XIII_bacterium |
| Candidatus_Metaruminococcus_gallistercoris | WCA_MUC_591_APC_4B |
| Candidatus_Neochristensenella_gallicola | Collinsella_bouchesdurhonensis |
| Candidatus_Paralachnospira_caecorum | Eggerthellaceae_unclassified_SGB14322 |
| Candidatus_Pseudobutyricicoccus_lothianensis | Eubacterium_sp_AM28_29 |
| Candidatus_Pseudolachnospira_avium | Holdemanella_biformis |
| Candidatus_Schneewindia_gallinarum | Leuconostoc_mesenteroides |
| Catenibacillus_scindens | Methanomassiliicoccales_archaeon |
| Clostridia_bacterium_12CBH8 | Phascolarctobacterium_succinatutens |
| Clostridia_unclassified_SGB14844 | Prevotella_copri_clade_A |
| Clostridiales_bacterium_CHKCI006 | Prevotella_copri_clade_B |
| Clostridiales_unclassified_SGB15150 | Prevotella_copri_clade_C |
| Clostridium_sp_Marseille_P3244 | Prevotella_SGB1680 |
| Eubacteriaceae_bacterium_CHKCI004 | Prevotella_sp_P4_51 |
| Flavonifractor_sp_An10 | Senegalimassilia_anaerobia |
| Intestinimonas_timonensis | Senegalimassilia_faecalis |
| Lachnoclostridium_phocaeense | Slackia_isoflavoniconvertens |
| Lachnoclostridium_SGB4598 | |
| Lachnoclostridium_sp_An131 | |
| Lachnoclostridium_sp_An138 | |
| Massilioclostridium_coli | |
| Massilistercora_timonensis | |
| Merdibacter_massiliensis | |
| Merdimonas_faecis | |
| Olsenella_SGB14390 | |
| Pseudoflavonifractor_capillosus | |
| Pseudoflavonifractor_SGB15156 | |
| Ruminococcus_bromii | |
| Subdoligranulum_SGB15305 | |

TABLE 4
SIG1 and SIG2 bacteria and their association with OS.

| | mOS | | OS12 status^{d} | | | | | | | |
|---|---|---|---|---|---|---|---|---|---|---|
| | High- | Log2 | | | NR | R | | | High- | Log2 |
| Community^{a} | Low^{b} | (High/Low)^{c} | NR | R | (%) | (%) | SIGs^{e} | #species^{f} | Low^{g} | (High/Low)^{h} |
| alfa | −5.834 | −0.844 | 31 | 2 | 93.9 | 6.1 | SIG1 | 40 | −10.292 | −1.314 |
| beta | −4.458 | −0.470 | 7 | 0 | 100 | 0 | | | | |
| gamma | 5.364 | 0.577 | 1 | 9 | 10 | 90 | SIG2 | 34 | 10.480 | 1.142 |
| delta | 5.115 | 0.566 | 0 | 24 | 0 | 100 | | | | |
| epsilon | −1.389 | −0.240 | 8 | 4 | 66.7 | 33.3 | SIG3 | 12 | | |
| zeta | 1.151 | −0.005 | 14 | 20 | 41.2 | 58.8 | SIG4 | 34 | | |
| eta | 5.049 | 0.405 | 10 | 11 | 47.6 | 52.4 | SIG5 | 21 | | |

^{a}community as defined by Blondel algorithm (Louvain method); ^{b}community-averaged difference of the mOS (expressed in months) among the high and low normalized and standardized microbial relative abundances; ^{c}community-averaged 2-base logarithm of the high/low ratio of the mOS among the high and low normalized and standardized microbial relative abundances; ^{d}count and percentage of species with higher relative abundance in NR and responder (R) following OS12; ^{e}Species Interacting Group (SIG) definition after merging alfa-beta and gamma-delta communities; ^{f}number of species within each SIG; ^{g}cumulative loss or gain of mOS (expressed in months) for SIG1 and SIG2; ^{h}cumulative loss or gain of mOS ratio for SIG1 and SIG2.

Driven by this observation, we employed Cox regression survival analysis and Kaplan-Meier curves (R packages survival, survminer, Rcpp), computing the difference (ΔHigh-Low mOS) and fold-ratio (log 2FRHigh/Low mOS) in median OS for the “high”(≥0) and “low”(<0) normalized/standardized values of the relative abundance for each MGS contained within each of the seven SIGs, as we performed for A. hadrus and R. intestinalis (Table 4). Hence, out of 7 co-abundance networks representing the ecological community of the discovery cohort, we found similar average and fold-ratio differences among microbial communities mostly inhabited by NR species (α+β), and among two communities mostly inhabited by R species (γ+β). Therefore, α+β and γ+δ communities were grouped in SIG1 and SIG2 respectively (Tables 3-4). Instead of Pearson-based correlations to establish the co-abundance network, we used the semi-parametric rank-based approach to correlation estimation for INference in Graphical models (SPRING) of statistical microbial association networks from quantitative microbiome data (that can naturally deal with the excess zeros in the data). We found similar SIG compositions utilizing three alternative computed networks (SPRING, SPIEC-EASI, CCREPE models) (not shown). SIG1 and SIG2 harbored a different microbial composition in thus far that 5% and 95% of SIG1 microbial species were associated with OS>12 or <12 respectively, while 97% and 3% for SIG2 were associated with OS>12 or <12 respectively (p<0.0001) (Table 5).

TABLE 5
Percentage distribution of microbial species OS-
related within SIG1 and SIG2, discovery cohort.

| | | | |
|---|---|---|---|
| Discovery | OS < 12 | OS ≥ 12 | p-value |

| | | | | | |
|---|---|---|---|---|---|
| SIG1 | 38 | (95%) | 2 | (5%) | <0.0001 |
| SIG2 | 1 | (3%) | 33 | (97%) | |

χ2 statistics summarizing the numbers of MGS associated with responder (OS > 12 months) or non-responder (NR, OS < 12 months) patients in the microbial network for the discovery cohort (n = 245) using Pearson matrices generated on normalized and standardized relative abundances of MGS having a prevalence ≥2.5%.

Hence, SIG1 and SIG2 were composed of 40 “harmful” and 34 “beneficial” microbial species, respectively, because SIG1- or SIG2-related MGS led to a cumulative loss or gain in median OS of more than 10 months, respectively (Table 4). Indeed, SIG1 contained members belonging to the Enterocloster genus, and Streptococcaceae, Veillonellaceae and Lactobacillaceae families that were already associated with dismal prognosis or immunoresistant patient populations (Spencer et al. 2021; Lee et al. 2022; McCulloch et al. 2022; Tsay et al. 2020; Yonekura et al. 2021). Conversely, SIG2 assembled Lachnospiraceae (species from the genus Blautia, Roseburia, Dorea, Eubacterium), and Oscillospiraceae family members (Faecalibacterium prausnitzii, Ruminococcus bicirculans and R. lactaris), which are associated with general health and favorable clinical responses to ICI (Gopalakrishnan et al. 2018; Messaoudene et al. 2022).

Next, we reduced this whole-population-based network down to a monodimensional score by computing a SIG1/SIG2 fold-ratio of normalized microbial counts in which, for a given patient, N_{SIG1} is the number of prevalent species belonging to SIG1 divided by 40, while N_{SIG2} is the number of prevalent species belonging to SIG2 divided by 34 in the MGS available for that particular patient (i.e., FRnormCOUNT=(N_{SIG1}/40)/(N_{SIG2}/34)). Theoretically, this value goes from zero to infinite. A Kernel Density Estimation (KDE) of the FRnormCOUNT parameter was performed for the discovery cohort in order to estimate the boundaries that better segregate NR and R distributions (p=0.00023) (Table 6, FIG. 3). Three different regions resulted from the distribution of FRnormCOUNT values, identifying a SIG2 region (0<FRnormCOUNT<0.37), mostly harboring R (63% of patients with OS>12), an intermediate “Grey zone” (0.37≤FRnormCOUNT<1.047), in which NR and R were equally represented, and a SIG1 region (FRnormCOUNT≥1.047), mostly harboring NR (77% of patients with OS<12).

The Cox regression analysis of the impact of the FRnormCOUNT on OS highlighted that patients with a FRnormCOUNT falling within the SIG2 exhibited a significantly prolonged clinical benefit to PD-1 blockade than patients falling into SIG1 or Grey zone (FIG. 4A). As explained above, the FRnormCOUNT is based on the prevalence (presence or absence) of each SIG commensal, but not on the relative abundance (rel. abund.). However, this did not apply to one Gram negative anaerobic bacterium harboring regulatory and metabolic functions, Akkermansia muciniphila SGB9226 (Akk), for which a trichotomized distribution of the relative abundance best correlated with OS, as already reported (Derosa et al. 2021). Hence, to solve the uncertainty of the “Grey zone”, we segregated patients according to the trichotomized distribution of Akk relative abundance (Derosa et al. 2021; WO 2022/157207) (FIG. 4B). Patients who harbored physiological Akk levels (0<Akk≤4.799, Akk^{L}) represented 29% of the Grey zone and were considered R, while patients devoid of (Akk⁰) or harboring high Akk levels (Akk≥4.8, Akk^{H}) and constituting 53% and 17% of the Grey zone respectively were considered NR (Table 6).

Finally, with both pieces of information (FRnormCOUNT+Akk level), we built a final categorical score of “immunoresistance-related dysbiosis”, named “TOPOSCORE”, to classify NSCLC patients into two risk categories, either R (predicting OS>12 months) or NR (predicting OS<12 months) (FIG. 4B, Table 6). Indeed, the Cox regression analysis of the impact of the TOPOSCORE on OS highlighted that patients with a TOPOSCORE falling within the SIG2+ Grey zone Akk^{L} (apostrophed “SIG2+” henceforth) exhibited a significantly prolonged clinical benefit to PD-1 blockade compared with patients with a TOPOSCORE falling within the SIG1+ Grey zone Akk^{0/H} (apostrophed “SIG1+” henceforth) in multivariate analyses (Table 6, FIG. 4C-D). Of note, the TOPOSCORE classifier represented a prognosis marker independent from the ECOG performance status, LIPI score and PD-L1 expression in the discovery NSCLC cohorts.

TABLE 6
Distribution of retrospective NSCLC cohorts within Toposcore regions.

| | | | | | Patients with | | | p-value |
|---|---|---|---|---|---|---|---|---|
| | | | | Patients - | follow-up >12 | OS <12- | OS >12- | (Chi- |
| Cohort | | Toposcore Category | | no (%) | months | no (%)* | no (%)* | square) |
| NSCLC | Discovery | Toposcore | SIG 2 | 135 (55) | 126 (55) | 46 (37) | 79 (63) | <0.0001** |
| | (n = 245) | SIG2+ | Grey Akk^{L} | 28 (11) | 27 (12) | 11 (41) | 16 (59) | |
| | | Toposcore | SIG 1 | 27 (11) | 26 (11) | 20 (77) | 6 (23) | |
| | | SIG1+ | Grey Akk⁰ | 44 (18) | 40 (17) | 24 (60) | 16 (40) | |
| | | | Grey Akk^{H} | 11 (5) | 11 (5) | 10 (91) | 1 (9) | |
| | Validation | Toposcore | SIG 2 | 85 (57) | 61 (56) | 22 (36) | 39 (64) | 0.0096** |
| | (n = 148) | SIG2+ | Grey Akk^{L} | 10 (7) | 7 (7) | 3 (43) | 4 (57) | |
| | | Toposcore | SIG 1 | 17 (11) | 11 (10) | 6 (55) | 5 (45) | |
| | | SIG1+ | Grey Akk⁰ | 25 (17) | 21 (20) | 13 (62) | 8 (38) | |
| | | | Grey Akk^{H} | 11 (8) | 8 (7) | 6 (75) | 2 (25) | |

*Percentage calculated in each category;
**Comparing SIG2 and Grey Akk^{L} vs SIG1 and Grey Akk⁰ and Akk^{H}.

Applying the same network algorithm in the validation cohort of 148 NSCLC patients, we observed a similar co-abundance network, with 30% and 70% of SIG1 bacteria that were associated with OS>12 or <12 respectively, while 74% and 26% for SIG2 were associated with OS>12 or <12 respectively (p=0.0019) (Table 7). Of note, 15 (out of 40) and 23 (out of 34) MGS were shared with the discovery set for SIG1 and SIG2 respectively (FIG. 5, Table 8).

TABLE 7
Percentage distribution of microbial species OS-
related within SIG1 and SIG2, validation cohort.

| | | | |
|---|---|---|---|
| Validation | OS < 12 | OS ≥ 12 | p-value |

| | | | | | |
|---|---|---|---|---|---|
| SIG1 | 14 | (70%) | 6 | (30%) | 0.0019 |
| SIG2 | 8 | (26%) | 23 | (74%) | |

χ2 statistics summarizing the numbers of MGS associated with responder (OS > 12 months) or non-responder (NR, OS < 12 months) patients in the microbial network for the validation cohort (n = 148) using Pearson matrices generated on normalized and standardized relative abundances of MGS having a prevalence ≥2.5%.

TABLE 8
Commonalities in bacteria species between
Discovery and Validation cohorts.

| SIG1 | SIG2 |
|---|---|
| Alloscardovia omnicolens | Agathobaculum butyriciproducens |
| Bifidobacterium dentium | Anaerostipes hadrus |
| Haemophilus parainfluenzae | Blautia faecis |
| Lactobacillus gasseri | Blautia massiliensis |
| Limosilactobacillus fermentum | Blautia obeum |
| Megasphaera micronuciformis | Candidatus Cibiobacter_qucibialis |
| Scardovia wiggsiae | Clostridiaceae bacterium |
| Streptococcus anginosus | Clostridiales bacterium KLE1615 |
| Streptococcus infantis | Clostridium sp AF34 10BH |
| Streptococcus parasanguinis | Dorea formicigenerans |
| Streptococcus salivarius | Dorea longicatena |
| Veillonella atypica | Eubacterium rectale |
| Veillonella dispar | Eubacterium ventriosum |
| Veillonella parvula | Faecalibacillus intestinalis |
| Veillonella rogosae | Faecalibacterium prausnitzii |
| | Fusicatenibacter saccharivorans |
| | Lachnospira eligens |
| | Lachnospira pectinoschiza |
| | Lachnospiraceae bacterium |
| | Roseburia faecis |
| | Roseburia hominis |
| | Roseburia intestinalis |
| | Rosebuia inulinivorans |

The boundaries of the KDE for the validation cohort were also able to accurately segregate NR and R within the 148 patients (FIG. 6A, p=0.0486). As shown for the discovery cohort, the Cox regression analysis of the impact of the TOPOSCORE on OS validated that the “SIG2+” category of patients exhibited a significantly prolonged clinical benefit to PD-1 blockade compared with the “SIG1+” subgroup (Table 6, FIG. 61B, p=0.0153). Here again, the TOPOSCORE classifier represented a prognosis marker independent from the ECOG performance status, LIPI score and PD-L1 expression in the validation NSCLC cohort (FIG. 6C). Importantly, pooling all NSCLC patients with available PD-L1 expression (n=304), we could demonstrate the added value of the TOPOSCORE essentially in PD-L1 positive NSCLC tumor-bearing hosts (FIG. 6D, p=0.003).

In conclusion, the TOPOSCORE identified on a per capita basis an “immunoresistance-related dysbiosis” on an individual basis in about 33% of patients, the majority (two thirds) among whom were ICI resistant, and 67% cases devoid of dysbiosis, two thirds among whom were ICI responders (Table 6). The prognostic value of the TOPOSCORE was demonstrated either in treatment-naïve or previously treated patients or in patients treated with ICI monotherapy (FIG. 7A-B).

The TOPOSCORE thus provides an individual diagnosis tool evaluating the risk of resistance to PD-1 blockade for advanced NSCLC patients.

### Example 3: Prospective Validation of the TOPOSCORE in Other Cohorts of Cancers Amenable to PD-1 Blockade

We next applied the TOPOSCORE to a new prospective cohort of NSCLC (n=61) and RCC (n=83) treated with ICI (described in Table 2), for which baseline MGS and a >6 months clinical follow-up was available. The percentage of patients falling into SIG2+ and SIG1+ for this pooled cohort was 76% and 24%, respectively (FIG. 8A). The Cox regression analysis of the impact of the TOPOSCORE on OS confirmed that the “SIG2+” subset of patients harbor a prolonged survival compared with the “SIG1+” subset (Table 6, FIG. 8A, p=0.0595).

Of note, considering only the prospective cohort of RCC, the TOPOSCORE classifier outperformed the IMDC score (FIG. 8B).

The Cox regression analysis on the OS of the NSCLC overall cohort (n=393 patients) confirmed that patients with a TOPOSCORE falling within the SIG2+ Grey Akk^{L} region exhibited a significantly (p<0.0001) prolonged clinical benefit to PD-1 blockade compared with individuals harboring a TOPOSCORE within SIG1+ Grey Akk^{0/H} region (FIG. 8C).

Combining the results from all NSCLC patients (n=382 with follow-up >12 months), we found that the sensitivity (Se), specificity (Sp), positive predictive value (PPV), and negative predictive value (NPV) of the TOPOSCORE are 76.8%, 48.0%, 62.7% and 64.7%, respectively (Table 9).

TABLE 9
Calculation of sensitivity, specificity, positive and negative
predictive values for the toposcore in NSCLC patients.

Clinical outcome,
OS12 (N = 382)

| | | R | NR |
|---|---|---|---|
| TOPOSCORE | SIG2+ Grey Akk^{L} | A = 156 | B = 93 |
| Result | SIG2+ | 133 | 74 |
| | Grey Akk^{L} | 23 | 19 |
| | SIG1+ Gray | C = 47 | D = 86 |
| | Akk^{H/0} | | |
| | SIG1+ | 15 | 28 |
| | Gray Akk^{H/0} | 32 | 58 |

Sensitivity A/A + C = 76.8%.
Specificity D/D + B = 48.0%.
Positive Predictive Value A/A + B = 62.7%.
Negative Predictive Value D/D + C = 64.7%.

To apply the TOPOSCORE to healthy individuals (HV), we computed the metagenomes from the public databases (n=5345) and utilized the MetaphLAn4 pipeline. To analyze the differences in the taxonomic stool composition between healthy subjects and advanced NSCLC patients (segregated into 242 R and 176 NR within the whole cohort for whom we had a follow up >12 months), we performed principal coordinate analyses of microbial beta diversity distances that unveiled significant distances using Bray-Curtis between HV and cancer groups (FIG. 9A). To determine the relative contribution of each microbial species abundance at baseline to the observed three group separation, MGS were ordered according to their VIP score which relied on the supervised PLS-DA (FIG. 9B). Not surprisingly, most significant MGS featured in Table 4 and the TOPOSCORE. In fact, the FRnormCOUNT applied to HV highlighted that 88.5% were SIG2, 10.4% fell in the Grey zone and 1.05% were SIG1. Using the Akk trichotomic distribution, we could calculate that 7.8% of HV are SIG1+ and could be considered a priori as “immunoresistant” (FIG. 9C).

In conclusion, the TOPOSCORE represents a robust biomarker predicting immunosensitivity and immunoresistance to ICI across two different cancer populations on an individual basis.

### Example 4: Machine Learning Prediction Algorithms

We moved further with a comparison of our TOPOSCORE with two machine-learning (ML) algorithms, namely Random Forest (RF) and Stable and Interpretable RUle Set (Sirus) («A predictive index for health status using species-level gut microbiome profiling|Nature Communications»s. d.) in order to build predictive models on NSCLC discovery cohort (n=245) (not shown). We built six different Sirus models based on all SIG1 and SIG2 microbial species, topological parameters, FRnormCOUNT and its combination with Akk, finding out poor predictive power and coverage based on individual predictions on the validation cohort (n=148) (Table 10). Only the FRnormCOUNT parameter (Sirus model 4) held a better coverage (93%) with a better discrepancy among correct and uncorrect prediction percentages (APC-U, 19%). Applying the RF algorithm on several meta-variables provided the highest cross-validated predictive power for TOPOSCORE (81.6±6.4%), but its validation was not meaningful, with a coverage of 53% and a ΔPC-U of around 8% (not shown). Thus, the referenced ML algorithms gave poor results in terms of individual prediction compared to TOPOSCORE, which, in turn, holds a 100% patients' hits and the highest ΔPC-U, with a discrepancy of 26% (Table 10).

TABLE 10
Toposcore and alternative machine-learning algorithms
predicting response or resistance to ICI.

| | | | PREDICTION of | | |
|---|---|---|---|---|---|
| | Models based | | Validation cohort (L148) | | |
| | on Discovery | | coverage-adjusted)^{e} | | |
| | cohort | Coverage | Correct | Uncorrect | |
| | (L245) | (%)^{d} | (C, %) | (U, %) | ΔP_{C-U}^{f} |
| SIRUS^{a} | MODEL1 | NA | NA | NA | NA |
| | MODEL2 | NA | NA | NA | NA |
| | MODEL3 | 58 | 38.9 | 19.4 | 19.5 |
| | MODEL4 | 93 | 55.6 | 37 | 18.6 |
| | MODEL5 | 64 | 40.7 | 23.1 | 17.6 |
| | MODEL6 | 19 | 12 | 6.5 | 5.5 |
| RF^{b} | RF_TOPOSCORE | 53 | 30.6 | 22.2 | 8.4 |
| CLINIC^{c} | PD-L1 | 81 | 39.8 | 40.7 | −0.9 |
| | AkkTRICHO | 100 | 60.2 | 39.8 | 20.4 |
| | TOPOSCORE | 100 | 63 | 37 | 26 |

^{a}models based on SIRUS pipeline (RF plus decision trees) as described in materials and methods; ^{b}most predictive Random Forest (RF) model as described in materials and methods; ^{c}models based on clinically-validated scores; ^{d}fraction of individual hits by each model on the total OS12 data available (see Table 1); ^{e}prediction percentage corrected for the coverage; ^{f}difference among correct and uncorrect prediction percentages.

### Example 5: Functional Metabolic Fingerprints Associated with SIGs

Next, we explored putative mechanisms whereby the taxonomic fecal composition may influence ICI response, based on organism-specific gene hits annotated according to the Kyoto Encyclopedia of Genes and Genomes Orthology in each NSCLC cohort. Following these annotations, reads from each sample were reconstructed into metabolic pathways using the MetaCyc hierarchy of pathway classifications, by means of the HUMAnN 3.0 pipeline. We retrieved 493 pathways (unclassified excluded, 381 at 20% prevalence cutoff), with 372 pathways common among discovery and validation cohorts (not shown). PCoA based on Bray Curtis Dissimilarity Index showed significant compositional differences in the functional pathways across sample types among NR (OS<12 months) versus R patients (OS>12 months), in both the discovery and the validation cohorts (FIG. 10A-B). Analyzing the VIP plots derived from supervised PLS-DA of retrieved pathways, six metabolic traits were found to be common among both cohorts (not shown). The PLS-DA VIP analysis indicated that patients who had OS>12 showed pathway enrichment for the biosynthesis of deoxy-thymidine diphosphate-I-rhamnose, a naturally occurring deoxy-hexose (non-digestible carbohydrate) with strong propionigenic potential, as previously shown in RCC patients treated with a probiotic C. butyricum that boosted the efficacy of ICI (Dizman et al. 2022). Analyzing the pathway composition of SIGs in common among discovery and validation cohorts (FIG. 10C-E), we found that SIG1 harbored 183 pathways mostly related to dismal prognosis in other studies (such as CMP-legionaminate biosynthesis reported for CAR-T cells (Smith et al. 2022), or the superpathway of thiamine diphosphate biosynthesis, L-Histidine degradation, superpathway of L-Lysine, L-Threonine and L-Methionine biosynthesis I, or pyridoxal 5-phosphate salvage II (plants), already reported in advanced (as opposed to localized) breast cancers (Terrisse et al. 2021), while SIG2 retained the metabolic potential of mannan degradation and sulfur oxidation which remain of unknown significance between overt proinflammatory effects and elimination of SIG1-associated lactic acid bacteria (not shown).

### Example 6: Development of a User-Friendly PCR-Based TOPOSCORE Assay

Lastly, to transform the TOPOSCORE into a clinically actionable diagnosis tool, we contemplated i) circumventing the costly, laborious and time-consuming method of shotgun metagenomics by means of a PCR-based assay that can be performed within 48 hours for determining bacteria prevalence and ii) to restrain the numbers of SIG1- and SIG2-associated bacteria to expedite the assay. Based on the most prevalent (FIG. 11) MGS species found for the gut oncoimmunological fingerprints across various studies (Park et al. 2022) as well as probing set design feasibility and specificity, we focused our PCR-based assay on 16 SIG2- and 7 SIG1-associated bacteria in addition to Akkermansia spp. (FIG. 12). First, we attempted to re-run the TOPOSCORE calculation for the discovery cohort based on the MGS-based relative abundances of these 24 (instead of the full set of 75 bacteria). The Cox regression analysis of the impact of the “restricted 24-based TOPOSCORE” on the OS of the NSCLC cohort (n=393 patients) confirmed that patients with a TOPOSCORE falling within the SIG2+ Grey Akk^{L} region (1<FRnormCOUNT<2.065) exhibited a significantly prolonged clinical benefit to PD-1 blockade compared with individuals harboring a TOPOSCORE within SIG1+ Grey Akk^{0/H} region (FIG. 8C-D). Secondly, Spearman correlation indexes between MGS relative abundance (and therefore prevalence) and PCR amplification for each of the 24 bacterium-specific DNA validated the strategy of switching from MGS to PCR (FIG. 12, FIG. 8E). Thirdly, when utilizing the 24 bacteria-specific probe sets on the remaining fecal DNA (originally extracted for MGS) from the whole NSCLC cohort (n=313), we re-demonstrated and confirmed that OS was superior in those patients harboring a PCR-based TOPOSCORE within the SIG2+ Grey Akk^{L} region (1<FRnormCOUNT<2.065) (FIG. 8E).

In conclusion, we demonstrated that the development of a quick test, easily translated into clinical routine, is feasible but requires a prospective validation at this stage, on an independent cohort.

### Example 7: Development of a Further User-Friendly PCR-Based TOPOSCORE Assay

SIG2 species were selected after a random forest classifier within Sci-Kit learn package v1.0.1, having L1 regularization, with the following parameters: clf=RandomForestClassifier (n_estimators=est, class_weight=‘balanced_subsample’, random_state=0, oob_score=True, n_jobs=−1, max_depth=2, bootstrap=True, criterion=‘gini’). The 536 microbial species retrieved by the aforementioned RF model built on the TOPOSCORE within the discovery cohort (n=245) were hierarchically ordered following their descending Variable Feature Importance (VFI), and only microbial species with a VIF value higher than 1 were selected.

TABLE 11

| | Variable Feature |
|---|---|
| | Importance (VFI) - |
| | RF model |
| Group | TOPOSCORE-based |
| Clostridiales_{—}bacterium_{—}KLE1615 | 6.569 |
| Faecalibacterium_SGB15346 | 3.6691 |
| Clostridium_symbiosum | 3.5657 |
| Oscillibacter_sp_ER4 | 3.3654 |
| Enterocloster_{—}bolteae | 2.6583 |
| Agathobaculum_{—}butyriciproducens | 2.5388 |
| Eubacterium_{—}rectale | 2.4302 |
| Ruminococcus_gnavus | 2.3828 |
| Faecalibacillus_{—}intestinalis | 2.3119 |
| Dorea_{—}formicigenerans | 2.1616 |
| Lachnospira_{—}eligens | 2.0495 |
| Clostridium_sp_AM49_4BH | 2.0183 |
| Anaerobutyricum_{—}hallii | 1.9685 |
| Clostridium_sp_AF34_10BH | 1.9318 |
| Clostridium_fessum | 1.9288 |
| Lacrimispora_amygdalina | 1.8383 |
| Candidatus_{—}Cibiobacter_{—}qucibialis | 1.7393 |
| Ruminococcaceae_unclassified_SGB15236 | 1.5217 |
| Firmicutes_bacterium_AF16_15 | 1.4816 |
| Clostridium_sp_AM22_11AC | 1.4617 |
| Faecalibacterium_{—}prausnitzii | 1.4314 |
| Lachnospiraceae_bacterium_OM04_12BH | 1.4255 |
| Erysipelatoclostridium_{—}ramosum | 1.2413 |
| Blautia_{—}massiliensis | 1.2206 |
| Streptococcus_{—}parasanguinis | 1.1557 |
| Parasutterella_{—}excrementihominis | 1.13 |
| Dorea_{—}longicatena | 1.125 |
| Akkermansia_muciniphila | 1.1059 |
| Eubacterium_{—}ventriosum | 1.0799 |
| Coprococcus_comes | 1.0213 |

Microbial species selected by RF model applied on TOPOSCORE. Among the 536 microbial species 30 were selected based on their descending order of VIF. In bold the species falling within SIG1, while in italics the species falling within SIG2. Underlined are the species belonging to the selected 24-based species PCR assay. In order to ease such an assay, the selected species within SIG2 are reduced to seven: Eubacterium_rectale, Dorea_formicigenerans, Lachnospira_eligens, Faecalibacterium_prausnitzii, Parasutterella_excrementihominis, Dorea_longicatena, Eubacterium_ventriosum.

A TOPOSCORE_7_7 (seven members of SIG1 and seven members of SIG2) was calculated with:
SIG1_7=‘Enterocloster_bolteae’, ‘Eggerthella_lenta’, ‘Erysipelatoclostridiu m_ramosum’, ‘Haemophilus parainfluenzae’, ‘Dialister invisus’, ‘Veillonella atypica’, ‘Ent erococcus_faecalis’
SIG2_7=‘Eubacterium_rectale’, ‘Dorea_formicigenerans’, ‘Lachnospira_eli gens’, ‘Faecalibacterium prausnitzii’, ‘Parasutterella_excrementihominis’, ‘Dorea_longicat ena’, ‘Eubacterium_ventriosum’

As shown in table 12 below, this TOPOSCORE is still predictive.

TABLE 12
Coverage = 1.00

| | | | | | | | | | | PREDICTION | |
|---|---|---|---|---|---|---|---|---|---|---|---|
| | | INDIVIDUAL_PREDICTION | | | | | | PREDICTION | | NCoverage | |
| VALIDATION | | | Correct | Correct | | Uncorrect | Uncorrect | Correct | Uncorrect | Correct | Uncorrect |
| (L148) | Hits/total | Correct | NR | R | Uncorrect | NR | R | (%) | (%) | (%) | (%) |
| TOPOSCORE_7_7 | 108/108 | 66 | 32 | 34 | 42 | 24 | 18 | 61.1 | 38.9 | 61.1 | 38.9 |
| TOPOSCORE | 108/108 | 68 | 25 | 43 | 40 | 25 | 15 | 63.0 | 37.0 | 63.0 | 37.0 |

### DISCUSSION

Cross-cohort microbiome-trained machine learning consistently predicted outcomes of PD-(L)1 therapy despite heterogeneity between cohorts across geographical distribution but failed to reproducibly identify a gut fingerprint that robustly predicts clinical outcome on an individual basis. Three meta-analyses applying uniform computational approaches across different cancer types and therapies have not explained discrepancies among published cohorts (Gharaibeh et Jobin 2019; Limeta et al. 2020; Shaikh et al. 2021). Focusing on melanoma, two recent meta-analyses exploiting MGS data bases and using machine learning methodology did not entirely converge on the final “microbiotypes” associated with responses or resistance to immunotherapy (Lee et al. 2022; McCulloch et al. 2022).

In the present study, we readdressed this question using a different strategy. The emerging challenge of contemporary oncology is to reconstruct ecosystem networks and detect patterns of microbial species or communities leading to user-friendly diagnosis tools predicting the individual risk of immune resistance. As already discussed, baseline microbiota composition was optimally associated with clinical outcome when considering OS at 1 year after initiation of treatment (McCulloch et al. 2022; Heng et al. 2009). This reconstruction, which gave close to similar co-abundance networks using PEARSON or SPRING algorithms, was based on the assumption that SIG assembled a cooperative ecosystem of functionally related bacteria/archaea located at opposite ends and matching with clinical benefit and resistance respectively. Such opposite SIGs (SIG1 and SIG2) should represent suitable surrogates of the holo-ecosystem, considering the ratio of prevalence of each SIG member rather than a single or a couple of MGS species or genera of interest for each person. This rationale better handles the inherent heterogeneity among individuals, having in mind that the prevalence of each SIG1 member is lower (around 60% of SIG1 MGS have a prevalence <15%) than that of each SIG2 member (around 60% of SIG2 MGS have a prevalence >60%), in HV and cancer patients (FIG. 11). In fact, SIG1 contained 40 species belonging to the Enterocloster genus, and Streptococcacae and Veillonellaceae families as well as the Lactobacillales order (Enterococcaceae+Lactobacillaceae families) already identified in immune resistant patients (Spencer et al. 2021; Lee et al. 2022; McCulloch et al. 2022; Tsay et al. 2020; Yonekura et al. 2021). Interestingly, the Gut Microbiome Health Index (GMHI), an index proposed to predict the disease likelihood of an individual based on its fecal microbial composition (Gupta et al. 2020) found similar species in the non-healthy group to the ones here retrieved in our SIG1, and none of the species falling within the healthy group were retrieved in our SIG2, mainly because NSCLC patients are already diseased. Instead, SIG2 was composed of 34 species gathering Lachnospiraceae and Oscillospiraceae family members largely reported for their strong association with favorable clinical responses (Gopalakrishnan et al. 2018; Spencer et al. 2021; McCulloch et al. 2022; Yonekura et al. 2021; Frankel et al. 2017; Chaput et al. 2017). Computing the SIG1/SIG2 FRnormCOUNT on the 393 NSCLC patients, we found that 70% of individuals classified within SIG1+ were NR while 63% falling into SIG2+ were R. For individuals within the intermediate category (0.37<FRnormCOUNT<1.047), the relative abundance of Akkermansia spp., Verrucomicrobiaceae family member not statistically retained in the Pearson network model, efficiently complemented the FRnormCOUNT, allowing identification of NR in 59% and 84% cases of Akk⁰ and Akk^{H} (>4.8%) respectively, while classifying R in 59% cases of Akk^{L} individuals. The robustness of the TOPOSCORE could be extended to another cohort of advanced patients amenable to ICI (including 61 prospective cases of chemotherapy naïve NSCLC and 83 RCC). Extrapolating the calculations to our discovery and validation cohorts gathering 454 NSCLC patients, the TOPOSCORE Sensibility, Specificity, PPV, and NPV were 76.3%, 48%, 62.2% and 64.6%, respectively. Of note, the alternative state-of-the-art machine learning algorithms (including the SIRUS (Bénard et al. 2021)) and the Random Forest model ((«Random Forests|SpringerLink»s. d.)) did not perform as well. It is interesting to outline that 7.8% of HV fell into the SIG1+ category, suggesting that the TOPOSCORE could subserve the utility to eliminate distinct fecal transplantation donors. In contrast, the 4.5% of healthy individuals with MG profile harboring a FRnormCOUNT at zero (optimal TOPOSCORE) should be preferentially selected for FMT.

The Lactobacillales order was heavily represented within SIG1+, with 19/40 spp. including Enterococcaceae, Lactobacillaceae and Streptococcacae family members. Together with Veillonellaceae representatives (V. atypica, V. dispar, V. parvula, V. rogosae), they comprise many microbial components of the oral cavity, that can transit from the supraglottic compartment down to the bronchoalveolar space or the small intestine, as a result of pH fluctuations and/or co-medications (proton pump inhibitors) or dysphagia (Lee et al. 2022; Tsay et al. 2020; Cortellini et al. 2020; Imhann et al. 2016; Jackson et al. 2018). Oralization of the intestinal microbiota has been linked to failure of immunotherapy and immune-related adverse events (McCulloch et al. 2022). Many oral commensals suppress epithelial cell inflammatory responses by dampening PRR through Toll-like receptor (TLR) or NOD-like receptor (NLR) expression and signaling, while others suppress inflammatory responses by inhibiting NF-kB or releasing immunosuppressive anti-inflammatory cytokines, such as IL-10 (Cosseau et al. 2008; Bernardo et al. 2012; Santos Rocha et al. 2012). For instance, commensal Lactobacilli spp. with tryptophanase activity generates indole derivatives that can function as aryl hydrocarbon (AhR) ligands promoting the expansion of anti-inflammatory CD4+ Foxp3+ regulatory T cells (Treg) (Zelante et al. 2013). Double-stranded RNA from intestinal lactic acid bacteria induces interferon-p production by dendritic cells via TLR3 activation, thereby dampening inflammation (Kawashima et al. 2013). Veillonella spp. have the capacity to prime or expand TH17 pro-angiogenic and -oncogenic lymphocytes, that contribute to dismal prognosis and resistance to cytotoxicants in NSCLC (Tsay et al. 2020). The Enterocloster gen. (E. aldensis, E. asparagiformis, E. bolteae) represents a vancomycin-sensitive clade of immunosuppressive bacteria, dominant in the intestinal microbiota of people and patients suffering from aging and chronic inflammatory disorders including cancer patients (Limeta et al. 2020; Ghosh et al. 2020). By causing a beta-adrenergic receptor-dependent stress ileopathy and an Enterocloster genus-dominated dysbiosis, some malignancies (and other pathological disorders such as stroke) may increase gut permeability, favoring translocation of inflammatory mediators and bacteremia with immunosuppressive potential (Stanley et al. 2016; Yonekura et al. 2021). Hence, resistance to ICI appears to be driven by the over-representation of harmful bacteria more than by the absence of favorable MGS species, when considering the relative weight of SIG1+(or Akk^{H}) versus SIG2+ (or Akk⁰ or Akk^{L}) in the performance of the TOPOSCORE. This conclusion was also reached by McCulloch et al. finding that unfavorable “microbiotypes” composed of Gram negative Proteobacteria influenced the peripheral inflammatory tonus, the neutrophil-to-lymphocyte ratio and enterocyte exfoliation, paving the way to resistance to PD-1 blockade (McCulloch et al. 2022).

We also showed that it was possible to obtain comparable results using a PCR rather than an MGS-based TOPOSCORE, leveraging this diagnosis test within the routine tool box. In most cases, a good correlation was obtained between the two methods for the specific microorganism of interest. One exception was Faecalibacterium prausnitzii. The PCR assay used in the calculations was developed several years ago, and as more information about the heterogeneity of this species has merged, there is a clear need for further improvement of this particular PCR assay. Implementation of additional targets within SIG1 and SIG2 for probe set designing could further improve the diagnostic potential of such a PCR-based-TOPOSCORE assay.

The TOPOSCORE offers the unmet medical need of patient stratification based on “gut dysbiosis”, in order to ascribe resistance to ICI to an objective deviation from the “healthy” taxonomic composition (rather than to a cell-intrinsic molecular cue) and to guide the outcome of microbiota-centered interventions, for instance following switch from SIG1+ towards SIG2+ on an individual basis, at least in advanced lung and kidney cancer patients. More data and patient incrementation in trials are needed to design a TOPOSCORE in other malignancies (such as melanoma).

### Example 8: A SIG2+ Gut Microbiota Signature at Baseline is Associated with a Better Response to CAR T Cell Therapy

We prospectively and longitudinally collected fecal material, from patients receiving commercial CD19 CAR-T cells, at different time-points (Oncobiotics (Discovery of Microbiome-based Biomarkers for Patients With Cancer Using Metagenomic Approach); Sponsors: Gustave Roussy, Cancer Campus, Grand Paris; Sponsor Protocol N: CSET 2017/2619, ID-RCB N: 2017-A02010-53)): at baseline before lymphodepletive chemotherapy, between 7 and 15 days after CAR-T cell infusion, and 3 months after CAR-T cell infusion. Shotgun metagenomic analyses were performed on the patient'≤fecal samples, aiming at correlating the composition of the gut microbiota with response to CAR-T cells therapy.

Patients characteristics are described in Table 13. As expected, most of the patients had diffuse large B cell lymphoma and received axi-cel (CD28 co-stimulatory domain). The overall response rate observed (48.7%) was concordant with the ones observed in clinical trials.

TABLE 13
Patients characteristics

| | | n = 37 | |
|---|---|---|---|
| Age (median, quartile) | | 61.4 | [53.5-66.2] |
| Sex, male (%) | | 24 | 64.9% |
| Histologic subtype | Diffuse large B-cell lymphoma | 30 | 81.1% |
| | Follicular lymphoma | 3 | 8.1% |
| | B cell acute lymphoblastic leukemia | 2 | 5.4% |
| | Mantle cell lymphoma | 2 | 5.4% |
| Type of CAR T cells | axi-cel (CD28) | 30 | 81.1% |
| | brexu-cel (CD8) | 3 | 8.1% |
| | tisa-cel (4-1BB) | 4 | 10.8% |
| Response at 6 months | Response | 18 | 48.7% |
| | Progression | 10 | 27.0% |
| | Not yet known | 9 | 24.3% |
| Toxicity | Cytokine Release Syndrome | 34 | 91.9% |
| | Neurotoxicity | 17 | 45.9% |
| | Late onset hematological cytopenia | 13 | 35.1% |

Metagenomic sequencing were performed for 41 patients (data are still being collected) and analyses were obtained so far for 22 patients. Strikingly, we observed an absence of Akkermansia muciniphila in the fecal material from most of our B cell lymphoma cohort (90.9%).

The patient'≤TOPOSCORE was monitored using the metagenomic-based TOPOSCORE assay described in Example 2.

As shown in FIG. 13, the TOPOSCORE at baseline predicts overall response rate (ORR) and progression-free survival (PFS) in this cohort (p=0.0509).

### Example 9: Co-Abundance Networks within the Microbial Ecosystem of NSCLC Patients and Novel TOPOSCORE Calculation

Here we used prevalence and/or relative abundances of metagenomics species (MGS) to assess their cooperative potential within large species interacting groups (SIG) and the clinical relevance of SIGs for the response to PD1 blockade in the discovery cohort.

### Building Up Intestinal Communities (Species Interacting Groups)

Each MGS was categorized as either “low” or “high” based on the median of its relative abundance in the whole population of 245 subjects (≤median) or >median respectively). For those MGS which had a majority of null abundances (i.e., median=0), the MGS were categorized as “present” or “absent” (relative abundance>0 or =0 respectively). Cox Proportional Hazard (CoxPH) models were run to select MGS associated with the clinical variable “Overall Survival” with a Hazard Ratio (HR)<0.80 or ≥1.25 respectively. The purpose of this selection was to discard MGS with HR close to 1, which are unlikely to participate in the robustness of the signature. Among the 536 MGS identified by the shot gun MG of the discovery cohort, a total of 266 MGS was retained in the model (Table 14). The Akkermansia muciniphila MGS was not considered in this screening because its distribution was trichotomic with no linear dose-effect relationship with patient prognosis (Lisa Derosa et al. 2022b). Each pair of these 266 MGS was then analyzed by a Fisher'≤exact test on 2×2 contingency tables based on their absence/presence co-occurrences and scored by the by −log₁₀(p)×sign(OR−1) metrics, where p is the Fisher p-value and OR, the Odds Ratio of the 2×2 table. This metrics defined a score proportional to the significance of the interaction between two MGS (−log₁₀(p)) that is negative in case of co-exclusion pattern (OR<1) or positive in case of co-occurrence (OR>1). Interactions with a Bonferroni-corrected p-value≤0.05 were retained for analysis. A total of 180 connected MGS were then clustered with Ward'≤method and Manhattan distance resulting in the identification of 7 clusters (C1 to C7) (Table 14). Two clusters (C5 and C6) contained 37 MGS mostly (95%) associated with OS<12 (HR 1.25) that were used to define the SIG1 signature. Three clusters (C1, C2, C3) contained 45 MGS all associated with OS>12 months (HR≤0.80) that were used to define the SIG2 signature (Table 14). All the other clusters failed to correlate with OS. In addition, interactions within SIG1 and SIG2 MGS were 99% and 100% positive respectively (co-occurrence patterns), while edges in-between SIG1 and SIG2 MGS were 98% negative (co-exclusion patterns), thus reflecting a significant and opposite topological separation (data not shown). These results are supported by the fact that SIG1 contained members belonging to the Enterocloster genus, and Streptococcaceae, Veillonellaceae and Lactobacillaceae families that were already associated with dismal prognosis and immunoresistance (Yonekura et al. 2021; Spencer et al. 2021; Lee et al. 2022; McCulloch et al. 2022; Tsay et al. 2020). Conversely, SIG2 contained Lachnospiraceae (species from the genus Blautia, Roseburia, Dorea, Eubacterium), and Oscillospiraceae family members (Faecalibacterium prausnitzii, Ruminococcus bicirculans and R. lactaris), which were found associated with general health and favorable clinical responses to ICI (Gopalakrishnan et al. 2018; Messaoudene et al. 2022).

TABLE 14
List of the 266 MGS retained in the CoxPH model, of the 180 selected
in each of the seven clusters and of 82 selected in SIG 1 or 2

| | | | CoxPH | | |
|---|---|---|---|---|---|
| MGS | HR | HR95% CI | p-value | CLUSTER | SIG |
| Acidaminococcusfermentans | 0.58 | 0.28-1.18 | 0.1331 | C4 | |
| ActinomycesSGB17154 | 1.96 | 0.86-4.44 | 0.1079 | | |
| Actinomycesgraevenitzii | 4.69 | 2.34-9.41 | 0.0000 | C6 | SIG1 |
| Agathobaculumbutyriciproducens | 0.75 | 0.55-1.01 | 0.0618 | C2 | SIG2 |
| Alistipesdispar | 0.77 | 0.54-1.08 | 0.1284 | C4 | |
| Alistipesmegaguti | 1.67 | 0.74-3.79 | 0.2202 | | |
| Alistipesprovencensis | 0.79 | 0.29-2.14 | 0.6476 | | |
| Alistipesputredinis | 0.75 | 0.55-1.03 | 0.0733 | C4 | |
| Alistipessenegalensis | 0.78 | 0.57-1.08 | 0.1366 | C4 | |
| AlistipesspAF1716 | 1.26 | 0.82-1.93 | 0.2934 | | |
| Alistipestimonensis | 0.75 | 0.44-1.28 | 0.2957 | | |
| Alloscardoviaomnicolens | 1.49 | 0.86-2.59 | 0.1557 | C6 | SIG1 |
| Alphaproteobacteriabacterium | 0.75 | 0.43-1.29 | 0.2977 | | |
| Amedibacillusdolichus | 0.67 | 0.34-1.30 | 0.2351 | | |
| Amedibacteriumintestinale | 1.27 | 0.62-2.58 | 0.5153 | | |
| Anaerobutyricumhallii | 0.62 | 0.46-0.85 | 0.0027 | C3 | SIG2 |
| Anaerostipescaccae | 1.33 | 0.90-1.98 | 0.1569 | C5 | SIG1 |
| Anaerostipeshadrus | 0.78 | 0.57-1.06 | 0.1101 | C1 | SIG2 |
| Anaerotignumfaecicola | 0.8 | 0.57-1.11 | 0.1778 | C3 | SIG2 |
| Anaerotruncusmassiliensis | 1.3 | 0.92-1.83 | 0.1426 | C4 | |
| Bacillibacterium | 1.37 | 0.92-2.05 | 0.1207 | C4 | |
| BacilliunclassifiedSGB6422 | 1.6 | 0.75-3.44 | 0.2261 | C4 | |
| BacilliunclassifiedSGB6428 | 1.76 | 0.92-3.34 | 0.0858 | C4 | |
| BacilliunclassifiedSGB6473 | 0.79 | 0.39-1.61 | 0.5192 | C4 | |
| BacilliunclassifiedSGB6571 | 1.36 | 0.85-2.15 | 0.1959 | C4 | |
| Bacteroidaceaebacterium | 0.79 | 0.56-1.11 | 0.1743 | C4 | |
| Bacteroidescongonensis | 0.79 | 0.37-1.69 | 0.5415 | | |
| Bacteroidesfaecis | 0.79 | 0.51-1.23 | 0.2993 | | |
| Bacteroidesfinegoldii | 0.71 | 0.51-0.99 | 0.0453 | C4 | |
| Bacteroidesfragilis | 1.48 | 1.08-2.01 | 0.0137 | C7 | |
| Bacteroidesndongoniae | 0.77 | 0.42-1.43 | 0.4141 | C4 | |
| Bacteroidessalyersiae | 1.31 | 0.95-1.83 | 0.1038 | C4 | |
| Bacteroidesthetaiotaomicron | 1.44 | 1.06-1.96 | 0.0200 | C4 | |
| Bacteroidestogonis | 0.72 | 0.42-1.23 | 0.2348 | | |
| Bifidobacteriumadolescentis | 0.68 | 0.48-0.95 | 0.0260 | C4 | |
| Bifidobacteriumanimalis | 1.38 | 0.80-2.39 | 0.2504 | | |
| Bifidobacteriumbifidum | 0.79 | 0.52-1.19 | 0.2631 | C7 | |
| Bifidobacteriumbreve | 1.41 | 0.72-2.77 | 0.3174 | | |
| Bifidobacteriumdentium | 1.47 | 1.07-2.03 | 0.0164 | C6 | SIG1 |
| Bifidobacteriumpullorum | 0.75 | 0.38-1.49 | 0.4146 | | |
| Bilophilawadsworthia | 1.47 | 1.08-2.00 | 0.0149 | C4 | |
| Bittarellamassiliensis | 0.77 | 0.55-1.09 | 0.1376 | C7 | |
| BlautiaSGB4805 | 1.4 | 0.98-2.00 | 0.0657 | C4 | |
| Blautiahydrogenotrophica | 0.69 | 0.41-1.18 | 0.1747 | | |
| Blautiamassiliensis | 0.69 | 0.50-0.94 | 0.0173 | C3 | SIG2 |
| Blautiaproducta | 1.3 | 0.80-2.10 | 0.2895 | C5 | SIG1 |
| Blautiaschinkii | 2.46 | 1.08-5.60 | 0.0319 | | |
| BlautiaspAF1910LB | 0.55 | 0.28-1.07 | 0.0779 | | |
| BlautiaspMSK211 | 0.51 | 0.24-1.08 | 0.0789 | | |
| BlautiaspOF0315BH | 0.52 | 0.23-1.18 | 0.1181 | | |
| Blautiastercoris | 0.7 | 0.40-1.23 | 0.2128 | C4 | |
| Blautiawexlerae | 0.73 | 0.54-0.99 | 0.0457 | C3 | SIG2 |
| Brachyspiraaalborgi | 0.62 | 0.23-1.67 | 0.3408 | | |
| ButyricicoccusSGB14990 | 0.33 | 0.10-1.02 | 0.0542 | C4 | |
| ButyricicoccusspAM2923AC | 0.58 | 0.26-1.31 | 0.1914 | | |
| Butyricimonasfaecihominis | 0.8 | 0.56-1.13 | 0.2061 | | |
| Campylobacterconcisus | 1.91 | 0.84-4.33 | 0.1215 | C6 | SIG1 |
| Campylobactergracilis | 1.83 | 0.96-3.48 | 0.0664 | C6 | SIG1 |
| CandidatusAllochristensenellacaecavium | 0.79 | 0.56-1.12 | 0.1871 | C7 | |
| CandidatusAlloruminococcusvanvlietii | 0.72 | 0.43-1.21 | 0.2122 | | |
| CandidatusAristotellaavistercoris | 1.63 | 1.13-2.35 | 0.0086 | C7 | |
| CandidatusAvimonasnarfia | 0.71 | 0.50-1.01 | 0.0559 | C7 | |
| CandidatusAvispirillumfaecium | 1.53 | 0.93-2.53 | 0.0964 | C4 | |
| CandidatusCibiobacterqucibialis | 0.72 | 0.53-0.97 | 0.0336 | C2 | SIG2 |
| CandidatusGallimonasintestinalis | 1.31 | 0.73-2.36 | 0.3661 | | |
| CandidatusGallimonasmerdae | 0.67 | 0.30-1.52 | 0.3388 | C4 | |
| CandidatusHeritagellagallinarum | 1.6 | 0.79-3.26 | 0.1954 | | |
| CandidatusHeritagellaintestinalis | 0.73 | 0.41-1.30 | 0.2867 | | |
| CandidatusHeteroclostridiumcaecigallinarum | 0.67 | 0.35-1.28 | 0.2253 | C4 | |
| CandidatusHeteroruminococcusfaecigallinarum | 1.64 | 0.99-2.71 | 0.0546 | C7 | |
| CandidatusMetalachnospiragallinarum | 1.55 | 0.92-2.60 | 0.0992 | C7 | |
| CandidatusMetaruminococcusgallistercoris | 0.78 | 0.49-1.23 | 0.2764 | C7 | |
| CandidatusNeochristensenellagallicola | 0.77 | 0.49-1.21 | 0.2594 | C7 | |
| CandidatusNeoclostridiumroslinense | 1.28 | 0.72-2.26 | 0.3967 | C4 | |
| CandidatusParalachnospiracaecorum | 1.59 | 1.01-2.50 | 0.0464 | C7 | |
| CandidatusPseudolachnospiraavium | 0.79 | 0.50-1.26 | 0.3270 | C7 | |
| CandidatusPseudoscilispirafalkowii | 1.46 | 0.74-2.87 | 0.2713 | | |
| Catabacterhongkongensis | 1.47 | 1.01-2.15 | 0.0426 | C7 | |
| Christensenellatimonensis | 1.75 | 0.82-3.74 | 0.1492 | | |
| Christensenellaceaebacterium | 0.73 | 0.50-1.08 | 0.1146 | | |
| ChristensenellaceaebacteriumNSJ44 | 1.33 | 0.79-2.23 | 0.2791 | C7 | |
| Cloacibacillusevryensis | 1.54 | 0.76-3.14 | 0.2328 | | |
| ClostridiabacteriumUC511D1 | 0.69 | 0.51-0.94 | 0.0183 | C7 | |
| ClostridiaunclassifiedSGB14844 | 0.8 | 0.59-1.09 | 0.1502 | C7 | |
| ClostridiaunclassifiedSGB15402 | 1.8 | 1.23-2.63 | 0.0026 | C7 | |
| ClostridiaunclassifiedSGB3983 | 1.26 | 0.74-2.14 | 0.4004 | C4 | |
| ClostridiaunclassifiedSGB4367 | 0.79 | 0.54-1.16 | 0.2297 | C4 | |
| ClostridiaunclassifiedSGB6317 | 0.71 | 0.40-1.25 | 0.2349 | C4 | |
| ClostridiaunclassifiedSGB6344 | 1.26 | 0.56-2.86 | 0.5792 | | |
| ClostridiaunclassifiedSGB6385 | 0.72 | 0.30-1.76 | 0.4762 | C4 | |
| ClostridiaunclassifiedSGB71368 | 0.62 | 0.30-1.26 | 0.1887 | | |
| Clostridiaceaebacterium | 0.77 | 0.56-1.04 | 0.0911 | C3 | SIG2 |
| ClostridiaceaebacteriumNSJ31 | 0.65 | 0.30-1.39 | 0.2658 | | |
| ClostridiaceaebacteriumNSJ33 | 2.49 | 1.02-6.10 | 0.0452 | | |
| ClostridiaceaebacteriumOM086BH | 0.76 | 0.56-1.03 | 0.0732 | C2 | SIG2 |
| ClostridiaceaeunclassifiedSGB4769 | 0.75 | 0.54-1.06 | 0.1019 | C2 | SIG2 |
| ClostridialesFamilyXIIIbacteriumRF744FATWT3 | 0.61 | 0.32-1.16 | 0.1343 | C4 | |
| Clostridialesbacterium | 0.78 | 0.57-1.06 | 0.1135 | C7 | |
| Clostridialesbacterium1747FAA | 1.29 | 0.86-1.93 | 0.2138 | | |
| ClostridialesbacteriumBX7 | 0.78 | 0.48-1.24 | 0.2914 | C4 | |
| ClostridialesbacteriumKLE1615 | 0.57 | 0.42-0.78 | 0.0004 | C1 | SIG2 |
| ClostridialesbacteriumNSJ40 | 1.51 | 1.03-2.20 | 0.0325 | C4 | |
| ClostridialesbacteriumUBA1390 | 0.75 | 0.54-1.04 | 0.0837 | C4 | |
| ClostridialesunclassifiedSGB15145 | 0.75 | 0.53-1.04 | 0.0876 | C2 | SIG2 |
| ClostridiumSGB6179 | 0.69 | 0.43-1.12 | 0.1336 | C4 | |
| Clostridiumdisporicum | 1.31 | 0.71-2.42 | 0.3880 | C4 | |
| Clostridiumfessum | 0.76 | 0.56-1.03 | 0.0743 | C2 | SIG2 |
| Clostridiumhylemonae | 1.25 | 0.61-2.55 | 0.5380 | C7 | |
| Clostridiuminnocuum | 1.36 | 1.00-1.85 | 0.0471 | C5 | SIG1 |
| Clostridiumperfringens | 1.63 | 0.94-2.82 | 0.0814 | C6 | SIG1 |
| Clostridiumphoceensis | 0.72 | 0.53-0.99 | 0.0398 | C4 | |
| Clostridiumsaccharogumia | 1.63 | 1.04-2.55 | 0.0336 | | |
| Clostridiumscindens | 1.5 | 1.09-2.06 | 0.0128 | C5 | SIG1 |
| ClostridiumspAF3410BH | 0.66 | 0.48-0.90 | 0.0079 | C1 | SIG2 |
| ClostridiumspAM2211AC | 0.75 | 0.55-1.03 | 0.0751 | C2 | SIG2 |
| ClostridiumspAM333 | 0.8 | 0.56-1.13 | 0.2091 | C2 | SIG2 |
| ClostridiumspAM494BH | 0.68 | 0.47-0.99 | 0.0423 | C3 | SIG2 |
| ClostridiumspAT4 | 0.69 | 0.44-1.08 | 0.1036 | C7 | |
| ClostridiumspMarseilleP3244 | 4.02 | 1.94-8.30 | 0.0002 | C7 | |
| Clostridiumspiroforme | 1.26 | 0.86-1.86 | 0.2403 | C7 | |
| Clostridiumsymbiosum | 1.47 | 1.08-2.00 | 0.0149 | C5 | SIG1 |
| CollinsellaSGB14754 | 1.34 | 0.84-2.15 | 0.2170 | C6 | SIG1 |
| Collinsellaaerofaciens | 0.69 | 0.50-0.94 | 0.0191 | C4 | |
| Collinsellamassiliensis | 1.26 | 0.73-2.19 | 0.4061 | | |
| Collinsellastercoris | 1.57 | 0.64-3.84 | 0.3206 | | |
| Coprobacterfastidiosus | 0.74 | 0.54-1.01 | 0.0602 | C3 | SIG2 |
| Coprococcuscomes | 0.74 | 0.54-1.00 | 0.0504 | C2 | SIG2 |
| Coprococcuseutactus | 0.67 | 0.47-0.94 | 0.0223 | C1 | SIG2 |
| DesulfovibriospPG178WT4 | 1.84 | 0.81-4.17 | 0.1450 | C7 | |
| DialisterSGB5820 | 0.51 | 0.21-1.25 | 0.1423 | | |
| Dialistersuccinatiphilus | 0.74 | 0.36-1.51 | 0.4131 | C4 | |
| Doreaformicigenerans | 0.71 | 0.52-0.97 | 0.0288 | C1 | SIG2 |
| Dorealongicatena | 0.8 | 0.59-1.09 | 0.1516 | C1 | SIG2 |
| DoreaspAF247LB | 1.37 | 0.72-2.60 | 0.3400 | | |
| Duodenibacillusmassiliensis | 0.13 | 0.02-0.91 | 0.0402 | | |
| Eggerthellaceaebacterium | 1.28 | 0.91-1.80 | 0.1536 | C4 | |
| EggerthellaceaeunclassifiedSGB14332 | 0.78 | 0.29-2.11 | 0.6293 | | |
| EggerthellaceaeunclassifiedSGB14341 | 0.77 | 0.52-1.15 | 0.2034 | | |
| Eisenbergiellatayi | 1.39 | 1.02-1.89 | 0.0370 | C7 | |
| Emergenciatimonensis | 1.51 | 0.87-2.61 | 0.1436 | C7 | |
| Enormamassiliensis | 1.4 | 0.96-2.04 | 0.0817 | C6 | SIG1 |
| Enterobacterhormaechei | 1.54 | 0.75-3.15 | 0.2362 | | |
| Enteroclosteraldensis | 1.75 | 1.28-2.38 | 0.0005 | C5 | SIG1 |
| Enteroclosterbolteae | 1.28 | 0.94-1.74 | 0.1193 | C5 | SIG1 |
| Enteroclosterclostridioformis | 1.64 | 1.20-2.24 | 0.0019 | C5 | SIG1 |
| Enterococcusdurans | 1.75 | 0.89-3.45 | 0.1060 | | |
| Enterococcusfaecalis | 1.73 | 1.12-2.67 | 0.0131 | C7 | |
| Erysipelatoclostridiumramosum | 1.44 | 1.06-1.96 | 0.0210 | C5 | SIG1 |
| Erysipelotrichaceaebacterium | 0.79 | 0.53-1.17 | 0.2438 | C4 | |
| Erysipelotrichaceaebacterium3153 | 0.76 | 0.40-1.45 | 0.4047 | | |
| Escherichiacoli | 1.46 | 1.07-1.99 | 0.0168 | | |
| EubacteriaceaebacteriumCHKCI004 | 1.43 | 0.81-2.52 | 0.2214 | C7 | |
| Eubacteriumcallanderi | 2.03 | 1.23-3.36 | 0.0059 | C7 | |
| Eubacteriumramulus | 0.74 | 0.53-1.04 | 0.0840 | C3 | SIG2 |
| Eubacteriumrectale | 0.64 | 0.47-0.87 | 0.0047 | C2 | SIG2 |
| EubacteriumspAn11 | 2.54 | 1.04-6.22 | 0.0412 | | |
| Eubacteriumventriosum | 0.58 | 0.42-0.80 | 0.0009 | C1 | SIG2 |
| Faecalibacillusfaecis | 0.65 | 0.35-1.19 | 0.1605 | C4 | |
| Faecalibacillusintestinalis | 0.7 | 0.51-0.96 | 0.0257 | C1 | SIG2 |
| FaecalibacteriumSGB15346 | 0.79 | 0.58-1.07 | 0.1237 | C2 | SIG2 |
| Faecalibacteriumprausnitzii | 0.69 | 0.51-0.94 | 0.0193 | C1 | SIG2 |
| Faecalicatenacontorta | 1.39 | 0.84-2.30 | 0.1986 | C7 | |
| Faecalitaleacylindroides | 0.71 | 0.50-1.03 | 0.0685 | C4 | |
| FirmicutesbacteriumAF1615 | 0.66 | 0.49-0.91 | 0.0097 | C2 | SIG2 |
| Fournierellamassiliensis | 0.79 | 0.40-1.54 | 0.4839 | C6 | SIG1 |
| Frisingicoccuscaecimuris | 1.35 | 0.66-2.76 | 0.4055 | | |
| Gemellasanguinis | 0.7 | 0.29-1.70 | 0.4297 | | |
| GemmigerSGB15295 | 1.35 | 0.86-2.10 | 0.1905 | C4 | |
| Gemmigerformicilis | 0.7 | 0.51-0.95 | 0.0231 | C2 | SIG2 |
| Granulicatellaadiacens | 1.3 | 0.57-2.94 | 0.5325 | C6 | SIG1 |
| Haemophilusparainfluenzae | 1.44 | 1.06-1.97 | 0.0204 | C4 | |
| Harryflintiaacetispora | 1.36 | 0.95-1.95 | 0.0938 | C7 | |
| HoldemaniaspMarseilleP2844 | 0.72 | 0.41-1.24 | 0.2335 | | |
| Hungatellahathewayi | 1.59 | 1.15-2.20 | 0.0047 | C5 | SIG1 |
| Hydrogeniiclostidiummannosilyticum | 0.66 | 0.47-0.92 | 0.0144 | C7 | |
| Intestinimonasmassiliensis | 2.19 | 1.48-3.24 | 0.0001 | C7 | |
| Klebsiellagrimontii | 1.55 | 0.84-2.86 | 0.1636 | | |
| LachnoclostridiumSGB4598 | 0.72 | 0.34-1.54 | 0.3992 | C7 | |
| Lachnospiraeligens | 0.71 | 0.53-0.97 | 0.0325 | C1 | SIG2 |
| Lachnospirapectinoschiza | 0.73 | 0.53-1.00 | 0.0526 | C3 | SIG2 |
| LachnospiraspNSJ43 | 0.74 | 0.49-1.09 | 0.1282 | C3 | SIG2 |
| LachnospiraceaebacteriumNSJ29 | 1.31 | 0.67-2.57 | 0.4294 | | |
| LachnospiraceaebacteriumNSJ46 | 0.8 | 0.35-1.81 | 0.5876 | | |
| LachnospiraceaebacteriumOM0412BH | 0.78 | 0.54-1.14 | 0.2052 | C3 | SIG2 |
| LachnospiraceaebacteriumUBA1818 | 1.36 | 0.56-3.32 | 0.4970 | | |
| LachnospiraceaebacteriumWCA3601WT6H | 0.71 | 0.53-0.97 | 0.0321 | C2 | SIG2 |
| LachnospiraceaeunclassifiedSGB4890 | 0.68 | 0.28-1.65 | 0.3909 | | |
| Lacrimisporaamygdalina | 0.74 | 0.54-1.01 | 0.0546 | C2 | SIG2 |
| Lacrimisporacelerecrescens | 0.79 | 0.54-1.15 | 0.2215 | C4 | |
| Lacticaseibacillusparacasei | 1.81 | 1.10-3.01 | 0.0207 | C6 | SIG1 |
| Lacticaseibacillusrhamnosus | 2.05 | 1.22-3.44 | 0.0069 | C7 | |
| Lactobacillusdelbrueckii | 1.72 | 0.91-3.28 | 0.0970 | | |
| Lactobacillusgasseri | 1.27 | 0.86-1.87 | 0.2283 | C6 | SIG1 |
| Lactobacillusvaginalis | 2.31 | 1.31-4.07 | 0.0039 | C6 | SIG1 |
| Lactococcuslaudensis | 1.5 | 0.62-3.67 | 0.3692 | | |
| Lactonifactorlongoviformis | 0.67 | 0.21-2.11 | 0.4970 | | |
| LentisphaeriaunclassifiedSGB9198 | 0.52 | 0.21-1.29 | 0.1602 | | |
| Ligilactobacillussalivarius | 1.57 | 0.95-2.60 | 0.0801 | C6 | SIG1 |
| Limosilactobacillusfermentum | 1.52 | 0.97-2.39 | 0.0685 | C6 | SIG1 |
| Limosilactobacillusoris | 1.28 | 0.63-2.62 | 0.4983 | C6 | SIG1 |
| MarvinbryantiaSGB4691 | 2.98 | 1.38-6.42 | 0.0053 | | |
| Massilicolitimonensis | 1.54 | 0.83-2.85 | 0.1673 | C7 | |
| Massilimicrobiotatimonensis | 1.4 | 0.81-2.43 | 0.2308 | C7 | |
| Mediterraneibacterbutyricigenes | 0.66 | 0.47-0.92 | 0.0150 | C3 | SIG2 |
| Mediterraneibacterspgm002 | 2.16 | 1.06-4.41 | 0.0350 | | |
| Megamonasfuniformis | 0.64 | 0.30-1.38 | 0.2562 | | |
| Megasphaeramicronuciformis | 2.06 | 1.19-3.57 | 0.0099 | C6 | SIG1 |
| MethanomassiliicoccaceaearchaeonDOK | 1.35 | 0.55-3.29 | 0.5154 | | |
| Mitsuokellajalaludinii | 0.7 | 0.39-1.27 | 0.2424 | C4 | |
| Mogibacteriumdiversum | 2.55 | 1.38-4.73 | 0.0029 | | |
| Neglectatimonensis | 1.37 | 0.86-2.19 | 0.1856 | C7 | |
| OlsenellaSGB14390 | 1.9 | 0.84-4.29 | 0.1250 | | |
| Olsenellatimonensis | 0.73 | 0.38-1.38 | 0.3333 | C4 | |
| Opitutalesbacterium | 1.33 | 0.88-2.02 | 0.1712 | C4 | |
| OscillibacterSGB15077 | 0.73 | 0.43-1.24 | 0.2444 | C7 | |
| OscillibacterspER4 | 0.78 | 0.57-1.07 | 0.1233 | C2 | SIG2 |
| Parabacteroidesgordonii | 1.47 | 0.72-2.99 | 0.2943 | | |
| Parabacteroidesjohnsonii | 0.8 | 0.54-1.16 | 0.2350 | | |
| Parabacteroidesmassiliensis | 0.75 | 0.33-1.71 | 0.4996 | | |
| Peptococcaceaebacterium | 0.76 | 0.50-1.15 | 0.1903 | C4 | |
| Phocaeicolacoprocola | 1.65 | 1.09-2.52 | 0.0190 | C4 | |
| Phocaeicolacoprophilus | 1.74 | 0.94-3.22 | 0.0802 | | |
| Phocaeicolamassiliensis | 0.77 | 0.55-1.06 | 0.1123 | C3 | SIG2 |
| PrevotellaSGB1680 | 0.72 | 0.27-1.93 | 0.5087 | C4 | |
| PrevotellacopricladeC | 0.34 | 0.13-0.93 | 0.0348 | C4 | |
| PrevotellaspP451 | 0.7 | 0.26-1.90 | 0.4893 | C4 | |
| Prevotellastercorea | 1.91 | 0.78-4.66 | 0.1563 | | |
| Proteusmirabilis | 1.56 | 0.69-3.54 | 0.2863 | C6 | SIG1 |
| Pseudoflavonifractorcapillosus | 0.8 | 0.57-1.11 | 0.1753 | C7 | |
| Pyramidobacterpiscolens | 1.69 | 0.83-3.46 | 0.1489 | | |
| Raoultibactermassiliensis | 1.41 | 0.62-3.18 | 0.4144 | | |
| RhodobacteraceaeunclassifiedSGB53807 | 3.22 | 1.57-6.61 | 0.0015 | | |
| Rikenellaceaebacterium | 0.77 | 0.47-1.26 | 0.3049 | C4 | |
| Roseburiahominis | 0.66 | 0.49-0.90 | 0.0089 | C1 | SIG2 |
| Roseburiaintestinalis | 0.76 | 0.56-1.03 | 0.0753 | C3 | SIG2 |
| Roseburiainulinivorans | 0.74 | 0.55-1.01 | 0.0592 | C1 | SIG2 |
| RoseburiaspAF0212 | 0.78 | 0.56-1.09 | 0.1466 | C3 | SIG2 |
| Rothiadentocariosa | 1.64 | 0.72-3.72 | 0.2358 | | |
| RuminococcaceaebacteriumD5 | 1.74 | 1.23-2.46 | 0.0018 | C4 | |
| RuminococcaceaebacteriumUBA1375 | 1.52 | 0.67-3.45 | 0.3117 | | |
| RuminococcaceaeunclassifiedSGB14835 | 1.3 | 0.64-2.66 | 0.4655 | C4 | |
| RuminococcaceaeunclassifiedSGB15309 | 0.61 | 0.34-1.10 | 0.1026 | C4 | |
| Ruminococcusbicirculans | 0.68 | 0.50-0.93 | 0.0154 | C3 | SIG2 |
| Ruminococcuscallidus | 0.62 | 0.40-0.96 | 0.0341 | C4 | |
| Ruminococcuslactaris | 0.77 | 0.56-1.06 | 0.1134 | C3 | SIG2 |
| Ruthenibacteriumlactatiformans | 1.42 | 1.04-1.93 | 0.0270 | C7 | |
| Scardoviawiggsiae | 1.74 | 1.12-2.70 | 0.0145 | | |
| Slackiaequolifaciens | 2.13 | 1.00-4.57 | 0.0512 | | |
| Slackiaisoflavoniconvertens | 0.77 | 0.51-1.15 | 0.2009 | C4 | |
| SolobacteriumSGB6833 | 1.42 | 0.81-2.51 | 0.2246 | | |
| Streptococcusanginosus | 1.99 | 1.33-2.98 | 0.0008 | C6 | SIG1 |
| Streptococcusgordonii | 1.79 | 1.04-3.10 | 0.0372 | C6 | SIG1 |
| Streptococcusmutans | 1.54 | 0.89-2.67 | 0.1248 | C6 | SIG1 |
| Streptococcusoralis | 0.79 | 0.29-2.13 | 0.6385 | C6 | SIG1 |
| Streptococcusparasanguinis | 1.48 | 1.09-2.02 | 0.0124 | C6 | SIG1 |
| Streptococcusrubneri | 0.59 | 0.22-1.60 | 0.2986 | | |
| Streptococcussalivarius | 1.3 | 0.95-1.77 | 0.0956 | C6 | SIG1 |
| Streptococcussanguinis | 1.74 | 1.05-2.88 | 0.0322 | | |
| SubdoligranulumSGB15305 | 1.43 | 0.77-2.67 | 0.2600 | | |
| Sutterellaseckii | 0.56 | 0.21-1.51 | 0.2508 | | |
| SutterellaspAM1139 | 1.3 | 0.78-2.14 | 0.3130 | | |
| Synergistaceaebacterium | 1.25 | 0.64-2.46 | 0.5123 | | |
| Traorellamassiliensis | 0.8 | 0.42-1.51 | 0.4873 | | |
| Veillonellaatypica | 1.28 | 0.91-1.80 | 0.1499 | C6 | SIG1 |
| Veillonelladispar | 1.42 | 1.03-1.96 | 0.0341 | C6 | SIG1 |
| Veillonellaparvula | 1.41 | 1.04-1.92 | 0.0274 | C6 | SIG1 |

### Scoring System for Each Individual

Next, we reduced the information of this whole-population-based network down to a unidimensional score. Each patient of the discovery cohort was then scored with a S score computed as the difference of proportions between present (relative abundance>0) SIG2 and SIG1 MGS and scaled from 0 to 1: S=(#S/G2/45−#S/G1/37+1)12. A score of 0 indicates that all MGS of the SIG1 signature have strictly positive relative abundances and all MGS of the SIG2 signature have null relative abundances. Conversely, a score of 1 indicates that all MGS of the SIG1 and SIG2 signature have null and strictly positive relative abundances respectively. The performance of this S score as predictor of OS12 was analyzed by a Receiver Operating Characteristic (ROC) analysis. Two S scores, 0.5351 and 0.7911, were identified as local maxima of the Youden index (Specificity+Specificity−1, FIG. 14), and were used as cut-off values to define three categories: SIG1 if S≤0.5351, SIG2 if S≥0.7911, and “gray zone” otherwise (FIG. 15A). Hence, 69% and 23% of patients falling into S scoring<0.5351 and S≥0.7911 presented an OS<12 months respectively (FIG. 15A, Table 15). By extension, the Cox regression analysis of the clinical impact of the S score on OS highlighted that patients with a S score≥0.7911 (called “SIG2” henceforth) exhibited a significantly prolonged clinical benefit to PD-1 blockade than patients falling into the Gray zone or a S score≤0.5351 (called “SIG1” henceforth) (FIG. 15B). About 22.5%, 31%, and 46.5% of patients in the discovery cohort fell into SIG2, SIG1 and Gray zone category respectively.

Next, we analyzed the intraindividual dynamics of the S score in 32 NSCLC patients who were sampled twice, before and within 3 months after treatment start. Interestingly, 33% and 25% of SIG2 and SIG1 joined the Gray zone respectively while half of patients classified in the Gray score shifted to SIG2 and no patients changed from Gray to SIG1 (FIG. 15C). Altogether, 67%, 50% and 70% of individuals within SIG2, Gray or SIG1 remained in the same category respectively (FIG. 15C). Responders at 3 months (n=12) resulted from SIG2 (n=8) and Grey (n=4) subject categories. Non responders (n=20) resulted from Grey (n=6) and SIG1 subjects (n=14) subject categories.

### Refining the Predictive Model

To solve the uncertainty of the “Gray zone” which represented about half of NSCLC patients, we segregated individuals according to the trichotomized distribution of Akkermansia muciniphila (Akk) relative abundance (FIG. 15D) (Lisa Derosa et al. 2022a). Normal levels of Akk (0<Akk≤4.799, Akk^{norm}) may be considered as a surrogate of host fitness in comparison with abnormal levels (Akk≥4.8, Akk^{high}) or the absence of Akk (Akk⁰)(Lisa Derosa et al. 2022a). Starting from here, Gray zone patients who harbored physiological Akk levels (about 19% of the whole cohort) were considered comparable to SIG2 subject category, while Gray zone patients devoid of (Akk⁰) or harboring high Akk levels and constituting about 23% and 4% of the whole cohort, respectively, were considered comparable to SIG1 subject category (Table 1). Ultimately, according to S scoring and Akk level, we built a final binomial categorical score of “immunoresistance-related” dysbiosis, named “TOPOSCORE”, to classify NSCLC patients into two risk categories, either SIG2+(comprising SIG2 [TOPOSCORE=1]+ Gray zone Akk^{norm} [TOPOSCORE=2]) or SIG1+(encompassing SIG1 [TOPOSCORE=5]+ Gray zone Akk^{high} [TOPOSCORE=4]+ Gray zone Akk⁰ [TOPOSCORE=3]) individuals (FIG. 15D, Table 15). Indeed, the Cox regression analysis of the clinical impact of the TOPOSCORE on progression-free and overall survivals highlighted that patients with a TOPOSCORE falling within “SIG2+” exhibited a significantly prolonged clinical benefit to PD-1 blockade (Table 15, FIG. 15E, left and right panels) compared with patients with a TOPOSCORE falling within “SIG1+”. Moreover, even after adjusting for renown risk factors (age, gender, body mass index, antibiotic use, PD-L1 expression, line of treatment and ECOG performance status), the TOPOSCORE had an independent relationship with overall survival in multivariate analyses (Table 16, HR=0.47 (0.33-0.67), p=0.001). Finally, the intraindividual dynamics of the TOPOSCORE in the same 96 NSCLC individuals sampled twice (baseline and 3 months) described above showed the relative stability of the SIG phenotype with 67% and 74% patients remaining in their SIG2+ and SIG1+ category respectively (FIG. 16C).

### Scoring Validation in Lung Cancer

We next applied the TOPOSCORE to a NSCLC validation cohort of 254 patients.

The proportions of patients falling into SIG1, Gray Akk^{high}, Akk⁰, Akk^{norm}, SIG2 were approximately similar to those described in the discovery cohort with 29%, 7%, 16%, 21%, 27% respectively (Table 15). Here, 44.2% and 22% of patients falling into S scoring≤0.5351 (SIG1) and S≥0.7911 (SIG2) presented an OS<12 months respectively (FIG. 16A, Table 15).

TABLE 15
Distribution of NSCLC and GU patients within the TOPOSCORE categorization.

| | | | | | Patients with | | | |
|---|---|---|---|---|---|---|---|---|
| | | | | Patients - | follow-up >12 | OS <12 - | OS >12- | p-value |
| Cohort | | Toposcore Category | | no (%) | months | no (%)* | no (%)* | (Chi-square) |
| NSCLC | Discovery | Toposcore | SIG 2 | 57 (23) | 52 (91.2) | 12 (23) | 40 (77) | <0.0001** |
| | (n = 245) | SIG2+ | Grey Akk^{L} | 48 (20) | 44 (91.6) | 17 (39) | 27 (61) | |
| | | Toposcore | SIG 1 | 74 (30) | 72 (97) | 50 (69) | 22 (31) | |
| | | SIG1+ | Grey Akk⁰ | 56 (23) | 52 (93) | 28 (54) | 24 (46) | |
| | | | Grey Akk^{H} | 10 (4) | 10 (100) | 5 (50) | 5 (50) | |
| | Validation | Toposcore | SIG 2 | 68 (27) | 52 (76) | 18 (35) | 34 (65) | 0.0186** |
| | (n = 254) | SIG2+ | Grey Akk^{L} | 54 (21) | 39 (72) | 13 (33) | 26 (67) | |
| | | Toposcore | SIG 1 | 73 (29) | 54 (74) | 28 (52) | 26 (48) | |
| | | SIG1+ | Grey Akk⁰ | 41 (16) | 28 (55) | 16 (57) | 12 (43) | |
| | | | Grey Akk^{H} | 18 (7) | 15 (83) | 6 (40) | 9 (60) | |
| GU | UC + RCC | Toposcore | SIG 2 | 59 (28) | 50 (84.7) | 18 (36) | 32 (64) | 0.0002** |
| | (n = 216) | SIG2+ | Grey Akk^{L} | 52 (24) | 36 (69.2) | 18 (50) | 18 (50) | |
| | | Toposcore | SIG 1 | 57 (27) | 49 (86) | 39 (80) | 10 (20) | |
| | | SIG1+ | Grey Akk⁰ | 45 (20) | 32 (71) | 17 (53) | 15 (47) | |
| | | | Grey Akk^{H} | 3 (1) | 3 (100) | 3 (100) | 0 (0) | |

*Percentage calculated in each category considering patients with follow-up >12 months
**Comparing SIG2 and Grey Akk^{L} vs SIG1 and Grey Akk⁰ and Akk^{H}

TABLE 16
Multivariate analyses of the TOPOSCORE in discovery NSCLC patients - Cox proportional-
hazards univariate and multivariate analyses for DISCOVERY cohort

| | Univariate analysis | | | | | Multivariate analysis¹ | | | | |
|---|---|---|---|---|---|---|---|---|---|---|
| | | | Hazard | Confidence | p- | | | Hazard | Confidence | p- |
| Variables | Groups | | ratio | interval 95% | value | Groups | | ratio | interval 95% | value |
| TOPOSCORE | SIG1+ | (n = 140) | Reference | | | SIG1+ | (n = 126) | Reference | | |
| | SIG2+ | (n = 105) | 0.471 | 0.340-0.652 | <0.001 | SIG2+ | (n = 88) | 0.476 | 0.334-0.677 | <0.001 |
| Age, per year | n = 245 | | 1 009 | 0.994-1.025 | 0.243 | n = 214 | | 1 014 | 0.997-1.031 | 0.102 |
| BMI | <18 | (n = 22) | Reference | | 0.003 | <18 | (n = 20) | Reference | | 0.002 |
| | [18-25) | n = 142) | 0.537 | 0.323-0.893 | 0.017 | 18-25 | (n = 124) | 0.545 | 0.324-0.915 | 0.022 |
| | ≥25 | (n = 81) | 0.395 | 0.230-0.680 | 0.001 | ≥25 | (n = 70) | 0.371 | 0.213-0.646 | <0.001 |
| Antibiotics | No | (n = 189) | Reference | | | No | (n = 166) | Reference | | |
| | Yes | (n = 56) | 1 813 | 1.279-2.579 | <0.001 | Yes | (n = 48) | 2 262 | 1.549-3.303 | <0.001 |
| ECOG-PS | 0-1 | (n = 174) | Reference | | | 0-1 | (n = 178) | Reference | | |
| | ≥2 | (n = 36) | 1 441 | 0.971-2.139 | 0.069 | ≥2 | (n = 36) | 1 286 | 0.857-1.928 | 0.224 |

| Treatment(s) | 1 | (n = 50) | Reference | | | Not included |
|---|---|---|---|---|---|---|
| line(s) | ≥2 | (n = 195) | 1 154 | 0.760-1.751 | 0.501 | |
| PD-L1 | 0-49% | (n = 60) | Reference | | | Not included |
| | ≥50% | (n = 66) | 1 128 | 0.705-1.803 | 0.616 | |
| Gender | Female | (n = 78) | Reference | | | Not included |
| | Male | (n = 159) | 1 101 | 0.789-1.537 | 0.572 | |

¹Total patients included = 214; missing data: 31; total events: 156

TABLE 17
Multivariate analyses of the TOPOSCORE in validation NSCLC patients - Cox proportional-
hazards univariate and multivariate analyses for VALIDATION cohort

| | Univariate analysis | | | | | Multivariate analysis¹ | | | | |
|---|---|---|---|---|---|---|---|---|---|---|
| | | | Hazard | Confidence | p- | | | Hazard | Confidence | p- |
| Variables | Groups | | ratio | interval 95% | value | Groups | | ratio | interval 95% | value |
| TOPOSCORE | SIG1+ | (n = 137) | Reference | | | SIG1+ | (n = 103) | Reference | | |
| | SIG2+ | (n = 117) | 0.557 | 0.374-0.830 | 0.004 | SIG2+ | (n = 90) | 0.595 | 0.362-0.978 | 0.041 |
| Age, per | n = 254 | | 1 314 | 0.894-1.93 | 0.165 | n = 193 | | 0.970 | 0.942-0.999 | 0.43 |
| year | | | | | | | | | | |
| BMI | <18 | (n = 17) | Reference | | 0.075 | <18 | (n = 11) | Reference | | 0.310 |
| | [18-25) | n = 132) | 2 326 | 0.223-1.744 | 0.102 | 18-25 | (n = 103) | 3 108 | 0.723-13.364 | 0.128 |
| | ≥25 | (n = 105) | 1 604 | 0.573-4.486 | 0.368 | ≥25 | (n = 79) | 2 864 | 0.651-12.605 | 0.164 |
| Antibiotics | No | (n = 217) | Reference | | | No | (n = 170) | Reference | | |
| | Yes | (n = 37) | 1 385 | 0.833-2.302 | 0.210 | Yes | (n = 23) | 0.961 | 0.462-2.001 | 0.916 |
| ECOG-PS | 0-1 | (n = 104) | Reference | | | 0-1 | (n = 170) | Reference | | |
| | ≥2 | (n = 147) | 1 631 | 1.085-2.452 | 0.019 | ≥2 | (n = 23) | 2 463 | 1.199-5.059 | 0.014 |
| Treatment(s) | 1 | (n = 98) | Reference | | | 1 | (n = 94) | Reference | | |
| line(s) | ≥2 | (n = 132) | 1 659 | 1.055-2.607 | 0.028 | ≥2 | (n = 99) | 1 275 | 0.723-2.250 | 0.401 |
| PD-L1 | 0-49% | (n = 73) | Reference | | | 0 | (n = 104) | Reference | | |
| | ≥50% | (n = 145) | 0.561 | 0.356-0.884 | 0.13 | ≥50% | (n = 89) | 0.632 | 0.350-1.141 | 0.128 |

| Gender | Female | (n = 98) | Reference | | | Not included |
|---|---|---|---|---|---|---|
| | Male | (n = 156) | 1 040 | 0.845-1.268 | 0.694 | |

¹Total patients included = 193; missing data: 61; total events: 72

As shown for the discovery cohort, the Cox regression analysis of the association of the TOPOSCORE with PFS and OS validated that the “SIG2+” (TOPOSCORE=1 or 2) category of patients exhibited a significantly prolonged clinical benefit to PD-1 blockade compared with the “SIG1+” (TOPOSCORE=3 to 5) subgroup (Table 15, FIG. 16B, p=0.058 for PFS, p=0.0034 for OS). Here again, the TOPOSCORE classifier represented an independent and more robust prognosis marker than PD-L1, age and antibiotics uptake in multivariate analyses (Table 17, HR=0.59 (0.36-0.97), p=0.041).

Importantly, pooling all NSCLC patients from the discovery and validation cohorts with an available PD-L1 immunohistochemical tumor labeling (n=344), we could demonstrate the added value of the TOPOSCORE not only in PD-L1 negative tumors but also in PD-L1 positive NSCLC patients (FIG. 16D, p=0.046 and p=0.032 respectively). The prognostic value of the TOPOSCORE was demonstrated not only in previously treated-patients but also in treatment-naïve individuals, being in anti-PD-1 Ab monotherapy in PD-L1>50% cases or in chemo-immunotherapy (FIG. 16E-F).

### Example 10: Prospective Validation of the TOPOSCORE in Other Cancer Cohorts Amenable to PD-1 Blockade

We next extended the use of the lung cancer-related TOPOSCORE to a new prospective cohort pooling 83 RCC (from ONCOBIOTICS) and 133 unorthelial cancer (UC) (from IOPREDI study) treated with anti-PD(L)1 antibodies in 2^{nd} L therapy, for which baseline samples and >6 months-clinical follow-up were available. The percentage of patients falling into SIG1+ for RCC and UC cohorts were 35% and 57%, respectively (FIG. 17A). Pooling all urinary tract malignancies, we found that the proportions of patients falling into SIG1, Gray Akk^{high}, Akk⁰, Akk^{norm}, SIG2 were approximately similar to those described in NSCLC with 26.4%, 1.4%, 21%, 24%, 27.3% respectively (Table 15). As found in NSCLC, 80% and 36% of RCC+UC patients within SIG1 and SIG2 had an OS<12 months, respectively (Table 15). The Cox regression analysis of the impact of the TOPOSCORE on PFS and OS confirmed that the “SIG1+” subset of patients harbors a reduced clinical benefit compared with the “SIG2+” subset (Table 15, FIG. 17B, left and right panels, p=0.0039 for PFS and p<0.0001 for OS).

Finally, we applied the TOPOSCORE to healthy individuals (HV) instead of cancer patients, computing the metagenomes of public databases (n=5345) and utilizing the MetaPhlAn 4.0 pipeline. To analyze the differences in the taxonomic stool composition between HV and the advanced NSCLC patients (segregated into OS> or <12 months) described above, we performed PCoA of Bray-Curtis distances on batch-corrected with MMUPHin (Ma et al. 2022) and normalized/standardized data that unveiled significant separation among HV and cancer groups. To determine the relative contribution of each MGS abundance at baseline to the observed three group separation, MGS were ordered according to their VIP score which relied on the supervised PLS-DA (FIG. 18A). Not surprisingly, 9 out of the 37 significant MGS were listed in SIG1 (such as Enterocloster clostridioformis, E. bolteae, Clostridium symbiosum . . . ) or SIG2 (Coprococcus comes, Dorea longicatena . . . ). In fact, the S score applied to HV highlighted that 68.5% were SIG2, 27.5% fell in the Gray zone and 4% were SIG1. Using the Akk trichotomic distribution, we could calculate that about 20% of HV are SIG1+ and could be considered a priori as inappropriate donors of fecal microbial transplantation (FIG. 17A, FIG. 18B).

Altogether, the TOPOSCORE allowed to conclude that 53%, 58%, 35% and 57% of 1^{st} L NSCLC, 2^{nd} L NSCLC, 1^{st}+2^{nd} L RCC, >2^{nd} L UC patients harbor a gut dysbiosis (defined by the percentage of SIG1+ individuals) in our cohorts (FIG. 17A) that was associated with immuno-resistance independently of other prognosis factors (Tables 16-17). In conclusion, the TOPOSCORE represents a robust biomarker predicting immune-sensitivity (and immune-resistance thereof) to ICI across lung and genitourinary tract cancer populations and a useful tool to follow the dynamics of gut dysbiosis on an individual basis.

### Example 11: Challenging the TOPOSCORE of Example 9 with Machine Learning Approaches

The Sensitivity, Specificity, Positive and Negative Predictive Values of the TOPOSCORE were calculated in the discovery cohort of NSCLC as 74.1%, 56.8%, 69.8% and 61.9%, respectively, with an AUC=0.66 [95% confidence interval 0.59-0.73]. This performance of the TOPOSCORE was compared with that of two machine-learning algorithms. First, Random Forest (RF) applied on relative abundances of all microbial species with SIAMCAT provided an AUC of 0.651±0.012 in the discovery cohort (FIG. 19A). Then, in an effort to evade the usual taxonomy-based RF modeling, we employed an innovative Metagenome Assembled Genomes (MAGs)-based RF model, which was built on relative abundances of 284 high-quality MAGs identified based on their capacity for keeping their interactions stable despite dramatic environmental perturbations. These 284 MAGs are organized as two competing guilds which support RF models for discriminating cases from controls across various disease states including insulin resistance and colon cancer (Wu et al. 2022). These 284 MAGs were used to construct a RF model for predicting personalized immunoresistance with an AUC of 0.69 (95% Cl 0.62-0.76) in this discovery cohort (FIG. 19B).

Thus, the referenced machine-learning algorithms gave similar results in terms of individual prediction in the discovery cohort compared to the TOPOSCORE.

### Example 12: Functional Pathways Associated with SIG1 and SIG2 MGS

To explore putative microbial functions underlying SIG1 and SIG2 compositions, we employed an analysis of MG pathways by means of HUMAnN 3.0 pipeline. This pipeline first annotates microbial-specific gene hits according to the Kyoto Encyclopedia of Genes and Genomes Orthology, then reconstructs microbial metabolic pathways using the MetaCyc hierarchy. We thus retrieved 664 pathways (unclassified excluded, and 441 at 20% prevalence cutoff) in the whole cohort of NSCLC 499 patients, with 11 and 57 pathways exclusively present in SIG1 and SIG2 microbial communities, respectively, and 76 shared pathways (for a total of 144 pathways) (FIG. 19C, Table 18). PLS-DA ordination plot showed significant compositional differences in the functional pathways across sample types among SIG1+ and SIG2+ patients, while the VIP plot showed discriminant and significant pathways for each cohort (FIG. 19D-E). SIG2 metabolic functions encompass sulfur oxidation, tRNA charging and processing, stachyose and mannan degradation, L-glutamate and L glutamine as well as L-Arginine and L-ornithine biosynthesis, L-tryptophane and dTDP-L-rhamnose_biosynthesis, as well as the pentose phosphate pathways. In contrast, SIG1 metabolic functions gather fatty acid betaoxidation, 5′deoxyadenosine and L phenylalanine degradation, purine and L-histidine degradation and guanosine and L-lysine biosynthesis. A hierarchical clustering based on the overall 144 pathway abundances related to SIG1 and SIG2 MGS, was applied to the 499 NSCLC patients, showing a clear separation of patients into two distinct groups, cluster 1 harboring 73% of SIG1+ individuals enriched in SIG1 functional pathways and cluster 2 harboring 70% of SIG2+ patients enriched in SIG2 functional pathways (X² statistic with Yates correction=88.305, p<0.00001). Thus, even if 76 pathways are in common among SIG1 and SIG2 groups of microbial species, the overall 144 pathway distribution mirrored the seesaw balance between these interactive groups, dividing them into two distinct functional patterns related to SIG1 and SIG2 MGS functions (Fisher exact test<0.00001).

TABLE 18
List of pathways distinctive for SIG1 and SIG2

[SIG1] functional pathways

| | |
|---|---|
| γ-glutamyl cycle | pyrimidine deoxyribonucleotide phosphorylation |
| L-histidine degradation I | sucrose degradation IV (sucrose phosphorylase) |
| L-lysine biosynthesis II | superpathway of guanosine nucleotides de novo |
| | biosynthesis I |
| O-antigen building blocks biosynthesis (E. coli) | superpathway of guanosine nucleotides de novo |
| | biosynthesis II |
| inosine-5′-phosphate biosynthesis III | superpathway of purine deoxyribonucleosides |
| | degradation |
| purine nucleobases degradation I (anaerobic) | |

[SIG2] functional pathways

| | |
|---|---|
| D-fructuronate degradation | methanogenesis from acetate |
| D-galactose degradation I (Leloir pathway) | methylerythritol phosphate pathway I |
| D-galacturonate degradation I | molybdopterin biosynthesis |
| L-homoserine and L-methionine biosynthesis | myo-, chiro- and scillo-inositol degradation |
| L-isoleucine biosynthesis IV | pantothenate and coenzyme A biosynthesis I |
| L-methionine biosynthesis I | pentose phosphate pathway (non-oxidative branch) I |
| L-ornithine biosynthesis I | pentose phosphate pathway (non-oxidative branch) II |
| NAD biosynthesis I (from aspartate) | phosphatidylglycerol biosynthesis I (plastidic) |
| S-adenosyl-L-methionine salvage I | phosphatidylglycerol biosynthesis II (non-plastidic) |
| UDP-N-acetylmuramoyl-pentapeptide biosynthesis | phosphopantothenate biosynthesis I |
| III (meso-diaminopimelate containing) | |
| UMP biosynthesis I | purine nucleotides degradation II (aerobic) |
| UMP biosynthesis II | putrescine biosynthesis IV |
| UMP biosynthesis III | queuosine biosynthesis I (de novo) |
| acetyl-CoA fermentation to butanoate II | sucrose biosynthesis II |
| adenosine nucleotides degradation II | superpathway of β-D-glucuronide and D- |
| | glucuronate degradation |
| adenosylcobalamin salvage from cobinamide I | superpathway of L-alanine biosynthesis |
| coenzyme A biosynthesis I (prokaryotic) | superpathway of L-aspartate and L-asparagine |
| | biosynthesis |
| coenzyme A biosynthesis II (eukaryotic) | superpathway of N-acetylglucosamine, N- |
| | acetylmannosamine and N-acetylneuraminate |
| | degradation |
| dTDP-β-L-rhamnose biosynthesis | superpathway of S-adenosyl-L-methionine |
| | biosynthesis |
| dTDP-N-acetylthomosamine biosynthesis | superpathway of adenosylcobalamin salvage from |
| | cobinamide I |
| fatty acid biosynthesis initiation (mitochondria) | superpathway of branched chain amino acid |
| | biosynthesis |
| flavin biosynthesis III (fungi) | superpathway of coenzyme A biosynthesis III |
| | (mammals) |
| formaldehyde assimilation II (RuMP Cycle) | superpathway of glycerol degradation to 1,3- |
| | propanediol |
| glutaryl-CoA degradation | superpathway of sulfur oxidation (Acidianus |
| | ambivalens) |
| glycogen degradation II | superpathway of thiamin diphosphate biosynthesis |
| | III (eukaryotes) |
| glycolysis IV | thiamin salvage II |
| guanosine nucleotides degradation II | thiamine phosphate formation from pyrithiamine |
| | and oxythiamine (yeast) |
| hexitol fermentation to lactate, formate, ethanol | thiazole biosynthesis I (E. coli) |
| and acetate | |
| inosine 5′-phosphate degradation | |

### Example 13: Development of a New User-Friendly gPCR-Based TOPOSCORE Assay

Finally, to transform the TOPOSCORE into a clinically actionable diagnosis tool, we contemplated to circumvent the costly, laborious, and time-consuming method of shotgun metagenomics by means of a qPCR-based assay that can be performed within 48 hours for determining bacteria prevalence. Based on the most prevalent MGS species found for the gut oncomicrobial signatures across various cohorts (Park et al. 2022; Thomas et al. 2023) and the feasibility of designing bacteria-specific and reliable probe sets (FIG. 20), we focused our qPCR-based assay on 15 SIG2- and 5 SIG1-associated bacteria in addition to Akkermansia spp. (FIG. 21). First, we attempted to re-run the TOPOSCORE calculation for the whole NSCLC cohort based on the MGS-based relative abundances of these 21 microbial species (instead of the full set of 83 bacteria).

In FIG. 17C left panel, we show the survival curve of the 393 NSCLC patients confirming that an 83 MGS-based TOPOSCORE can discriminate between favorable versus dismal prognosis. A comparable performance was obtained using the 21 MGS-based TOPOSCORE determined by shotgun MG analyses (FIG. 17C, right panel). Importantly, Spearman correlation indices between MGS relative abundance (and therefore prevalence) and PCR quantification of each of the 21 bacterium-specific DNA validated the strategy of switching from shotgun MG to qPCR (two examples are shown for two bacteria (FIG. 17D), refer to FIG. 21 for the other 19 bacteria). Lastly, when utilizing the 21 bacteria-specific probe sets on the remaining fecal DNA (originally extracted for shotgun MG) from the whole NSCLC cohort (n=313), we confirmed that OS was superior in those patients harboring a qPCR-based TOPOSCORE falling within the SIG2+ category (FIG. 17E, p=0.0015). We validated the robustness of the qPCR-based TOPOSCORE to predict OS in a prospective cohort of 96 NSCLC patients (FIG. 17F).

In conclusion, we demonstrated that a quick test, easily translatable into clinical routine, is feasible and reliable to predict survival during immunotherapy of lung cancer.

### DISCUSSION about Examples 9-13

Despite the use of microbiome-trained machine learning across different geographical cohorts, consistent prediction of PD-(L)1 therapy outcomes remains elusive. Notably, there has not been a reproducible microbiome signature to reliably predict individual clinical outcomes. This observation is consistent with three meta-analyses spanning various cancer types and therapies that failed to resolve discrepancies in existing cohorts (Gharaibeh and Jobin 2019; Limeta et al. 2020; Shaikh et al. 2021). Recently, two meta-analyses focused on melanoma utilized shotgun MG databases and machine learning methodology to offer partial clarity on the “microbiotypes” associated with responses or resistance to immunotherapy (Lee et al. 2022; McCulloch et al. 2022).

In the present study, we pivot to an ecosystem-based strategy. Hence, our work suggests gut residence of cooperative ecosystems yielding consistent co-abundance patterns harboring opposite clinical relevance (sensitivity versus resistance to ICI) in a seesaw manner. Computing the TOPOSCORE on 715 advanced cancer patients, we found that around 50% of individuals could be classified within SIG1+ among whom about 63% had an OS<12 months.

The prevalence of each SIG1 member is lower than that of each SIG2 member. Around 50% of SIG1 MGS have a prevalence<15% while about 55% of SIG2 MGS have a prevalence >50% in HV and cancer patients (FIG. 20). SIG1 contained 37 species belonging to the Enterocloster genus, Streptococcaceae, Veillonellaceae and Lactobacillaceae families, already identified in immune resistant patients (Gilbert et al. 2018; L. Derosa et al. 2018; Mager et al. 2020; Overacre-Delgoffe et al. 2021). In fact, SIG1 comprises many microbial components of the oral cavity, that can transit from the supraglottic compartment down to the bronchoalveolar space or the small intestine, as a result of pH fluctuations and/or co-medications (proton pump inhibitors) or dysphagia (Lee et al. 2022; Tsay et al. 2020; Cortellini et al. 2020; Imhann et al. 2016; Jackson et al. 2018). Oralization of the intestinal microbiota has been linked to failure of immunotherapy and immune-related adverse events (McCulloch et al. 2022), (Cosseau et al. 2008; Bernardo et al. 2012; Santos Rocha et al. 2012). Veillonella spp. have the capacity to expand TH17 pro-angiogenic and -oncogenic lymphocytes that contribute to dismal prognosis and resistance to cytotoxicants in NSCLC (Tsay et al. 2020). The Enterocloster genus (E. aldensis, E. asparagiformis, E. bolteae) represents a vancomycin-sensitive clade of immunosuppressive bacteria, dominant in the intestinal microbiota of people and patients suffering from aging and chronic inflammatory disorders including cancer (Limeta et al. 2020; Ghosh et al. 2020). By causing a beta-adrenergic receptor-dependent stress ileopathy and an Enterocloster genus-dominated dysbiosis, some malignancies (and other pathological disorders such as stroke) may increase gut permeability, favoring translocation of inflammatory mediators and bacteremia with immunosuppressive potential (Yonekura et al. 2021; Stanley et al. 2016). Interestingly, the Gut Microbiome Health Index (GMHI), an index proposed to predict the disease likelihood of an individual based on its fecal microbial composition (Gupta et al. 2020) found species in the non-healthy group similar to the ones retrieved here in SIG1, but none of the species falling within the healthy group were retrieved in our SIG2, mainly because NSCLC patients are already diseased. Instead, SIG2 was composed of 45 species gathering Lachnospiraceae and Oscillospiraceae family members largely reported for their strong association with healthy status and favorable clinical responses (Yonekura et al. 2021; Gopalakrishnan et al. 2018; Spencer et al. 2021; McCulloch et al. 2022; Frankel et al. 2017; Chaput et al. 2017). Indeed, SIG2 functional fingerprints (stachyose degradation (Spencer et al. 2021), biosynthesis of L-ornithine and L-Arginine of polyamine pathway (Danlos et al. 2021; Peyraud et al. 2022; Geiger et al. 2016; Canale et al. 2021), purine ribonucleoside degradation (Teng et al. 2023) plead in favor of metabolic patterns that could keep in check tumor cell cycle and reactivate immunosurveillance.

Sensitivity, specificity, positive and negative predictive values of the TOPOSCORE were 74.1%, 56.8%, 69.8% and 61.9%, respectively, with an AUC=0.66 [95% confidence interval 0.59-0.73]. Of note, the alternative state-of-the-art machine learning algorithms (including SIRUS, SIAMCAT and MAGs-based RF) performed equally well (FIG. 19). The robustness of the TOPOSCORE was not only illustrated in NSCLC but also in stage IV urinary tract bearing cancer patients amenable to ICI (including 83 (1L+2L) RCC, and 133 (>2L) UC. Interestingly, the fraction of SIG1+ patients was lower in RCC (34%) than in UC (57%), suggesting that platinum salt-based chemotherapy might be less efficient than tyrosine kinase inhibitors to prevent gut dysbiosis.

Indeed, the longitudinal scoring of patients will be instrumental to understand the impact of each therapy on the gut homeostasis. Likewise, the TOPOSCORE can also represent a valuable tool to select donors of fecal microbial transplantation (FMT). It is interesting to outline that about 21% of HV fell into the SIG1+ category, suggesting that the TOPOSCORE could help dismiss donor candidates of FMT in favor of the 26% fraction (1399/5345) that resides in the top 10% of the TOPOSCORE (0.90). In contrast, only 6% cancer patients (43/715) scored 0.90 and could theoretically be preferentially selected for FMT. The TOPOSCORE also covers the unmet medical need of patient stratification based on “gut dysbiosis” in order to ascribe resistance to ICI to an objective deviation from the “healthy” taxonomic composition (rather than to a cell-intrinsic molecular cue), and to guide the outcome of microbiota-centered interventions. Hence, the TOPOSCORE represents an actionable diagnosis tool for the pharmacodynamics of live biotherapeutics, FMT and prebiotics. More specifically, the TOPOSCORE offers a friendly user process to quickly assess gut dysbiosis in a given individual at any time of the disease. Indeed, we showed that it was possible to obtain comparable results using a 21 bacteria-probe set-based qPCR rather than a 83 MGS-based shotgun MG TOPOSCORE, leveraging this diagnosis test within the routine tool-box. Admittedly, incrementation of additional MGS into the qPCR-based TOPOSCORE may improve its performance. The TOPOSCORE may fluctuate with patient accrual, disease selection and geography. Of note, the TOPOSCORE was computed based on 12 months-overall survival, suggesting that it may not be helpful to predict response rates at the first CT scan.

Despite these limitations, our work offers a new method of dimension reduction of clinical relevance to assess gut dysbiosis in cancer patients amenable to immunotherapy.

### Example 14: Use of the TOPOSCORE Measured as Described in Example 13 in Colorectal Cancer

The TOPOSCORE of patients enrolled in ATEZOTRIBE clinical trial was assessed using the 21 MGS-based PCR asay of Example 13.

ATEZOTRIBE is a randomised clinical trial with 150 colorectal cancers in two arms, with or without anti-PDL-1 Ab (atezolizumab).

As shown in FIG. 22 (only MSS patients shown) the TOPOSCORE predicts overall survival only in the immunotherapy (anti-PDL-1 Ab) arm (not the chemotherapy arm).

### Example 15: Further Examples of gPCR-Based TOPOSCORE Assays

Ten combinations of 50 bacterial species, each differring by the one species, were used to calculate the TOPOSCORE of NSCLC patients (N=20).

The 49 bacterial species common to all of these combinations are Ruminococcus bicirculans, Faecalibacterium prausnitzii, Blautia wexlerae, Roseburia intestinalis, Gemmiger formicilis, Anaerostipes hadrus, Streptococcus parasanguinis, Clostridiales bacterium KLE1615, Agathobaculum butyriciproducens, Dorea longicatena, Clostridium symbiosum, Blautia massiliensis, Eubacterium rectale, Faecalibacterium SGB15346, Clostridium sp AF34 10BH, Lachnospira eligens, Lachnospiraceae bacterium WCA3 601 WT 6H, Streptococcus salivarius, Clostridium fessum, Anaerobutyricum hallii, Hungatella hathewayi, Candidatus Cibiobacter qucibialis, Anaerotignum faecicola, Clostridium scindens, Clostridium innocuum, Clostridiaceae unclassified SGB4769, Roseburia hominis, Clostridiaceae bacterium, Oscillibacter sp ER4, Clostridiaceae bacterium OM08 6BH, Roseburia inulinivorans, Phocaeicola massiliensis, Enterocloster aldensis, Veillonella parvula, Lacrimispora amygdalina, Firmicutes bacterium AF16 15, Coprococcus eutactus, Eubacterium ventriosum, Enterocloster bolteae, Clostridiales unclassified SGB15145, Faecalibacillus intestinalis, Coprococcus comes, Roseburia sp AF02 12, Erysipelatoclostridium ramosum, Clostridium sp AM49 4BH, Mediterraneibacter butyricigenes, Dorea formicigenerans, Coprobacter fastidiosus ad Enterocloster clostridioformis.

In each assay, one additional bacterial species was used in addition to the above 49 species. These variable species were:
J1: Ruminococcus lactaris,
J2: Bifidobacterium dentium,
J3: Lachnospira sp NSJ 43,
J4: Clostridium sp AM22 11AC,
J5: Lachnospira pectinoschiza,
J6: Lachnospiraceae bacterium OM04 12BH,
J7: Clostridium sp AM33 3,
J8: Veillonella dispar,
J9: Eubacterium ramulus and
J10: Actinomyces graevenitzii.

Akkermansia was also used to refine the TOPOSCORE in the grey zone.

The AUC obtained with these combinations of bacterial species are shown in FIG. 23.

### REFERENCES

Bénard, Clément, Gérard Biau, Sébastien Da Veiga, and Erwan Scornet. 2021.«SIRUS: Stable and Interpretable RUle Set for classification». Electronic Journal of Statistics 15 (1): 427-505.
Bernardo, David, Borja Sanchez, Hafid O. AI-Hassi, Elizabeth R. Mann, Maria C. Urdaci, Stella C. Knight, and Abelardo Margolles. 2012.«Microbiota/Host Crosstalk Biomarkers: Regulatory Response of Human Intestinal Dendritic Cells Exposed to Lactobacillus Extracellular Encrypted Peptide». PLOS ONE 7 (5): e36262.
Carbonero, Franck, Ann Benefiel, Amir Alizadeh-Ghamsari, and H. Rex Gaskins. 2012.«Microbial pathways in colonic sulfur metabolism and links with health and disease». Frontiers in Physiology 3.
Chang, Chih-Chung, and Chih-Jen Lin. 2011.«LIBSVM: A library for support vector machines». ACM Transactions on Intelligent Systems and Technology 2 (3), 27, 1-27.
Chaput, N., P. Lepage, C. Coutzac, E. Soularue, K. Le Roux, C. Monot, L. Boselli, et al. 2017. «Baseline Gut Microbiota Predicts Clinical Response and Colitis in Metastatic Melanoma Patients Treated with Ipilimumab». Annals of Oncology: Official Journal of the European Society for Medical Oncology 28 (6): 1368-79.
Cho, Ilseung, and Martin J. Blaser. 2012.«The Human Microbiome: At the Interface of Health and Disease». Nature Reviews. Genetics 13 (4): 260-70.
Clark, Ryan L., Bryce M. Connors, David M. Stevenson, Susan E. Hromada, Joshua J. Hamilton, Daniel Amador-Noguez, and Ophelia S. Venturelli. 2021.«Design of Synthetic Human Gut Microbiome Assembly and Butyrate Production». Nature Communications 12 (1): 3254.
Cortellini, Alessio, Marco Tucci, Vincenzo Adamo, Luigia Stefania Stucci, Alessandro Russo, Enrica Teresa Tanda, Francesco Spagnolo, et al. 2020.«Integrated Analysis of Concomitant Medications and Oncological Outcomes from PD-1/PD-L1 Checkpoint Inhibitors in Clinical Practice». Journal for Immunotherapy of Cancer 8 (2): e001361.
Cosseau, Celine, Deirdre A. Devine, Edie Dullaghan, Jennifer L. Gardy, Avinash Chikatamarla, Shaan Gellatly, Lorraine L. Yu, et al. 2008.«The Commensal Streptococcus Salivarius K12 Downregulates the Innate Immune Responses of Human Epithelial Cells and Promotes Host-Microbe Homeostasis». Infection and Immunity 76 (9): 4163-75.
Davar, Diwakar, Amiran K. Dzutsev, John A. McCulloch, Richard R. Rodrigues, Joe-Marc Chauvin, Robert M. Morrison, Richelle N. Deblasio, et al. 2021.«Fecal Microbiota Transplant Overcomes Resistance to Anti-PD-1 Therapy in Melanoma Patients». Science 371 (6529): 595-602.
Derosa, L., M. D. Hellmann, M. Spaziano, D. Halpenny, M. Fidelle, H. Rizvi, N. Long, et al. 2018.«Negative Association of Antibiotics on Clinical Activity of Immune Checkpoint Inhibitors in Patients with Advanced Renal Cell and Non-Small-Cell Lung Cancer». Annals of Oncology: Official Journal of the European Society for Medical Oncology 29 (6): 1437-44.
Derosa, Lisa, Bertrand Routy, Marine Fidelle, Valerio lebba, Laurie Alla, Edoardo Pasolli, Nicola Segata, et al. 2020.«Gut Bacteria Composition Drives Primary Resistance to Cancer Immunotherapy in Renal Cell Carcinoma Patients». European Urology 78 (2): 195-206.
Derosa, Lisa, Bertrand Routy, Guido Kroemer, and Laurence Zitvogel. 2018.«The Intestinal Microbiota Determines the Clinical Efficacy of Immune Checkpoint Blockers Targeting PD-1/PD-L1». Oncoimmunology 7 (6): e1434468.
Derosa, Lisa, Bertrand Routy, Laurence Zitvogel, Andrew M. Thomas, Gerard Zalcman, Sylvie Friard, Julien Mazieres, et al. 2021.«Intestinal Akkermansia muciniphila predicts overall survival in advanced non-small cell lung cancer patients treated with anti-PD-1 antibodies: Results a phase II study». Journal of Clinical Oncology, Volume 39, Issue 15 suppl, 9019-9019.
Derosa, Lisa, Bertrand Routy, Andrew Maltez Thomas, Valerio lebba, Gerard Zalcman, Sylvie Friard, Julien Mazieres, et al. 2022a. “Intestinal Akkermansia Muciniphila Predicts Clinical Response to PD-1 Blockade in Patients with Advanced Non-Small-Cell Lung Cancer.” Nature Medicine 28 (2): 315-24.
Derosa, Lisa et al. 2022b. “Intestinal Akkermansia Muciniphila Predicts Clinical Response to PD-1 Blockade in Patients with Advanced Non-Small-Cell Lung Cancer.” Nature Medicine, February.
Dizman, Nazli, Luis Meza, Paulo Bergerot, Marice Alcantara, Tanya Dorff, Yung Lyou, Paul Frankel, et al. 2022.«Nivolumab plus Ipilimumab with or without Live Bacterial Supplementation in Metastatic Renal Cell Carcinoma: A Randomized Phase 1 Trial». Nature Medicine 28 (4): 704-12.
Dordević, Dani, Simona Jancikova, Monika Vitézové, and Ivan Kushkevych. 2021. «Hydrogen Sulfide Toxicity in the Gut Environment: Meta-Analysis of Sulfate-Reducing and Lactic Acid Bacteria in Inflammatory Processes». Journal of Advanced Research, Chemistry, Biology and Clinical Applications of the Third Gasotransmitter, Hydrogen Sulfide (H2S), 55-69.
Frankel, Arthur E., Laura A. Coughlin, Jiwoong Kim, Thomas W. Froehlich, Yang Xie, Eugene P. Frenkel, and Andrew Y. Koh. 2017.«Metagenomic Shotgun Sequencing and Unbiased Metabolomic Profiling Identify Specific Human Gut Microbiota and Metabolites Associated with Immune Checkpoint Therapy Efficacy in Melanoma Patients». Neoplasia (New York, N. Y) 19 (10): 848-55.
Friedman, Jonathan, Logan M. Higgins, and Jeff Gore. 2017.«Community Structure Follows Simple Assembly Rules in Microbial Microcosms». Nature Ecology & Evolution 1 (5): 1-7.
Gacesa, R., A. Kurilshikov, A. Vich Vila, T. Sinha, M. a. Y. Klaassen, L. A. Bolte, S. Andreu-Sánchez, et al. 2022.«Environmental Factors Shaping the Gut Microbiome in a Dutch Population». Nature 604 (7907): 732-39.
Gharaibeh, Raad Z., and Christian Jobin. 2019.«Microbiota and Cancer Immunotherapy: In Search of Microbial Signals». Gut 68 (3): 385-88.
Ghosh, Tarini S., Mrinmoy Das, Ian B. Jeffery, and Paul W. O'Toole. 2020.«Adjusting for Age Improves Identification of Gut Microbiome Alterations in Multiple Diseases». ELife 9, e50240.
Gilbert, Jack A., Martin J. Blaser, J. Gregory Caporaso, Janet K. Jansson, Susan V. Lynch, and Rob Knight. 2018.«Current Understanding of the Human Microbiome». Nature Medicine 24 (4): 392-400.
Gopalakrishnan, V., C. N. Spencer, L. Nezi, A. Reuben, M. C. Andrews, T. V. Karpinets, P. A. Prieto, et al. 2018.«Gut Microbiome Modulates Response to Anti-PD-1 Immunotherapy in Melanoma Patients». Science (New York, N.Y.) 359 (6371): 97-103.
Gupta, Vinod K., Minsuk Kim, Utpal Bakshi, Kevin Y. Cunningham, John M. Davis, Konstantinos N. Lazaridis, Heidi Nelson, Nicholas Chia, and Jaeyun Sung. 2020.«A Predictive Index for Health Status Using Species-Level Gut Microbiome Profiling». Nature Communications 11 (1): 4635.
Heng, Daniel Y. C., Wanling Xie, Meredith M. Regan, Mark A. Warren, Ali Reza Golshayan, Chakshu Sahi, Bernhard J. Eigl, et al. 2009.«Prognostic Factors for Overall Survival in Patients with Metastatic Renal Cell Carcinoma Treated with Vascular Endothelial Growth Factor-Targeted Agents: Results from a Large, Multicenter Study». Journal of Clinical Oncology: Official Journal of the American Society of Clinical Oncology, 27 (34): 5794-99.
Imhann, Floris, Marc Jan Bonder, Arnau Vich Vila, Jingyuan Fu, Zlatan Mujagic, Lisa Vork, Ettje F. Tigchelaar, et al. 2016.«Proton Pump Inhibitors Affect the Gut Microbiome». Gut 65 (5): 740-48.
Jackson, Matthew A., Serena Verdi, Maria-Emanuela Maxan, Cheol Min Shin, Jonas Zierer, Ruth C. E. Bowyer, Tiphaine Martin, et al. 2018.«Gut Microbiota Associations with Common Diseases and Prescription Medications in a Population-Based Cohort». Nature Communications 9 (1): 2655.
Kawashima, Tadaomi, Akemi Kosaka, Huimin Yan, Zijin Guo, Ryosuke Uchiyama, Ryutaro Fukui, Daisuke Kaneko, et al. 2013.«Double-Stranded RNA of Intestinal Commensal but Not Pathogenic Bacteria Triggers Production of Protective Interferon-β». Immunity 38 (6): 1187-97.
Lee, Karla A., Andrew Maltez Thomas, Laura A. Bolte, Johannes R. Björk, Laura Kist de Ruijter, Federica Armanini, Francesco Asnicar, et al. 2022.«Cross-Cohort Gut Microbiome Associations with Immune Checkpoint Inhibitor Response in Advanced Melanoma». Nature Medicine 28 (3): 535-44.
Li, Min, Baohong Wang, Menghui Zhang, Mattias Rantalainen, Shengyue Wang, Haokui Zhou, Yan Zhang, et al. 2008.«Symbiotic Gut Microbes Modulate Human Metabolic Phenotypes». Proceedings of the National Academy of Sciences of the United States of America 105 (6): 2117-22.
Limeta, Angelo, Boyang Ji, Max Levin, Francesco Gatto, and Jens Nielsen. 2020.«Meta-Analysis of the Gut Microbiota in Predicting Response to Cancer Immunotherapy in Metastatic Melanoma». JCI Insight, 5 (23).
Mager, Lukas F., Regula Burkhard, Nicola Pett, Noah C. A. Cooke, Kirsty Brown, Hena Ramay, Seungil Paik, et al. 2020.«Microbiome-Derived Inosine Modulates Response to Checkpoint Inhibitor Immunotherapy». Science (New York, N. Y) 369 (6510): 1481-89.
McCulloch, John A., Diwakar Davar, Richard R. Rodrigues, Jonathan H. Badger, Jennifer R. Fang, Alicia M. Cole, Ascharya K. Balaji, et al. 2022.«Intestinal Microbiota Signatures of Clinical Response and Immune-Related Adverse Events in Melanoma Patients Treated with Anti-PD-1». Nature Medicine 28 (3): 545-56.
Messaoudene, Meriem, Reilly Pidgeon, Corentin Richard, Mayra Ponce, Khoudia Diop, Myriam Benlaifaoui, Alexis Nolin-Lapalme, et al. 2022.«A Natural Polyphenol Exerts Antitumor Activity and Circumvents Anti-PD-1 Resistance through Effects on the Gut Microbiota». Cancer Discovery 12 (4): 1070-87.
Newsome, Rachel C., Raad Z. Gharaibeh, Christine M. Pierce, Wildson Vieira da Silva, Shirlene Paul, Stephanie R. Hogue, Qin Yu, et al. 2022.«Interaction of Bacterial Genera Associated with Therapeutic Response to Immune Checkpoint PD-1 Blockade in a United States Cohort». Genome Medicine 14 (1): 35.
Overacre-Delgoffe, Abigail E., Hannah J. Bumgarner, Anthony R. Cillo, Ansen H. P. Burr, Justin T. Tometich, Amrita Bhattacharjee, Tullia C. Bruno, Dario A. A. Vignali, and Timothy W. Hand. 2021.«Microbiota-Specific T Follicular Helper Cells Drive Tertiary Lymphoid Structures and Anti-Tumor Immunity against Colorectal Cancer». Immunity 54 (12): 2812-2824.e4.
Park, Elizabeth M., Manoj Chelvanambi, Neal Bhutiani, Guido Kroemer, Laurence Zitvogel, and Jennifer A. Wargo. 2022.«Targeting the Gut and Tumor Microbiota in Cancer». Nature Medicine 28 (4): 690-703.
Peschel, Stefanie, Christian L Müller, Erika von Mutius, Anne-Laure Boulesteix, and Martin Depner. 2021.«NetCoMi: network construction and comparison for microbiome data in R». Briefings in Bioinformatics 22 (4): bbaa290.
Roberti, Maria Paula, Satoru Yonekura, Connie P. M. Duong, Marion Picard, Gladys Ferrere, Maryam Tidjani Alou, Conrad Rauber, et al. 2020.«Chemotherapy-Induced Ileal Crypt Apoptosis and the Ileal Microbiome Shape Immunosurveillance and Prognosis of Proximal Colon Cancer». Nature Medicine 26 (6): 919-31.
Routy, Bertrand, Emmanuelle Le Chatelier, Lisa Derosa, Connie P. M. Duong, Maryam Tidjani Alou, Romain Daillere, Aurélie Fluckiger, et al. 2018.«Gut Microbiome Influences Efficacy of PD-1-Based Immunotherapy against Epithelial Tumors». Science (New York, N. Y.) 359 (6371): 91-97.
Sanchez-Gorostiaga, Alicia, Djordje Bajid, Melisa L. Osborne, Juan F. Poyatos, and Alvaro Sanchez. 2019.«High-Order Interactions Distort the Functional Landscape of Microbial Consortia». PLoS Biology 17 (12): e3000550.
Santos Rocha, Clarissa, Omar Lakhdari, Hervé M. Blottiere, Sébastien Blugeon, Harry Sokol, Luis G. Bermudez-Humaran, Vasco Azevedo, et al. 2012.«Anti-Inflammatory Properties of Dairy Lactobacilli». Inflammatory Bowel Diseases 18 (4): 657-66.
Shaikh, Fyza Y., James R. White, Joell J. Gills, Taiki Hakozaki, Corentin Richard, Bertrand Routy, Yusuke Okuma, et al. 2021.«A Uniform Computational Approach Improved on Existing Pipelines to Reveal Microbiome Biomarkers of Nonresponse to Immune Checkpoint Inhibitors». Clinical Cancer Research 27 (9): 2571-83.
Smith, Christof C., Kathryn E. Beckermann, Dante S. Bortone, Aguirre A. De Cubas, Lisa M. Bixby, Samuel J. Lee, Anshuman Panda, et al. 2018.«Endogenous Retroviral Signatures Predict Immunotherapy Response in Clear Cell Renal Cell Carcinoma». The Journal of Clinical Investigation 128 (11): 4804-20.
Smith, Melody, Anqi Dai, Guido Ghilardi, Kimberly V. Amelsberg, Sean M. Devlin, Raymone Pajarillo, John B. Slingerland, et al. 2022.«Gut Microbiome Correlates of Response and Toxicity Following Anti-CD19 CAR T Cell Therapy». Nature Medicine 28 (4): 713-23.
Sonpavde, Guru P., Cora N. Sternberg, Yohann Loriot, Aurelien Marabelle, Jae Lyun Lee, Aude Fléchon, Guilhem Roubaud, et al. 2022. “Primary Results of STRONG: An Open-Label, Multicenter, Phase 3b Study of Fixed-Dose Durvalumab Monotherapy in Previously Treated Patients with Urinary Tract Carcinoma.” European Journal of Cancer 163
Spencer, Christine N., Jennifer L. McQuade, Vancheswaran Gopalakrishnan, John A. McCulloch, Marie Vetizou, Alexandria P. Cogdill, Md A. Wadud Khan, et al. 2021. ((Dietary Fiber and Probiotics Influence the Gut Microbiome and Melanoma Immunotherapy Response». Science (New York, N. Y) 374 (6575): 1632-40.
Stanley, Dragana, Linda J. Mason, Kate E. Mackin, Yogitha N. Srikhanta, Dena Lyras, Monica D. Prakash, Kulmira Nurgali, et al. 2016.«Translocation and Dissemination of Commensal Bacteria in Post-Stroke Infection». Nature Medicine 22 (11): 1277-84.
Teng, Huajing, Yan Wang, Xin Sui, Jiawen Fan, Shuai Li, Xiao Lei, Chen Shi, et al. 2023. “Gut Microbiota-Mediated Nucleotide Synthesis Attenuates the Response to Neoadjuvant Chemoradiotherapy in Rectal Cancer.” Cancer Cell 41 (1).
Terrisse, Safae, Lisa Derosa, Valerio lebba, Frangois Ghiringhelli, Ines Vaz-Luis, Guido Kroemer, Marine Fidelle, et al. 2021.«Intestinal Microbiota Influences Clinical Outcome and Side Effects of Early Breast Cancer Treatment». Cell Death & Differentiation, 1-19.
Thomas, Andrew Maltez, Marine Fidelle, Bertrand Routy, Guido Kroemer, Jennifer A. Wargo, Nicola Segata, and Laurence Zitvogel. 2023. “Gut OncoMicrobiome Signatures (GOMS) as next-Generation Biomarkers for Cancer Immunotherapy.” Nature Reviews. Clinical Oncology, June.
Tsay, Jun-Chieh J., Benjamin G. Wu, Imran Sulaiman, Katherine Gershner, Rosemary Schluger, Yonghua Li, Ting-An Yie, et al. 2021.«Lower Airway Dysbiosis Affects Lung Cancer Progression». Cancer Discovery, 11(2): 293-307.
Vétizou, Marie, Jonathan M. Pitt, Romain Daillere, Patricia Lepage, Nadine Waldschmitt, Caroline Flament, Sylvie Rusakiewicz, et al. 2015.«Anticancer Immunotherapy by CTLA-4 Blockade Relies on the Gut Microbiota». Science (New York, N.Y.) 350 (6264):1079-84.
Wu, Guojun, Ting Xu, Naisi Zhao, Yan Y. Lam, Xiaoying Ding, Dongqin Wei, Jian Fan, et al. 2022. “Two Competing Guilds as a Core Microbiome Signature for Health Recovery.” bioRxiv.
Yonekura, Satoru, Safae Terrisse, Carolina Alves Costa Silva, Antoine Lafarge, Valerio lebba, Gladys Ferrere, Anne-Gaelle Goubet, et al. 2022.«Cancer Induces a Stress Ileopathy Depending on B-Adrenergic Receptors and Promoting Dysbiosis That Contribute to Carcinogenesis». Cancer Discovery, Volume 12, Issue 4, 1128-1151.
Zelante, Teresa, Rossana G. lannitti, Cristina Cunha, Antonella De Luca, Gloria Giovannini, Giuseppe Pieraccini, Riccardo Zecchi, et al. 2013.«Tryptophan Catabolites from Microbiota Engage Aryl Hydrocarbon Receptor and Balance Mucosal Reactivity via Interleukin-22». Immunity 39 (2): 372-85.
Zitvogel, Laurence, Yuting Ma, Didier Raoult, Guido Kroemer, and Thomas F. Gajewski. 2018.«The Microbiome in Cancer Immunotherapy: Diagnostic Tools and Therapeutic Strategies». Science (New York, N. Y.) 359 (6382): 1366-70.

1. A method of treating intestinal dysbiosis in an individual, comprising:
(i) in a sample from said individual comprising intestinal microbiota, assessing the presence or absence of bacterial species of a first species interacting group (“SIG1”) consisting of N₁ bacterial species comprising at least 5, bacterial species selected from the group consisting of Dialister invisus, Enterococcus faecalis, Haemophilus parainfluenzae, Veillonella atypica, Eggerthella lenta, Erysipelatoclostridium ramosum, Enterocloster bolteae, Alloscardovia omnicolens, Bifidobacterium dentium, Campylobacter concisus, Clostridium perfringens, Enterococcus durans, Enterococcus faecium, Klebsiella pneumoniae, Lacticaseibacillus paracasei, Lacticaseibacillus rhamnosus, Lactobacillus delbrueckii, Lactobacillus gasseri, Lactobacillus vaginalis, Lactococcus lactis, Lactococcus laudensis, Ligilactobacillus salivarius, Limosilactobacillus fermentum, Limosilactobacillus oris, Megasphaera micronuciformis, Mogibacterium diversum, Scardovia wiggsiae, Streptococcus anginosus, Streptococcus gordonii, Streptococcus infantis, Streptococcus mutans, Streptococcus parasanguinis, Streptococcus salivarius, Veillonella dispar, Veillonella parvula, Veillonella rogosae, Enterocloster aldensis, Enterocloster asparagiformis, Faecalimonas umbilicata, Gordonibacter urolithinfaciens, Actinomyces graevenitzii, Anaerostipes caccae, Blautia producta, Campylobacter gracilis, Clostridium innocuum, Clostridium scindens, Clostridium symbiosum, Collinsella SGB14754, Enorma massiliensis, Enterocloster clostridioformis, Fournierella massiliensis, Granulicatella adiacens, Hungatella hathewayi, Proteus mirabilis and Streptococcus oralis;
(ii) in a sample from said individual comprising intestinal microbiota, assessing the presence or absence of bacterial species of a second species interacting group (“SIG2”) consisting of N₂ bacterial species comprising at least 5 bacterial species selected from the group consisting of Dorea formicigenerans, Dorea longicatena, Eubacterium ventriosum, Eubacterium rectale, Holdemania filiformis, Parasutterella excrementihominis, Anaerostipes hadrus, Blautia obeum, Blautia wexlerae, Faecalibacterium prausnitzii, Roseburia hominis, Roseburia intestinalis, Roseburia inulinivorans, Ruminococcus bicirculans, Ruminococcus lactaris, Candidatus Cibiobacter qucibialis, Clostridiales bacterium KLE1615, Faecalibacillus intestinalis, Lachnospira eligens, Lacrimispora celerecrescens, Adlercreutzia equolifaciens, Agathobaculum butyriciproducens, Anaerobutyricum hallii, Blautia faecis, Blautia massiliensis, Clostridia unclassified SGB4447, Clostridiaceae bacterium, Clostridium sp AF34 10BH, Clostridium sp AF36 4, Eubacteriaceae bacterium, Fusicatenibacter saccharivorans, Lachnospira pectinoschiza, Lachnospiraceae bacterium, Roseburia faecis, Anaerotignum faecicola, Clostridiaceae bacterium OM08 6BH, Clostridiaceae unclassified SGB4769, Clostridiales unclassified SGB15145, Clostridium fessum, Clostridium sp AM22 11AC, Clostridium sp AM33 3, Clostridium sp AM49 4BH, Coprobacter fastidiosus, Coprococcus comes, Coprococcus eutactus, Eubacterium ramulus, Faecalibacterium SGB15346, Firmicutes bacterium AF16 15, Gemmiger formicilis, Lachnospira sp NSJ 43, Lachnospiraceae bacterium OM04 12BH, Lachnospiraceae bacterium WCA3 601 WT 6H, Lacrimispora amygdalina, Mediterraneibacter butyricigenes, Oscillibacter sp ER4, Phocaeicola massiliensis and Roseburia sp AF02 12;
(iii) calculating a FRNormCount as follows:
$$FRNormCount = \frac{\frac{{N}_{SIG 1}}{N 1}}{\frac{{N}_{SIG 2}}{N 2}} ,$$
wherein N_{SG1} is the number of bacterial species of SIG1 present in the sample and N_{SG2} is the number of bacterial species of SIG2 present in the sample; and/or
(iv) calculating a S score as follows:
$$S = ( \frac{{N}_{SIG 2}}{N 2} - \frac{{N}_{SIG 1}}{N 1} - 1 ) / 2 ,$$
wherein N_{SG1} is the number of bacterial species of SIG1 present in the sample and N_{SG2} is the number of bacterial species of SIG2 present in the sample;
wherein if the FRNormCount is inferior to a predetermined threshold TOPO₁ and/or if the S score is superior to a predetermined threshold S₂, 1 is assigned as a TOPOSCORE and the individual is likely not to have intestinal dysbiosis, and if the FRNormCount is superior to a predetermined threshold TOPO₂ superior to TOPO₁ and/or if the S score is inferior to a predetermined threshold S₁ inferior to S₂, 5 is assigned as a TOPOSCORE and the individual is likely to have intestinal dysbiosis and receives a microbiota-centered intervention (MCI).

2. The method of claim 1, wherein if TOPO₁≤FRNormCount≤TOPO₂ and/or S₁≤S≤S₂ (“grey zone”), the relative abundance of bacteria of the Akkermansia genus (Akk) is measured in a fecal material sample from said individual, wherein:
a) if bacteria of the Akkermansia genus are present in the sample below a predetermined threshold (“Akk superior threshold”), 2 is assigned as a TOPOSCORE and the patient is likely not to have intestinal dysbiosis; and
b) if no Akkermansia is present in the sample, 3 is assigned as a TOPOSCORE and the individual is likely to have intestinal dysbiosis;
c) if bacteria of the Akkermansia genus are present in the sample above the Akk superior threshold, 4 is assigned as a TOPOSCORE and the individual is likely to have intestinal dysbiosis.

3. The method of claim 1, wherein:
(i) the bacterial species of the first species interacting group (“SIG1”) are selected from the group consisting of Veillonella atypica, Erysipelatoclostridium ramosum, Enterocloster bolteae, Enterocloster aldensis, Alloscardovia omnicolens, Bifidobacterium dentium, Campylobacter concisus, Clostridium perfringens, Lacticaseibacillus paracasei, Lactobacillus gasseri, Lactobacillus vaginalis, Ligilactobacillus salivarius, Limosilactobacillus fermentum, Limosilactobacillus oris, Megasphaera micronuciformis, Streptococcus anginosus, Streptococcus gordonii, Streptococcus mutans, Streptococcus parasanguinis, Streptococcus salivarius, Veillonella dispar, Veillonella parvula, Actinomyces graevenitzii, Anaerostipes caccae, Blautia producta, Campylobacter gracilis, Clostridium innocuum, Clostridium scindens, Clostridium symbiosum, Collinsella SGB14754, Enorma massiliensis, Enterocloster clostridioformis, Fournierella massiliensis, Granulicatella adiacens, Hungatella hathewayi, Proteus mirabilis and Streptococcus oralis; and
(ii) the bacterial species of the second species interacting group (“SIG2”) are selected from the group consisting of Dorea formicigenerans, Dorea longicatena, Eubacterium ventriosum, Eubacterium rectale, Anaerostipes hadrus, Blautia wexlerae, Faecalibacterium prausnitzii, Roseburia hominis, Roseburia intestinalis, Roseburia inulinivorans, Ruminococcus bicirculans, Ruminococcus lactaris, Candidatus Cibiobacter qucibialis, Clostridiales bacterium KLE1615, Faecalibacillus intestinalis, Lachnospira eligens, Agathobaculum butyriciproducens, Anaerobutyricum hallii, Blautia massiliensis, Clostridiaceae bacterium, Clostridium sp AF34 10BH, Lachnospira pectinoschiza, Anaerotignum faecicola, Clostridiaceae bacterium OM08 6BH, Clostridiaceae unclassified SGB4769, Clostridiales unclassified SGB15145, Clostridium fessum, Clostridium sp AM22 11AC, Clostridium sp AM33 3, Clostridium sp AM49 4BH, Coprobacter fastidiosus, Coprococcus comes, Coprococcus eutactus, Eubacterium ramulus, Faecalibacterium SGB15346, Firmicutes bacterium AF16 15, Gemmiger formicilis, Lachnospira sp NSJ 43, Lachnospiraceae bacterium OM04 12BH, Lachnospiraceae bacterium WCA3 601 WT 6H, Lacrimispora amygdalina, Mediterraneibacter butyricigenes, Oscillibacter sp ER4, Phocaeicola massiliensis and Roseburia sp AF02 12.

4. The method of claim 1, wherein:
(i) the bacterial species of the first species interacting group (“SIG1”) are selected from the group consisting of Dialister invisus, Enterococcus faecalis, Haemophilus parainfluenzae, Veillonella atypica, Eggerthella lenta, Erysipelatoclostridium ramosum, Enterocloster bolteae, Alloscardovia omnicolens, Bifidobacterium dentium, Campylobacter concisus, Clostridium perfringens, Enterococcus durans, Enterococcus faecium, Klebsiella pneumoniae, Lacticaseibacillus paracasei, Lacticaseibacillus rhamnosus, Lactobacillus delbrueckii, Lactobacillus gasseri, Lactobacillus vaginalis, Lactococcus lactis, Lactococcus laudensis, Ligilactobacillus salivarius, Limosilactobacillus fermentum, Limosilactobacillus oris, Megasphaera micronuciformis, Mogibacterium diversum, Scardovia wiggsiae, Streptococcus anginosus, Streptococcus gordonii, Streptococcus infantis, Streptococcus mutans, Streptococcus parasanguinis, Streptococcus salivarius, Veillonella dispar, Veillonella parvula, Veillonella rogosae, Enterocloster aldensis, Enterocloster asparagiformis, Faecalimonas umbilicata and Gordonibacter urolithinfaciens; and
(ii) the bacterial species of the second species interacting group (“SIG2”) are selected from the group consisting of Dorea formicigenerans, Dorea longicatena, Eubacterium ventriosum, Eubacterium rectale, Holdemania filiformis, Parasutterella excrementihominis, Anaerostipes hadrus, Blautia obeum, Blautia wexlerae, Faecalibacterium prausnitzii, Roseburia hominis, Roseburia intestinalis, Roseburia inulinivorans, Ruminococcus bicirculans, Ruminococcus lactaris, Candidatus Cibiobacter qucibialis, Clostridiales bacterium KLE1615, Faecalibacillus intestinalis, Lachnospira eligens, Lacrimispora celerecrescens, Adlercreutzia equolifaciens, Agathobaculum butyriciproducens, Anaerobutyricum hallii, Blautia faecis, Blautia massiliensis, Clostridia unclassified SGB4447, Clostridiaceae bacterium, Clostridium sp AF34 10BH, Clostridium sp AF36 4, Eubacteriaceae bacterium, Fusicatenibacter saccharivorans, Lachnospira pectinoschiza, Lachnospiraceae bacterium and Roseburia faecis.

5. The method according to claim 1, wherein:
(i) the bacterial species of the first species interacting group (“SIG1”) are selected from the group consisting of Veillonella atypica, Erysipelatoclostridium ramosum, Enterocloster bolteae, Enterocloster aldensis, Alloscardovia omnicolens, Bifidobacterium dentium, Campylobacter concisus, Clostridium perfringens, Lacticaseibacillus paracasei, Lactobacillus gasseri, Lactobacillus vaginalis, Ligilactobacillus salivarius, Limosilactobacillus fermentum, Limosilactobacillus oris, Megasphaera micronuciformis, Streptococcus anginosus, Streptococcus gordonii, Streptococcus mutans, Streptococcus parasanguinis, Streptococcus salivarius, Veillonella dispar and Veillonella parvula; and
(ii) the bacterial species of the second species interacting group (“SIG2”) are selected from the group consisting of Dorea formicigenerans, Dorea longicatena, Eubacterium ventriosum, Eubacterium rectale, Anaerostipes hadrus, Blautia wexlerae, Faecalibacterium prausnitzii, Roseburia hominis, Roseburia intestinalis, Roseburia inulinivorans, Ruminococcus bicirculans, Ruminococcus lactaris, Candidatus Cibiobacter qucibialis, Clostridiales bacterium KLE1615, Faecalibacillus intestinalis, Lachnospira eligens, Agathobaculum butyriciproducens, Anaerobutyricum hallii, Blautia massiliensis, Clostridiaceae bacterium, Clostridium sp AF34 10BH and Lachnospira pectinoschiza.

6. The method of claim 1, wherein:
SIG1 comprises at least Enterocloster bolteae, Erysipelatoclostridium ramosum, Veillonella atypica, Clostridium symbiosum and Hungatella hathewayi, and/or
SIG2 comprises at least 10 bacterial species selected from the group consisting of Anaerostipes hadrus, Blautia wexlerae, Dorea formicigenerans, Dorea longicatena, Eubacterium rectale, Eubacterium ventriosum, Faecalibacterium prausnitzii, Roseburia hominis, Roseburia intestinalis, Roseburia inulinivorans, Ruminococcus bicirculans, Ruminococcus lactaris, Coprococcus comes, Gemmiger formicilis and Phocaeicola massiliensis.

7. The method of claim 1, wherein:
SIG1 comprises at least Dialister invisus, Enterococcus faecalis, Haemophilus parainfluenzae, Veillonella atypica, Eggerthella lenta, Erysipelatoclostridium ramosum and Enterocloster bolteae, and/or
SIG2 comprises at least 10 bacterial species selected from the group consisting of Dorea formicigenerans, Dorea longicatena, Eubacterium ventriosum, Eubacterium eligens, Eubacterium rectale, Holdemania filiformis, Parasutterella excrementihominis, Anaerostipes hadrus, Blautia obeum, Blautia wexlerae, Faecalibacterium prausnitzii, Roseburia hominis, Roseburia intestinalis, Roseburia inulinivorans, Ruminococcus bicirculans and Ruminococcus lactaris.

8. The method of claim 1, further comprising
(v) in a sample from said individual comprising intestinal microbiota, assessing the presence or absence of functional pathways specifically related to SIG1 bacteria in the metagenome, wherein said SIG1-specific pathways are selected from purine nucleobase and pyrimidine deoxynucleotide phosphorylation and degradation, guanosine nucleotide de novo biosynthesis and L histidine degradation,
(vi) in a sample from said individual comprising intestinal microbiota, assessing the presence or absence of functional pathways specifically related to SIG2 bacteria in the metagenome, wherein said SIG2-specific pathways are selected from autophagy-related pathways (polyamines such as S-adenosyl-L-methionine salvage, L-ornithine, L-arginine biosynthesis, putrescine biosynthesis) and sulfur oxidation, superpathway of β-D-glucuronide and D-glucuronate degradation, superpathway of L-alanine and L-aspartate, L-asparagine biosynthesis,
wherein the presence of SIG2-specific functional pathways in the metagenome in the absence of SIG1-specific functional pathways indicates that the individual is of a “SIG2” genotype, and the presence of SIG1-specific functional pathways in the metagenome in the absence of SIG2-specific functional pathways indicates that the person is of a “SIG1” genotype.

9. The method of claim 1, wherein said individual has a cancer amenable to immune-oncology (I-O) therapy.

10. The method of claim 9, wherein said individual has a non small cell lung cancer or a clear cell kidney cancer or an urothelial cancer or a colorectal cancer or a lymphoma.

11. A method of treating a patient having a cancer amenable to immune-oncology (I-O) therapy, comprising assessing, using the method according to claim 2, the patient'≤TOPOSCORE, wherein when the TOPOSCORE≥3, a microbiota-centered intervention (MCI) is administered to the patient before administration of said I-O therapy.

12. The method of claim 11, wherein if the patient has a TOPOSCORE≤2, the I-O therapy is administered to the patient.

13. The method of claim 11, wherein said 1-0 therapy is a treatment with an anti-PD1 antibody, an anti-PD-L1 antibody, an anti-PD-L2 antibody, an anti-CTLA4 antibody and/or a CAR T-cell targeting a tumor antigen, alone or combined with another antineoplastic agent.

14. The method of claim 11, wherein the TOPOSCORE is calculated before beginning the I-O therapy, and optionally after at least partial tumor resection in the individual.

15. (canceled)

16. The method of claim 11, wherein when TOPOSCORE>3, the MCI is to be performed by Fecal Microbial Transplantation (FMT) and if TOPOSCORE=3, the MCI is to be performed by Fecal Microbial Transplantation (FMT) and/or by administering a bacterial composition comprising bacteria of the Akkermansia genus.

17. The method of claim 2, wherein a TOPOSCORE calculated as described in claim 2 is used as a pharmacodynamics tool to monitor the evolution of the intestinal microbiota of an individual receiving an MCI and/or a treatment possibly impacting the intestinal microbiota and/or impacted by the intestinal microbiota.

18. The method of claim 2, wherein a TOPOSCORE calculated as described in claim 2 is used as a theranostic tool for avoiding administering an I-O therapy to a patient likely to have a primary resistance thereto due to intestinal dysbiosis, and/or for stopping such a treatment if the patient develops a secondary resistance thereto, wherein the patient is considered as likely to resist to the treatment when his/her TOPOSCORE is superior or equal to 3.

19. Use of a TOPOSCORE calculated as described in claim 2 for assessing whether a fecal material can be used in an MCI, wherein if the TOPOSCORE>3, the fecal material cannot be used in an MCI and if the TOPOSCORE≤2, the fecal material can be used in an MCI.

20. A kit of parts for performing the method of claim 1, comprising a primer pair and/or a nucleic acid probe specific for each of the bacterial species of the recited SIG1 and SIG2, and a primer pair and/or a nucleic acid probe specific for Akkermansia muciniphila.

21. The kit of parts of claim 20, which comprises a primer pair and/or a nucleic acid probe specific for each one of at least 20 of the following bacterial species, wherein the ratio N_{SIG}1:N_{SIG}2 is about 1:3:
SIG1 bacteria: Streptococcus parasanguinis, Clostridium symbiosum, Streptococcus salivarius, Hungatella hathewayi, Clostridium scindens, Clostridium innocuum, Enterocloster aldensis, Veillonella parvula, Enterocloster bolteae, Erysipelatoclostridium ramosum, Enterocloster clostridioformis, Bifidobacterium dentium, Veillonella dispar and Actinomyces graevenitzii
SIG2 bacteria: Ruminococcus bicirculans, Faecalibacterium prausnitzii, Blautia wexlerae, Roseburia intestinalis, Gemmiger formicilis, Anaerostipes hadrus, Clostridiales bacterium KLE1615, Agathobaculum butyriciproducens, Dorea longicatena, Blautia massiliensis, Eubacterium rectale, Faecalibacterium SGB15346, Clostridium sp AF34 10BH, Lachnospira eligens, Lachnospiraceae bacterium WCA3 601 WT 6H, Clostridium fessum, Anaerobutyricum hallii, Candidatus Cibiobacter qucibialis, Anaerotignum faecicola, Clostridiaceae unclassified SGB4769, Roseburia hominis, Clostridiaceae bacterium, Oscillibacter sp ER4, Clostridiaceae bacterium OM08 6BH, Roseburia inulinivorans, Phocaeicola massiliensis, Lacrimispora amygdalina, Firmicutes bacterium AF16 15, Coprococcus eutactus, Eubacterium ventriosum, Clostridiales unclassified SGB15145, Faecalibacillus intestinalis, Coprococcus comes, Roseburia sp AF02 12, Clostridium sp AM49 4BH, Mediterraneibacter butyricigenes, Dorea formicigenerans, Coprobacter fastidiosus, Ruminococcus lactaris, Lachnospira sp NSJ 43, Clostridium sp AM22 11AC, Lachnospira pectinoschiza, Lachnospiraceae bacterium OM04 12BH, Clostridium sp AM33 3 and Eubacterium ramulus,
as well as a primer pair and/or a nucleic acid probe specific for Akkermansia muciniphila.
